(19) United States
(12) Patent Application Publication
(10) Pub. No.: US 2025/0197827 A1
(43) Pub. Date: Jun. 19, 2025

(54) NOVEL CAS EFFECTOR PROTEINS, GENE EDITING SYSTEMS, AND USES THEREOF

(71) Applicants: GUANGZHOU REFORGENE MEDICINE CO., LTD., Guangzhou (CN); ZHEJIANG SYNSORBIO TECHNOLOGY CO., LTD., Shaoxing (CN)

(72) Inventors: Junbin LIANG, Guangzhou (CN); Liancheng HUANG, Guangzhou (CN); Hui XU, Guangzhou (CN); Yang SUN, Shaoxing (CN); Zhiqin PENG, Guangzhou (CN); Kaiwei SI, Guangzhou (CN); Desheng HUANGFU, Guangzhou (CN)

(73) Assignees: GUANGZHOU REFORGENE MEDICINE CO., LTD., Guangzhou (CN); ZHEJIANG SYNSORBIO TECHNOLOGY CO., LTD., Shaoxing (CN)

(21) Appl. No.: 18/957,929

(22) Filed: Nov. 25, 2024

Related U.S. Application Data
(63) Continuation of application No. PCT/CN2023/096059, filed on May 24, 2023.

(30) Foreign Application Priority Data
May 25, 2022 (CN) 202210576535.8

(51) Int. Cl. C12N 9/22; C12N 15/11; C12N 15/70; C12Q 1/34; C12Q 1/6823

(52) U.S. Cl. CPC C12N 9/22; C12N 15/111; C12N 15/70; C12Q 1/34; C12Q 1/6823; C07K 2319/09; C12N 2310/20; G01N 2333/922

(57) ABSTRACT

Provided are a novel Cas effector protein, a gene editing system, and uses thereof. The Cas effector protein is CasRfg.3 and similar proteins. The CasRfg.3 (also referred to as Ca2) protein of the present disclosure has a relatively shorter amino acid sequence compared to the commonly used SpCas9 protein, allowing it to be easily packaged into small-capacity gene therapy vectors. The CasRfg.3 corresponds to a unique PAM sequence. Additionally, the CasRfg.3 exhibits excellent specificity in targeting and editing nucleic acid sequences, shows good adaptation to temperature variations, and has a wide temperature tolerance range, functioning effectively under high-temperature conditions, such as being capable of cleaving or modifying target nucleic acids at temperatures ranging from 25° C. to 55° C. The novel Cas effector protein of the present disclosure has significant application value in fields like gene therapy.

### CROSS-REFERENCE TO RELATED APPLICATIONS

This application is a Continuation of International Application No. PCT/CN2023/096059, filed on May 24, 2023, which claims priority to Chinese Patent Application No. 202210576535.8, filed on May 25, 2022, the entire contents of each of which are incorporated herein by reference.

### SEQUENCE LISTING

The instant application contains a Sequence Listing which has been submitted electronically in XML format and is hereby incorporated by reference in its entirety. The XML copy, created on Mar. 4, 2025, is named “2025 Mar. 4-Sequence Listing-20954-0001US00”, and is 108,209 bytes in size.

### TECHNICAL FIELD

The present disclosure relates to the technical field of gene editing, and in particular, to novel Cas effector proteins, gene editing systems, and uses thereof.

### BACKGROUND

A CRISPR-CAS system may be categorized into two major classes: Class 1 and Class 2 CRISPR-CAS systems. The Class 1 CRISPR-CAS system utilizes complexes composed of multiple Cas proteins to degrade exogenous nucleic acids, and the Class 2 CRISPR-CAS system employs a single large Cas effector protein to degrade exogenous nucleic acids. The Class 1 CRISPR-CAS system may be further divided into types I, III, and IV, while the Class 2 CRISPR-CAS system may be divided into types II, V, and VI. These six types further segmented into at least 19 subtypes. Most CRISPR-CAS systems possess a Cas1 protein. Many prokaryotes harbor multiple CRISPR-CAS systems, indicating these systems may coexist and potentially share certain components.

Cas9 is one of the first and most representative Cas proteins discovered in Streptococcus pyogenes, and it belongs to the type II of the Class 2 CRISPR-CAS system. SpCas9, derived from Streptococcus pyogenes, is currently the most commonly used Cas9. Cas9 is a DNA endonuclease activated by a small crRNA molecule complementary to a target DNA sequence and a single, separate trans-activating CRISPR RNA (tracrRNA). The crRNA consists of a direct repeat (DR) sequence, responsible for binding the protein with crRNA, and a spacer sequence (guide sequence). The spacer sequence may be artificially designed to complement specific target nucleic acid sequence. In this manner, the CRISPR system can target DNA or RNA targets through the artificial design of the spacer sequence of the crRNA. The crRNA may be fused with the tracrRNA to form a single-guide RNA (sgRNA) for enhanced functionality. The sgRNA, when combined with Cas9, binds to its target DNA and directs Cas9 to bind or cleave the target DNA.

There is an ongoing need to develop reagents with improved sequence-specific nucleic acid detection, cleavage, and modulation that can be applied under various experimental conditions in the fields of genetic research and genome editing.

Current sequence-specific genome editing tools, including Cas9, may not be used under all conditions or in all organisms. For example, sequence-specific nucleases are generally sensitive to heat and are thus unsuitable for use in strictly thermophilic microorganisms. Strictly thermophilic microorganisms refer to those that grow at temperatures between 41° C. and 122° C., and optimally thrive in a range of 45° C.-80° C. Examples of the strictly thermophilic microorganisms include microorganisms used in industrial fermentation or microorganisms for in vitro applications at higher temperatures.

To date, SpCas9 from Streptococcus pyogenes and SaCas9 from Staphylococcus aureus are the most well-characterized and widely used Cas9 for genome engineering. Reports indicate that SpCas9 becomes inactive at temperatures ≥42° C., while SaCas9 unfolds and loses activity at ≥36° C.

SpCas9 is relatively large, at 1,368 amino acids, which significantly limits its application. Researchers from Fudan University truncated SpCas9 to 1,248 amino acids, retaining a certain degree of activity, but its size is still large, making it challenging to package it together with gRNA coding sequences into adeno-associated viruses (AAV).

Moreover, existing Cas proteins have limitations in terms of the gene editing targets they can select, such as being constrained by the insufficient diversity of PAM sequences.

### SUMMARY

The present disclosure aims to provide novel Cas effector proteins, gene editing systems, and uses thereof.

A first aspect of the present disclosure provides a CRISPR-CAS complex comprising:
(1) a Cas9 protein; and
(2) a guide RNA.

The Cas9 protein has a feature selected from any one of (a)-(f) below or any combination thereof:
(a) isolated from a strain of Anaerovibrio sp. of the phylum Firmicutes;
(b) containing or being the polypeptide set forth in SEQ ID NO: 1, or containing or being a conservative variant of the polypeptide set forth in SEQ ID NO: 1 or a homolog thereof;
(c) containing a RuvC domain and an HNH domain;
(d) capable of forming a complex with a guide RNA, binding to a target nucleic acid under the guidance of a guide RNA, or cleaving or modifying the target nucleic acid under the guidance of a guide RNA;
(e) recognizing a PAM sequence as shown in 5′-DDRGDNN-3′, where D represents A, G, or T, R represents A or G, and N represents A, C, G, or T; and
(f) performing any of the functions in (d) at a temperature ranging from 25° C. to 55° C.

In one or more embodiments, the Cas9 protein comprises a polypeptide selected from the group consisting of:
(i) a polypeptide having the amino acid sequence set forth in SEQ ID NO: 1;
(ii) a polypeptide that has at least 90% (preferably at least 92%; more preferably at least 94%; more preferably at least 95%, such as at least 96%, 98%, 99%, or 99.5%) sequence identity to the amino acid sequence of polypeptide set forth in SEQ ID NO: 1;
(iii) a polypeptide formed by substituting, deleting, or adding one or more (e.g., 1-60, preferably 1-50, more preferably 1-40, more preferably 1-30, more preferably 1-20, more preferably 1-15, more preferably 1-10, more preferably 1-5, more preferably 1-3, or 1-2) amino acid residues in the amino acid sequence of the polypeptide in (i), and retaining the function of the polypeptide in (i);
(iv) a fragment of any one of the polypeptides in (i)-(iii) that contains a RuvC domain, a HNH domain, and a domain for binding to a target nucleic acid sequence, and retains the function of the polypeptide in (i); and
(v) a polypeptide formed by adding to the N or C terminus of any of the aforementioned polypeptides (i)-(iv) a sequence including (but not limited to) a tag sequence, a localization sequence (such as a nuclear localization sequence), or a signal peptide sequence.

In one or more embodiments, the polypeptide has a conserved RuvC structural domain sequence.

In one or more embodiments, the polypeptide has a conserved HNH structural domain sequence.

In one or more embodiments, the Cas9 protein is a Cas9-nickase, which is a variant of the Cas9 protein. The Cas9-nickase is capable of binding to a target nucleic acid sequence under the guidance of the guide RNA and cleaving a single strand of the target nucleic acid. Preferably, the Cas9-nickase contains mutations at one or more of D10, E520, H603, N626, H749, or D752 of the amino acid sequence of the polypeptide set forth in SEQ ID NO: 1.

In one or more embodiments, the Cas9 protein is a dCas9 (dead Cas9), which is a variant of the Cas9 protein comprising an inactivated RuvC structural domain and an inactivated HNH structural domain, or a reduced or lost nucleic acid cleavage function thereof. The dCas9 is capable of binding to a target nucleic acid sequence under the guidance of the guide RNA. Preferably, the dCas9 contains mutations at one or more of D10, E520, H603, N626, H749, or D752 of the amino acid sequence of the polypeptide set forth in SEQ ID NO: 1.

In one or more embodiments, the guide RNA includes a guide sequence, a direct repeat sequence (DR sequence), and a trans-activating crRNA (tracrRNA) sequence. The guide sequence is complementary to a target sequence, and the tracrRNA interacts with the Cas9 protein and the DR sequence.

In one or more embodiments, the tracrRNA is linked to the DR sequence to become a scaffold sequence of the guide RNA, and the scaffold sequence has a length in a range of ≤190 nt. The length of the scaffold sequence may also be ≤100 nt, ≤110 nt, ≤120 nt, ≤130 nt, ≤140 nt, ≤150 nt, ≤160 nt, ≤170 nt, or ≤180 nt.

In one or more embodiments, the guide sequence has a length in a range of 10 nt-60 nt. The length of the guide sequence may also be 10 nt-50 nt, 10 nt-40 nt, 10 nt-30 nt, 15 nt-60 nt, 15 nt-50 nt, 15 nt-40 nt, or 15 nt-30 nt.

In one or more embodiments, the guide RNA includes a stem-loop (hairpin) structure. Preferably, the guide RNA includes ≤3 stem-loop (hairpin) structures. More preferably, the guide RNA includes a skeleton containing a stem-loop 1, a stem-loop 2, a stem-loop 3, a bulge 1, and a duplex 1, as shown in FIG. 18.

In one or more embodiments, a stem portion of the stem-loop 1 has 8 to 35 (e.g., 10, 12, 14, 16, 18, 20, 22, 24, 26, 28, 30, 32) base pairs; or a stem portion of the stem-loop 2 has 4 to 12 (e.g., 5, 6, 7, 8, 9, 10, 11) base pairs; or a stem portion of the stem-loop 3 has 3 to 10 (e.g., 4, 5, 6, 7, 8, 9) base pairs; or the duplex 1 has 4 to 11 (e.g., 5, 6, 7, 8, 9, 10) base pairs; or the bulge 1 has 2-8 (e.g., 4, 6) non-complementary bases; or a loop portion of the stem-loop 1 has 4 to 12 (e.g., 5, 6, 7, 8, 9, 10, 11, 12) non-complementary bases; or a loop portion of the stem-loop 2 has 2 to 10 (e.g., 3, 4, 5, 6, 7, 8, 9) non-complementary bases; or a loop portion of the stem-loop 3 has 6 to 20 (e.g., 7, 8, 9, 10, 12, 15, 18, 19) non-complementary bases.

In one or more embodiments, the stem-loop structure includes a skeleton containing the stem-loop 1, the stem-loop 2, the stem-loop 3, the bulge 1, the duplex 1, and optionally other stem-loop structures or segments.

In one or more embodiments, the guide RNA has the nucleotide sequence set forth in SEQ ID NO: 3 or a truncated form thereof. Preferably, the truncated form has a nucleotide sequence set forth in any one of SEQ ID NOs: 4-7.

In one or more embodiments, DNA encoding the guide RNA is included in an expression vector.

In one or more embodiments, the complex (compound) comprises a combination, a composition or a mixture.

In one or more embodiments, the Cas9 protein is capable of performing any one of the following functions at 25° C. to 55° C.: forming a complex with a guide RNA, binding to a target nucleic acid under the guidance of the guide RNA, and cleaving or modifying the target nucleic acid under the guidance of the guide RNA.

In one or more embodiments, the PAM sequence recognized by the Cas9 protein is shown as 5′-DDRGDNN-3′, wherein D represents A, G, or T, R represents A or G, and N represents A, C, G or T.

Another aspect of the present disclosure provides an isolated Cas9 protein. The Cas9 protein has one or more of the features selected from the group consisting of (a) through (f), or any combination thereof:
(a) isolated from a strain of Anaerovibrio sp. of the phylum Firmicutes;
(b) containing or being the polypeptide set forth in SEQ ID NO: 1, or containing or being a conservative variant of the polypeptide set forth in SEQ ID NO: 1 or a homolog thereof;
(c) containing a RuvC domain and an HNH domain;
(d) capable of forming a complex with a guide RNA, binding to a target nucleic acid under the guidance of a guide RNA, or cleaving or modifying the target nucleic acid under the guidance of a guide RNA;
(e) a PAM sequence recognized by the Cas9 protein as shown in 5′-DDRGDNN-3′, where D represents A, G, or T, R represents A or G, and N represents A, C, G, or T; and
(f) performing any of the functions in (d) at a temperature ranging from 25° C. to 55° C.

In one or more embodiments, the Cas9 protein comprises a polypeptide selected from the group consisting of:
(i) a polypeptide having the amino acid sequence set forth in SEQ ID NO: 1;
(ii) a polypeptide that has at least 90% (preferably at least 92%; more preferably at least 94%; more preferably at least 95%, such as at least 96%, 98%, 99%, or 99.5%) sequence identity to the amino acid sequence of polypeptide set forth in SEQ ID NO: 1;
(iii) a polypeptide formed by substituting, deleting, or adding one or more (e.g., 1-60, preferably 1-50, more preferably 1-40, more preferably 1-30, more preferably 1-20, more preferably 1-15, more preferably 1-10, more preferably 1-5, more preferably 1-3, or 1-2) amino acid residues in the amino acid sequence of the polypeptide in (i), and retaining the function of the polypeptide in (i);
(iv) a fragment of any one of the polypeptides in (i)-(iii) that contains a RuvC domain, a HNH domain, and a domain for binding to a target nucleic acid sequence, and retains the function of the polypeptide in (i); and
(v) a polypeptide formed by adding to the N or C terminus of any of the aforementioned polypeptides (i)-(iv) a sequence including (but not limited to) a tag sequence, a localization sequence (such as a nuclear localization sequence), or a signal peptide sequence.

In one or more embodiments, the Cas9 protein is a Cas9-nickase, which is a variant of the Cas9 protein. The Cas9-nickase is capable of binding to a target nucleic acid sequence under the guidance of the guide RNA and cleaves a single strand of the target nucleic acid. Preferably, the Cas9-nickase contains mutations at one or more of D10, E520, H603, N626, H749, or D752 of the amino acid sequence of the polypeptide set forth in SEQ ID NO: 1.

In one or more embodiments, the Cas9 protein is a dCas9 (dead Cas9), which is a variant of the Cas9 protein comprising an inactivated RuvC structural domain and an inactivated HNH structural domain, or a reduced or lost nucleic acid cleavage function thereof. The dCas9 is capable of binding to a target nucleic acid sequence under the guidance of the guide RNA. Preferably, the dCas9 contains mutations at one or more of D10, E520, H603, N626, H749, or D752 of the amino acid sequence of the polypeptide set forth in SEQ ID NO: 1.

In one or more embodiments, the guide RNA includes a guide sequence, a homologous repeat sequence (DR sequence), and a trans-activating crRNA (tracrRNA) sequence. The guide sequence is complementary to a target sequence, and the tracrRNA interacts with the Cas9 protein and the DR sequence.

In one or more embodiments, the tracrRNA is linked to the DR sequence to become a scaffold sequence of the guide RNA, and the scaffold sequence has a length in a range of ≤190 nt. The length of the scaffold sequence may also be ≤100 nt, ≤110 nt, ≤120 nt, ≤130 nt, ≤140 nt, ≤150 nt, ≤160 nt, ≤170 nt, or ≤180 nt.

In one or more embodiments, the guide sequence has a length ranging from 10 nt-60 nt. The length of the guide sequence may also be 10 nt-50 nt, 10 nt-40 nt, 10 nt-30 nt, 15 nt-60 nt, 15 nt-50 nt, 15 nt-40 nt, or 15 nt-30 nt.

In one or more embodiments, the guide RNA includes a stem-loop (hairpin) structure. Preferably, the guide RNA includes ≤3 stem-loop (hairpin) structures. More preferably, the guide RNA includes a skeleton containing a stem-loop 1, a stem-loop 2, a stem-loop 3, a bulge 1, and a duplex 1, as shown in FIG. 18.

In one or more embodiments, the Cas9 protein is capable of performing any one of the following functions at 25° C. to 55° C.: forming a complex with a guide RNA, binding to a target nucleic acid under the guidance of the guide RNA, and cleaving or modifying the target nucleic acid under the guidance of the guide RNA.

In one or more embodiments, the PAM sequence recognized by the Cas9 protein is shown as 5′-DDRGDNN-3′, wherein D represents A, G, or T, R represents A or G, and N represents A, C, G or T.

Another aspect of the present disclosure provides a fusion protein comprising: any one of the Cas9 proteins described above, and a heterologous functional structural domain fused thereto.

In one or more embodiments, the heterologous functional structural domain is fused to an N-terminus, a C-terminus, or an internal portion of the Cas9 protein.

In one or more embodiments, the heterologous functional structural domain is selected from: a reporter protein or detection tag, a localization signal, a deaminase, a DNA-binding domain, an epitope tag, a transcriptional activation domain, a transcriptional repression domain, a nuclease, a methylase, a demethylase, a histone deacetylase (HDAC), a DNA or RNA ligase, or any combination thereof.

In one or more embodiments, the heterologous functional structural domain is selected from: an adenosine deaminase, a cytidine deaminase, a base excision repair inhibitor, a nuclear localization signal (NLS), a nuclear export signal (NES), or any combination thereof.

In one or more embodiments, the heterologous functional structural domain is linked to the Cas9 protein through a linker.

Another aspect of the present disclosure provides a conjugate. The conjugate comprises: any one of the Cas9 proteins described above, and a heterologous functional moiety conjugated thereto.

In one or more embodiments, the heterologous functional moiety is conjugated to a N-terminal, a C-terminal, or an internal portion of the Cas9 protein.

In one or more embodiments, the heterologous functional moiety is selected from: a reporter protein or detection marker, a localization signal, a deaminase, a DNA binding domain, an epitope tag, a transcriptional activation domain, a transcriptional repression domain, a nuclease, a methylase, a demethylase, a histone deacetylase (HDAC), a DNA or RNA ligase, a functional chemical molecule, or any combination thereof.

In one or more embodiments, the heterologous functional moiety is selected from: an adenosine deaminase, a cytidine deaminase (AID), a base excision repair inhibitor, a nuclear localization signal (NLS), a nuclear export signal (NES), or any combination thereof.

In one or more embodiments, there is a linker between the heterologous functional moiety and the Cas9 protein.

Another aspect of the present disclosure provides a guide RNA which guides any of the previously described Cas9 proteins bind to a target sequence. The guide RNA comprises a guide sequence, a direct repeat (DR) sequence, and a trans-activating crRNA (tracrRNA) sequence. The guide sequence is complementary to the target sequence, and the tracrRNA may interact with the Cas9 protein and the DR sequence.

In one or more embodiments, the tracrRNA is ligated to the DR sequence to become a scaffold sequence of the gRNA, and the scaffold sequence has a length in a range of 20 nt-190 nt.

In one or more embodiments, the guide RNA includes a stem-loop (hairpin) structure.

In one or more embodiments, the guide RNA includes ≤3 stem-loop (hairpin) structures.

In one or more embodiments, the stem-loop structure includes a skeleton containing a stem-loop 1, a stem-loop 2, a stem-loop 3, a bulge 1, and a duplex 1, as shown in FIG. 18.

In one or more embodiments, a stem portion of the stem-loop 1 has 8 to 35 (e.g., 10, 12, 14, 16, 18, 20, 22, 24, 26, 28, 30, 32) base pairs; or a stem portion of the stem-loop 2 has 4 to 12 (e.g., 5, 6, 7, 8, 9, 10, 11) base pairs; or a stem portion of the stem-loop 3 has 3 to 10 (e.g., 4, 5, 6, 7, 8, 9) base pairs; or the duplex 1 has 4 to 11 (e.g., 5, 6, 7, 8, 9, 10) base pairs; or the bulge 1 has 2-8 (e.g., 4, 6) non-complementary bases; or a loop portion of the stem-loop 1 has 4 to 12 (e.g., 5, 6, 7, 8, 9, 10, 11, 12) non-complementary bases; or a loop portion of the stem-loop 2 has 2 to 10 (e.g., 3, 4, 5, 6, 7, 8, 9) non-complementary bases; or a loop portion of the stem-loop 3 has 6 to 20 (e.g., 7, 8, 9, 10, 12, 15, 18, 19) non-complementary bases.

In one or more embodiments, the guide RNA has the nucleotide sequence set forth in SEQ ID NO: 3 or a truncated form thereof. Preferably, the truncated form has the nucleotide sequence set forth in any one of SEQ ID NOs: 4-7.

Another aspect of the present disclosure provides an isolated polynucleotide, which encodes any one of the aforementioned Cas9 proteins, any one of the aforementioned fusion proteins, or a protein portion of any one of the aforementioned conjugates.

In one or more embodiments, the polynucleotide is a natural sequence or a codon-optimized sequence.

In one or more embodiments, the polynucleotide of the Cas9 protein has the nucleotide sequence set forth in SEQ ID NO: 2, or a degenerate sequence thereof.

Another aspect of the present disclosure provides a recombinant vector. The recombinant vector contains (a) the polynucleotide as previously described, (b) a polynucleotide encoding any of the previously described guide RNAs, or a combination of (a) and (b).

In one or more embodiments, the polynucleotide is further operably linked to a promoter, an enhancer, and/or a terminator.

In one or more embodiments, the promoter includes a constitutive promoter, an inducible promoter, a broadly expressed promoter, or a tissue-specific promoter.

In one or more embodiments, the vector includes a viral vector or a non-viral vector. Preferably, the vector includes (but is not limited to): a lentiviral vector, an adenoviral vector, an adeno-associated viral vector (AAV), a retroviral vector, a phage vector, or a herpes simplex virus (HSV) vector.

Another aspect of the present disclosure provides a recombinant cell comprising any one of the recombinant vectors previously described.

In one or more embodiments, the recombinant cell includes a eukaryotic cell or a prokaryotic cell.

In one or more embodiments, the eukaryotic cell includes (but is not limited to): a mammalian cell (e.g., a non-human mammalian cell, a human cell), a plant cell, a fungal cell (e.g., yeast), or an insect cell.

In one or more embodiments, the prokaryotic cell includes (but is not limited to): Escherichia coli, Bacillus subtilis, Salmonella, Clostridium, or Streptomyces.

Another aspect of the present disclosure provides a method for preparing any one of the aforementioned Cas9 proteins, any one of the aforementioned fusion proteins, or a protein portion of any one of the aforementioned conjugates. The method comprises: culturing the aforementioned recombinant cell, and isolating the Cas9 protein, the fusion protein, or the protein portion of the conjugate from the culture.

Another aspect of the present disclosure provides any one of the aforementioned Cas9 proteins, any one of the aforementioned fusion proteins, a protein portion of any one of the aforementioned conjugates, or any one of the aforementioned CRISPR-CAS complexes for use in binding, cleaving, or modifying a target nucleic acid sequence, or for use in preparing a reagent for binding, cleaving, or modifying the target nucleic acid sequence.

In one or more embodiments, the Cas9 protein, the fusion protein, or the conjugate forms a complex with the guide RNA to bind, cleave, or modify a nucleic acid sequence at a specific site of the target nucleic acid sequence. Preferably, a PAM sequence, 5′-DDRGDNN-3′, is present near the specific site of the target nucleic acid sequence, where D represents A, G, or T; R represents A or G; and N represents A, C, G, or T.

In one or more embodiments, the Cas9 protein is a Cas9 nuclease, which is used to cleave a target nucleic acid sequence, or is used to prepare a reagent to cleave the target nucleic acid sequence.

In one or more embodiments, the Cas9 protein is a Cas9-nickase, which is used to bind or cleave a target nucleic acid sequence, or is used to prepare a reagent that binds or cleaves the target nucleic acid sequence.

In one or more embodiments, the Cas9 protein is dCas9, which is used to modulate transcriptional activation or transcriptional repression of a target nucleic acid, or to prepare a reagent that modulates transcriptional activation or transcriptional repression of the target nucleic acid.

In one or more embodiments, the target nucleic acid is within a cell. Preferably, the target nucleic acid is: a disease-associated nucleic acid (target), or a plant or animal trait-associated nucleic acid (target).

In one or more embodiments, the target nucleic acid is DNA or RNA.

Another aspect of the present disclosure provides a delivery system. The delivery system comprises: a delivery vector; and any one of the previously described Cas9 proteins, any one of the previously described fusion proteins, any one of the previously described conjugates, any one of the previously described CRISPR-CAS complexes, or any one of the previously described vectors. Preferably, the delivery vector includes (but is not limited to): a nanoparticle, a liposome, and an extracellular vesicle.

Another aspect of the present disclosure provides a method for binding, cleaving, or modifying a target nucleic acid. The method comprises: causing any of the previously described CRISPR-CAS complexes to bind to, cleave, or modify the target nucleic acid.

In one or more embodiments, the target nucleic acid is within a cell. Preferably, the target nucleic acid is a disease-associated nucleic acid (target), or a plant or animal trait-associated nucleic acid (target).

In one or more embodiments, CRISPR-Cas complex (protein+guide RNA) is introduced directly into a cell to bind to the target nucleic acid. Preferably, the Cas9 protein in the CRISPR-Cas complex is processed (including injection) into the cell, or is introduced into the cell via a membrane-translocating peptide, and the guide RNA in the CRISPR-Cas complex is processed (including injection) into the cell.

In one or more embodiments, a polynucleotide encoding the CRISPR-Cas complex or an expression construct (e.g., an expression vector) containing the polynucleotide is introduced into the cell, where the polynucleotide or the expression construct is expressed and forms the CRISPR-Cas complex (protein+guide RNA) and binds to the target nucleic acid.

In one or more embodiments, the target nucleic acid is eukaryotic DNA or eukaryotic RNA.

In one or more embodiments, the eukaryotic DNA or eukaryotic RNA includes (but is not limited to): non-human mammalian DNA or RNA, non-human primate DNA or RNA, human DNA or RNA, plant DNA or RNA, insect DNA or RNA, avian DNA or RNA, reptilian DNA or RNA, rodent DNA or RNA, fish DNA or RNA, worm/nematode DNA or RNA, yeast DNA or RNA, or the like.

In one or more embodiments, the Cas9-nickase or dCas9-targeted target sequence is located in a promoter region.

In one or more embodiments, the method is performed at a temperature ranging from 25° C. to 55° C.

Another aspect of the present disclosure provides a method for detecting the presence of a target nucleic acid in a sample to be tested. The method comprises: causing any one of the previously described CRISPR-CAS complexes to bind to a target nucleic acid. The CRISPR-CAS complex includes any one of the aforementioned fusion proteins or any one of the aforementioned conjugates. The fusion protein or the conjugate contains a Cas9 protein and a detectable label. When the CRISPR-CAS complex binds to the target nucleic acid, the fusion protein or the conjugate containing the Cas9 protein binds to, cleaves, or modifies the target nucleic acid. The presence of the target nucleic acid in the sample to be tested is analyzed by observing the presence of the detectable label. Preferably, the detectable label includes a fluorescent group, a chromogenic agent, a developing agent, or a radioactive isotope.

Another aspect of the present disclosure provides a composition comprising: any one of the aforementioned Cas9 proteins, any one of the aforementioned fusion proteins, any one of the aforementioned conjugates, any one of the aforementioned CRISPR-CAS complexes, any one of the aforementioned vectors, or the aforementioned delivery system.

In one or more embodiments, the composition is a pharmaceutical composition.

In one or more embodiments, preferably, the composition further comprises a physiologically or pharmaceutically acceptable pharmacological carrier.

Another aspect of the present disclosure provides a kit or a drug kit comprising: any one of the aforementioned Cas9 proteins, any one of the aforementioned fusion proteins, any one of the aforementioned conjugates, any one of the aforementioned CRISPR-CAS complexes, any one of the aforementioned vectors, the delivery system, or the composition.

The foregoing is a general description of the present disclosure, and the following individual sections will provide a more detailed description of various aspects of the present disclosure. However, it should be noted that the description of the present disclosure should be understood as follows: to simplify and reduce redundancy, certain embodiments of the present disclosure are described only in one section, or only in the claims or examples. Therefore, it should also be understood that unless otherwise specifically stated or the combination is inappropriate, any embodiment of the present disclosure, including those described only in one aspect, one section, or only in the claims or examples, may be combined with any other embodiment described in the present disclosure.

### BRIEF DESCRIPTION OF THE DRAWINGS

FIG. 1 is a schematic diagram illustrating a domain distribution of a CasRfg.3 protein;

FIG. 2 is a schematic diagram illustrating a PAM sequence recognized by a CasRfg.3 protein;

FIG. 3 is a map of a CasRfg.3-N2-Target plasmid vector;

FIG. 4 is a map of a CasRfg.3-N4-Target plasmid vector;

FIG. 5 is an electrophoresis of CasRfg.3-N2 cleavage products, with fragment lengths of 1589 bp/1605 bp;

FIG. 6 is an electrophoresis of CasRfg.3-N4 cleavage products, with fragment lengths of 1582 bp/1580 bp;

FIG. 7 is a schematic diagram illustrating a sequencing result of CasRfg.3-N2 cleavage fragments;

FIG. 8 is a schematic diagram illustrating a sequencing result of CasRfg.3-N4 cleavage fragments;

FIG. 9 is a schematic diagram illustrating the consistent cleavage performance of CasRfg.3-N2 across multiple repeated experiments;

FIG. 10 is a schematic diagram illustrating the cleavage efficiency of CasRfg.3 at different temperatures;

FIGS. 11A-11G are schematic diagrams illustrating predicted secondary structures of various scaffold sequences by RNAfold;

FIG. 12 is a schematic diagram illustrating results of truncated sgRNAs with different scaffold sequences cleaving a same substrate;

FIG. 13A is a schematic diagram illustrating sgRNA (CasRfg.3-sgRNA-full length) cleavage of Ca2-N2 substrates containing different PAMs;

FIG. 13B is a schematic diagram illustrating sgRNA (tracSL2-1+2-full length) cleavage of Ca2-N2 substrates containing different PAMs;

FIG. 14 is a schematic diagram illustrating the cleavage activity of gRNAs with guide sequences of varying lengths;

FIG. 15 is a schematic diagram comparing multiple sequence alignment of CasRfg.3, SaCas9, and SpCas9 for identification of catalytic residues in CasRfg.3;

FIG. 16 is a map of the pXC09-89-GFPgRNA plasmid used for expressing a Ca9-89 recombinant protein;

FIGS. 17A-17C shows the sequencing results of CBE-Ca2-LacZgRNA02 single-base editing in E. coli β-galactosidase gene, with FIGS. 17B and 17C showing the proportions of A, T, C or G at each base position post-editing; and

FIG. 18 is a schematic diagram illustrating a scaffold structure of a guide RNA comprising a stem-loop 1, a stem-loop 2, a stem-loop 3, a bulge 1, and a duplex 1.

### DETAILED DESCRIPTION

The present inventors, after intensive research and screening, provide a novel Cas effector protein referred to as CasRfg.3 (also referred to as Ca2). The CasRfg.3 protein of the present disclosure has a relatively short amino acid sequence than the currently used SpCas9 protein, making it easily packable into small-capacity gene therapy vectors, such as AAV vectors. Additionally, the CasRfg.3 protein exhibits high specificity in targeting and editing nucleic acid sequences. The CasRfg.3 protein also has good temperature adaptability, with a wide temperature tolerance range, allowing it to function under high-temperature conditions, such as binding, cleaving, or modifying a target nucleic acid at a temperature between 25° C. and 55° C. This novel Cas effector protein has significant application value in fields such as gene therapy.

### Terms

As used herein, “CasRfg.3 effector protein”, “CasRfg.3”, and “Ca2” are used interchangeably. “Cas9 protein” may include “Cas9 nuclease”, or Cas9-nickase (Cas9n) or dCas9 modified on the basis of the Cas9 nuclease.

As used herein, “Cas9 nuclease” generally refers to the Cas9 protein in which the nuclease structural domain is not fully inactivated. Non-limiting examples include wild-type Cas9 and inactivated Cas9 (e.g., a Cas9 mutant that retains more than 99%, 95%, 90%, 80%, 70%, 60%, 50%, 40%, 30%, 20%, 10% or more of the nuclease activity of wild-type Cas9).

As used herein, the term “cleavage/cleaving/cleave” refers to the breaking of a covalent bond (e.g., a covalent phosphodiester bond) in the ribosyl phosphodiester backbone of a polynucleotide, including, but not limited to, the breaking of a single-stranded polynucleotide, the breaking of either single strand of a double-stranded polynucleotide that comprises two complementary single strands, and the breaking of both single strands of a double-stranded polynucleotide that comprises two complementary single strands.

As used herein, the term “modification/modify” is defined to include one or more of the following: nucleic acid base substitution, nucleic acid base deletion, nucleic acid base insertion, nucleic acid methylation, nucleic acid demethylation, and nucleic acid base deamidation.

As used herein, the terms “gRNA”, “guide RNA”, and “sgRNA” are often used interchangeably.

As used herein, the terms “isotropic repeated sequence” and “DR sequence” are used interchangeably.

As used herein, the terms “trans-activated crRNA”, “trans-activated CRISPR RNA” and “tracrRNA” are used interchangeably.

As used herein, the terms “RuvC structural domain”, “RuvC domain”, “RuvC nuclease domain”, and “RuvC nuclease structural domain” are used interchangeably. The terms “HNH structural domain”, “HNH domain”, “HNH nuclease domain”, and “HNH nuclease structural domain” are used interchangeably.

As used herein, the term “target nucleic acid” refers to a polynucleotide having a target sequence. The target nucleic acid may include any polynucleotide, such as DNA (target DNA) or RNA (target RNA). The term “target nucleic acid” refers to a nucleic acid that the gRNA directs the Cas9 protein to, targets, or modifies. The term “target nucleic acid” may be any endogenous or exogenous polynucleotide for a cell (e.g., a eukaryotic cell). For example, the “target nucleic acid” may be a polynucleotide present in eukaryotic cells, a sequence (or a portion thereof) encoding a gene product (e.g., a protein) or a non-coding sequence (or a portion thereof). In some embodiments, the “target nucleic acid” may include one or more disease-associated genes and polynucleotides as well as genes associated with biochemical pathways of signaling. The “disease-associated” gene or polynucleotide refers to any gene or polynucleotide that produces a transcription or translation product at an abnormal level or in an abnormal form in cells of a tissue affected by a disease (e.g., a genetic disease, a chronic disease, an infectious disease, or a cancer, etc.) as compared to control tissues or cells without the disease. In some embodiments, the target nucleic acid is DNA. In some embodiments, the target nucleic acid is an entire chromosomal DNA molecule. The target nucleic acid may be any target nucleic acid (including DNA or RNA) molecule, including naturally occurring nucleic acid molecules with engineered RNA molecules.

As used herein, the term “target sequence” refers to a small sequence in a target nucleic acid molecule that is complementary (fully or partially complementary) or hybridizes to a guide sequence of a gRNA molecule. A length of the target sequence is often tens of bp, for example, about 10 bp, about 20 bp, about 30 bp, about 40 bp, about 50 bp, or about 60 bp.

As used herein, a “specific guide sequence,” also referred to as a “guide sequence,” refers to a segment in the guide RNA that directs a CRISPR-CAS complex to a target nucleic acid sequence for specific binding. The specific guide sequence is complementary (partially complementary or fully complementary) to and/or hybridizes with the target sequence of the target nucleic acid.

Methods for measuring the binding of Cas9 protein, conjugates, or fusion proteins to target nucleic acids are known in the art and include, but are not limited to, chromatin immunoprecipitation assays, electrophoretic mobility shift assays, reporter protein or reporter gene product assays, and microplate capture assays. Similarly, methods for measuring the cleavage or modification of target nucleic acids are known in the art.

As used herein, the term “sequence identity” (also referred to as identity or percent identity) refers to a degree of sequence matching between two polypeptides or two nucleic acids. Comparison is typically made when the two sequences are aligned to generate a highest possible sequence identity. Such alignments may be performed using publicly available and commercially available alignment algorithms and programs, including but not limited to Clustal, MAFFT, Probcons, T-Coffee, Probalign, and BLAST, which may be reasonably selected by those of ordinary skill in the art. A person skilled in the art may able to determine the appropriate parameters for sequence alignment, including any algorithms necessary to achieve optimal alignment or comparison over the full length of the sequences being compared.

As used herein, the term “conserved variant polypeptide” refers to a polypeptide that maintains essentially the same biological function or activity as a wild-type polypeptide (e.g., in the context of the present disclosure, the CasRfg.3 polypeptide isolated from a strain of Anaerovibrio sp. of the phylum Firmicutes). The conserved variant polypeptide may be: (i) a polypeptide in which one or more conserved or non-conserved amino acid residues (preferably conserved amino acid residues) are substituted, where such substituted amino acid residues may or may not be encoded by the genetic code; (ii) a polypeptide with substituent groups at one or more amino acid residues; (iii) a polypeptide formed by fusion of the mature polypeptide with another compound, such as a compound that extends the half-life of the polypeptide (e.g., polyethylene glycol); or (iv) a polypeptide formed by fusion of an additional amino acid sequence to the polypeptide sequence, such as a leader sequence, a secretion sequence, a sequence for purifying the polypeptide, a protein tag sequence, or a fusion protein formed with an antigen IgG fragment. In accordance with the teachings herein, these fragments, derivatives, and analogs fall within the scope known to those skilled in the art. In a more specific manner, the “conservative variant polypeptide” refers to a polypeptide in which up to 100, preferably up to 80, more preferably up to 60, more preferably up to 50, even more preferably up to 30, even more preferably up to 20, even more preferably up to 10, or 5 amino acids are substituted with amino acids of similar or closely related properties compared to the amino acid sequence set forth in SEQ ID NO: 1. The “conservative variant polypeptide” essentially retains the same biological function or activity as the Cas9 protein of the present disclosure.

As used herein, the term “mutation” refers to the substitution of a residue in a sequence (e.g., nucleic acid or amino acid sequence) with another residue, or the replacement of one or more residues in the sequence with a different residue, or the occurrence of a deletion or insertion.

As used herein, the term “insertion/deletion” refers to the insertion or deletion of nucleotide bases in a nucleic acid. Such insertions or deletions may result in a frameshift mutation in a coding region of a gene.

As used herein, the terms “variant” or “mutant” refer to a peptide or polypeptide whose amino acid sequence has been altered by the insertion, deletion, or substitution of one or more amino acids compared to a reference sequence, but which retains at least one biological activity. Variants described in any embodiment of the present disclosure include amino acid sequences that exhibit at least 90%, preferably at least 92%, more preferably at least 94%, even more preferably at least 95%, such as at least 96%, 98%, 99%, or 99.5% sequence identity to the reference sequence (e.g., SEQ ID NO: 1 as described in the present disclosure) while retaining the biological activity of the reference sequence (e.g., as a nuclease). Sequence identity between two aligned sequences may be determined using the BLAST algorithm provided by NCBI, or the like. Mutants also include amino acid sequences having one or more mutations (insertions, deletions, or substitutions) in the amino acid sequence of the reference sequence, while still retaining the biological activity of the reference sequence. The one or more mutations generally refer to 1 to 60 mutations, preferably 1 to 50 mutations, more preferably 1 to 40 mutations, even more preferably 1 to 30 mutations, even more preferably 1 to 20 mutations, even more preferably 1 to 15 mutations, even more preferably 1 to 10 mutations, even more preferably 1 to 5 mutations, or more preferably 1 to 3 mutations, or 1 to 2 mutations. The substitutions are preferably conservative substitutions. For example, in the art, when a residue is substituted by an amino acid of similar or closely related properties, it typically does not alter the function of the protein or polypeptide. “Amino acids of similar or closely related properties” include, for example, families of amino acid residues with similar side chains, such as amino acids with basic side chains (e.g., lysine, arginine, histidine), amino acids with acidic side chains (e.g., aspartic acid, glutamic acid), amino acids with uncharged polar side chains (e.g., glycine, asparagine, glutamine, serine, threonine, tyrosine, cysteine), amino acids with nonpolar side chains (e.g., alanine, valine, leucine, isoleucine, proline, phenylalanine, methionine, tryptophan), amino acids with β-branched side chains (e.g., threonine, valine, isoleucine), and amino acids with aromatic side chains (e.g., tyrosine, phenylalanine, tryptophan, histidine). Therefore, in the polypeptides of the present disclosure, replacing one or more residues with another amino acid residue from the same side-chain class will not substantially affect the activity of the polypeptides.

The “substituted by an amino acid of similar or closely related properties” refers to a substitution between amino acids with similar side chain properties. Thus, the substitution may be, for example, (1) a substitution between aromatic amino acids (Phe, Trp, Tyr), (2) a substitution between nonpolar aliphatic amino acids (Gly, Ala, Val, Leu, Met, Ile, Pro), (3) a substitution between uncharged polar amino acids (Ser, Thr, Cys, Asn, Gln), (4) a substitution between basic amino acids (Lys, Arg, His), or (5) a substitution between acidic amino acids (Asp, Glu).

As used herein, the term “operably linked” refers to a condition in which certain portions of a DNA sequence are capable of regulating or controlling the activity of other portions of the same DNA sequence. For example, if a promoter controls the transcription of a protein-coding sequence, then the promoter is operably linked to the coding sequence.

As used herein, the term “linker” refers to chemical groups or molecules connecting two molecules or portions, such as two domains of a fusion protein, a Cas9 enzyme and a deaminase. In some embodiments, a linker is located between or flanked by two groups, molecules, or other parts and connects them through a covalent bond. In some embodiments, the linker is an amino acid or multiple amino acids (e.g., a peptide or protein). In some embodiments, the linker is an organic molecule, group, polymer, or chemical portion. A length and a type of the linker may be designed as needed.

As used herein, the term “functional chemical molecule” refers to a chemical molecule that is neither an amino acid nor a peptide. Examples of the functional chemical molecule include (but are not limited to) a fluorescent group, a chromogenic agent, a developing agent, or a radioactive isotope.

### Isolated Cas9 Protein

The present disclosure provides an isolated Cas9 protein.

In some embodiments, the Cas9 protein is optionally selected from:
a Cas9 nuclease with nuclease activity;
a Cas9-nickase (Cas9n) or a dCas9 obtained by modifying the Cas9 nuclease; or
a fusion protein or a conjugate comprising the Cas9 nuclease, the Cas9-nickase, or the dCas9.

In some embodiments, the Cas9 protein has one or more of the features selected from the group consisting of (a) through (f), or any combination thereof:
(a) isolated from a strain of Firmicutes species, specifically Anaerovibrio sp.;
(b) containing or being the polypeptide set forth in SEQ ID NO: 1, or containing or being a conservative variant of the polypeptide set forth in SEQ ID NO: 1 or a homolog thereof;
(c) containing a RuvC domain and an HNH domain;
(d) capable of forming a complex with a guide RNA, binding to a target nucleic acid under the guidance of the guide RNA, or cleaving or modifying the target nucleic acid under the guidance of the guide RNA;
(e) a PAM sequence recognized by the Cas9 protein as shown in 5′-DDRGDNN-3′, where D represents A, G, or T, R represents A or G, and N represents A, C, G, or T; and
(f) performing any of the functions in (d) at a temperature ranging from 25° C. to 55° C.

In some embodiments, the Cas9 protein is isolated from a strain of Anaerovibrio sp. of the phylum Firmicutes. In some embodiments, the Cas9 protein is isolated from a species with a genome having an Average Nucleotide Identity (ANI) value ≥95% compared to the genome identified as GCA_902786545.1 in the NCBI database. In some embodiments, the Cas9 protein is isolated from a species with a genome having an ANI value ≥95% compared to the genome of strain RUG13183 in the NCBI database.

The term “Average Nucleotide Identity (ANI)” refers to a measure of a similarity between two genomes at a nucleic acid level, based on the comparison of all orthologous protein-coding genes. For bacteria/archaea, an ANI threshold of 95% is generally used as the criterion to determine whether two genomes belong to a same species (Richter M, Rosselló-Móra R. Shifting the genomic gold standard for the prokaryotic species definition. Proc Natl Acad Sci USA. 2009 Nov. 10; 106 (45): 19126-31). Accordingly, in the context of the present disclosure, species with an ANI value ≥95% to the genomes referenced above are considered to be the same species. Cas9 proteins isolated from such species are homologous, functionally similar, and fall within the scope of the present disclosure.

In some embodiments, the Cas9 protein comprises a polypeptide selected from the group consisting of:
(i) a polypeptide having the amino acid sequence set forth in SEQ ID NO: 1;
(ii) a polypeptide that has at least 90% (preferably at least 92%; more preferably at least 94%; more preferably at least 95%, such as at least 96%, 98%, 99%, or 99.5%) sequence identity to the amino acid sequence of polypeptide set forth in SEQ ID NO: 1;
(iii) a polypeptide formed by substituting, deleting, or adding one or more (e.g., 1-60, preferably 1-50, more preferably 1-40, more preferably 1-30, more preferably 1-20, more preferably 1-15, more preferably 1-10, more preferably 1-5, more preferably 1-3, or 1-2) amino acid residues in the amino acid sequence of the polypeptide in (i), and retaining the function of the polypeptide in (i);
(iv) a fragment of any one of the polypeptides in (i)-(iii) that contains a RuvC domain, a HNH domain, and a domain for binding to a target nucleic acid sequence, and retains the function of the polypeptide in (i); and
(v) a polypeptide formed by adding to the N or C terminus of any of the aforementioned polypeptides (i)-(iv) a sequence including (but not limited to) a tag sequence, a localization sequence (such as a nuclear localization sequence), or a signal peptide sequence.

In some embodiments, the Cas9 protein sequence includes a RuvC nuclease domain and an HNH nuclease domain. The HNH nuclease domain cleaves a complementary strand of a guide sequence, while the RuvC nuclease domain cleaves a non-complementary strand, enabling the specific generation of double-strand breaks (DSB).

In some embodiments, the Cas9 nuclease includes a PI (PAM interaction) domain.

In some embodiments, one or more amino acid residues (e.g., catalytic residues) in the Cas9 protein (including but not limited to the CasRfg.3 protein) may be mutated to modulate its activity in binding, cleaving, or modifying the target nucleic acid. Cas9 generates double-strand breaks via the two nuclease domains, RuvC and HNH. Further, by mutating a key residue, a Cas9-nickase (Cas9n) may be formed, where one of the RuvC or HNH domains is inactivated. The Cas9-nickase, guided by the guide RNA, may bind to the target nucleic acid sequence and cleave the single strand of the target nucleic acid (e.g., target DNA). Thus, to induce a double-strand break at the target nucleic acid, two correctly recognized Cas9n molecules are required to cleave the target nucleic acid, which provides an enhanced specificity compared to the wild-type Cas9 nuclease.

In some embodiments, one or more amino acid residues (e.g., catalytic residues) in the Cas9 protein (including but not limited to the CasRfg.3 protein) may be mutated such that its nuclease activity under the guidance of the guide RNA is completely or partially lost. For example, the RuvC domain and the HNH domain may both be inactivated. This type of modified protein is referred to as dead Cas9 (dCas9). Although the dCas9 has reduced or lost nuclease activity and does not cleave the target nucleic acid, it may still bind to the target nucleic acid and be used to activate or repress transcription of the target nucleic acid. In some embodiments, the dCas9 sequence includes inactivated or reduced activity RuvC and HNH domains. In some embodiments, the nuclease activity of the Cas9 protein may be reduced by modification, such that compared to the wild-type protein, the nuclease activity is at least 50%, 60%, 70%, 80%, 90%, 95%, 97%, or 100% inactivated. The nuclease activity of the Cas9 protein may be reduced by multiple techniques known in the art, such as introducing mutations into the nuclease (catalytic) domains of the protein. In some embodiments, certain catalytic residues responsible for the nuclease activity are identified, and these residues may be substituted with different amino acid residues (e.g., glycine or alanine) to reduce the nuclease activity. In some embodiments, the amino acid substitutions are conservative substitutions (i.e., conservative replacements). In some embodiments, the amino acid substitutions are non-conservative substitutions.

The document titled “Crystal Structure of Staphylococcus aureus Cas9” (http://dx.doi.org/10.1016/j.cell.2015.08.007) is incorporated by reference herein. This publication details the crystal structure of SaCas9 and compares it with the SpCas9 sequence. It also identifies the catalytic residues of SaCas9 and SpCas9 (e.g., Figure S3 of the publication). The catalytic residues of SaCas9 include D10, E477, H557, N580, H701, and D704.

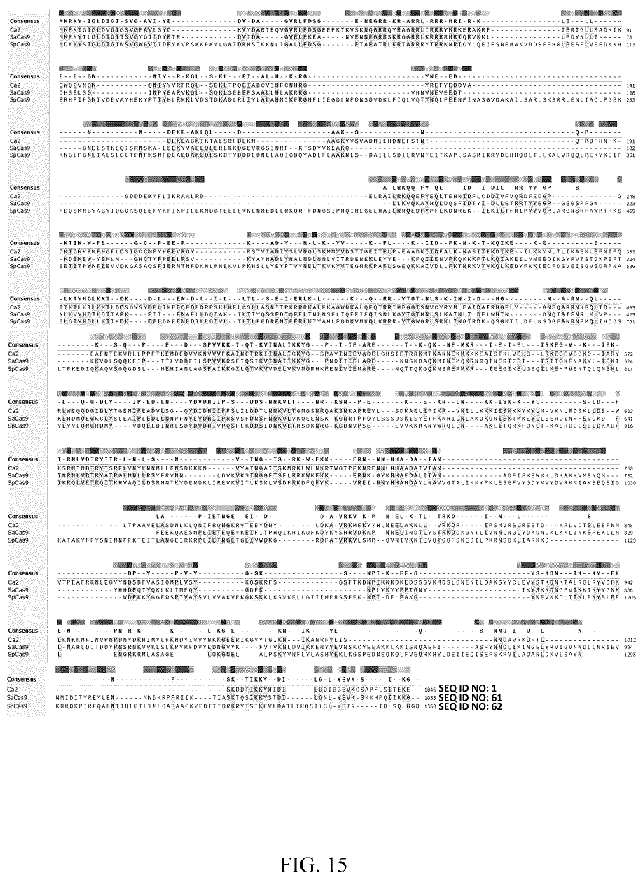

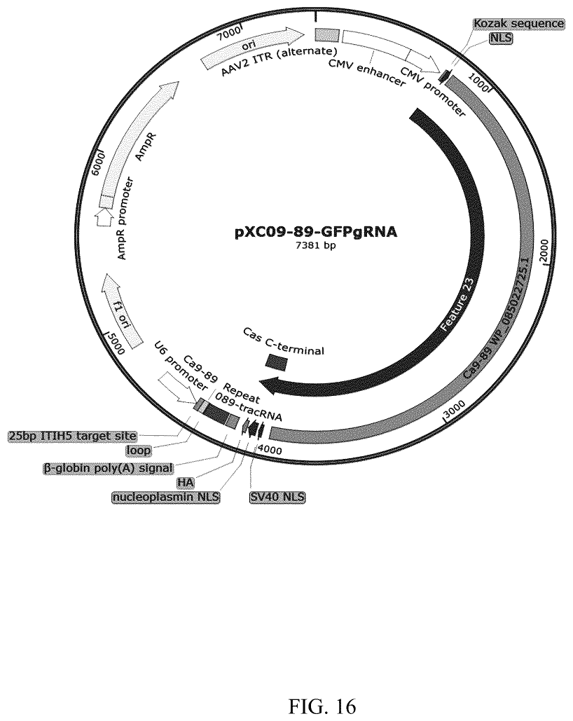

The inventors used the online MAFFT v7.504 program (E-INS-i algorithm, with default parameters for other settings) to perform multiple sequence alignments of the CasRfg.3 (also referred to as Ca2) protein with SaCas9 and SpCas9 (as shown in FIG. 15). At the corresponding positions of the catalytic residues of SaCas9, the catalytic residues of CasRfg.3 were identified as D10, E520, H603, N626, H749, and D752. Therefore, by mutating one or more of these catalytic residues in CasRfg.3, a dead CasRfg.3 or CasRfg.3 nickase may be generated. For other Cas9 proteins of the present disclosure (e.g., conservative variants or homologs of CasRfg.3), this method may also be used to identify corresponding dCas9 or Cas9 nickase.

In some embodiments, the Cas9 protein is fused with a deaminase domain to form a single-base editor capable of editing specific base pairs at target nucleic acid sites. For example, the Cas9 protein is fused with adenosine deaminase to obtain a single-base editor capable of converting an A·T base pair into a G·C base pair. As another example, the Cas9 protein is fused with cytosine deaminase to obtain a single-base editor capable of converting a C·G base pair into a T·A base pair.

In some embodiments, the Cas9-nickase is a variant of the Cas9 nuclease that contains an inactivated RuvC domain or an inactivated HNH domain, and may bind to a target nucleic acid sequence and cleave the single strand of the target nucleic acid (e.g., target DNA) under the guidance of the guide RNA. In some embodiments, the Cas9-nickase contains mutations at one or more positions corresponding to D10, E520, H603, N626, H749, and/or D752 of the amino acid sequence of the polypeptide set forth in SEQ ID NO: 1. In some embodiments, the Cas9-nickase is fused with a deaminase domain to form a single-base editor capable of editing specific base pairs at target nucleic acid sites. For example, the Cas9-nickase is fused with adenosine deaminase to obtain a single-base editor capable of converting an A·T base pair into a G·C base pair. As another example, the Cas9-nickase is fused with cytosine deaminase to obtain a single-base editor capable of converting a C·G base pair into a T·A base pair.

In some embodiments, the dCas9 is a variant of the Cas9 nuclease that contains an inactivated RuvC domain and an inactivated HNH domain, or has reduced or lost nuclease function, and may bind to a target nucleic acid sequence under the guidance of the guide RNA. In some embodiments, the dCas9 contains mutations at one or more positions corresponding to D10, E520, H603, N626, H749, and/or D752 of the amino acid sequence of the polypeptide set forth in SEQ ID NO: 1. In some embodiments, the Cas9-nickase is fused with a deaminase domain to form a single-base editor capable of editing specific base pairs at target nucleic acid sites. For example, the Cas9-nickase is fused with adenosine deaminase to obtain a single-base editor capable of converting an A·T base pair into a G·C base pair. As another example, the Cas9-nickase is fused with cytosine deaminase to obtain a single-base editor capable of converting a C·G base pair into a T·A base pair.

In some embodiments, the present disclosure provides a CasRfg.3 protein, conservative variant polypeptides or homologs thereof, or other proteins/polypeptides/molecules (e.g., detection reagents or pharmaceutical/chemical components) covalently or non-covalently linked to the CasRfg.3 protein, conservative variant polypeptides or homologs thereof. Such additional other proteins/polypeptides/molecules may be conjugated thereto through chemical coupling, gene fusion, or other non-covalent interactions (e.g., biotin-streptavidin binding). These derivative proteins do not affect the function of the original protein, such as its ability to bind to the guide RNA to form a complex and its ability to bind, cleave, or modify the target nucleic acid at a specific site under the guidance of the guide RNA.

In some embodiments, a full-length Cas9 protein, such as a full-length CasRfg.3 protein (SEQ ID NO: 1), conservative variant polypeptides, or homologs thereof, may not be used. Instead, functional fragments of these proteins may be used. The term “functional fragment” refers to a fragment or a derivative of the polypeptide set forth in SEQ ID NO: 1, a conservative variant polypeptide thereof, or a homolog thereof, which has a shorter sequence than the full-length sequence. The missing residues in the functional fragment may be located at the N-terminal, C-terminal, and/or the internal portion. The functional fragment retains at least a portion of the functionality of the polypeptide set forth in SEQ ID NO: 1. Non-limiting examples of the functional fragment include the RuvC domain, the HNH domain, or a PI domain of the CasRfg.3 protein. For example, the RuvC and HNH domains of the CasRfg.3 protein may be used.

In some embodiments, additional domains may be inserted into the Cas9 protein while maintaining some of the original Cas9 protein's functions, such as its ability to form a complex with the guide RNA, target and bind to the target nucleic acid, cleave the target nucleic acid, or modify the target nucleic acid. For example, a deaminase domain, a transcriptional activation domain (e.g., VP64 or VPR), a transcriptional repression domain (e.g., KRAB or SID domain), or a nuclease structural domain (e.g., FokI) may be inserted into the Cas9 protein. These modified proteins are also within the scope of the present disclosure.

In some embodiments, the CasRfg.3 protein, conservative variant polypeptides, or homologs thereof may not be naturally occurring and may differ from naturally occurring sequences by at least one amino acid.

In some embodiments, the Cas9 protein may form a complex with the guide RNA provided in the present disclosure.

In some embodiments, the Cas9 protein may bind to the target nucleic acid under the guidance of the guide RNA provided in the present disclosure.

In some embodiments, the Cas9 protein may cleave or modify the target nucleic acid under the guidance of the guide RNA provided in the present disclosure.

In some embodiments, the Cas9 protein may be used to modulate transcriptional activation or transcriptional repression of the target nucleic acid under the guidance of the guide RNA provided in the present disclosure.

In some embodiments, the Cas9 protein may be used in combination with the guide RNAs in the present disclosure to regulate transcriptional activation or transcriptional repression of the target nucleic acid.

In some embodiments, in a temperature range of 25° C. to 55° C., the Cas9 protein may:
form a complex with the guide RNA of the present disclosure;
bind to the target nucleic acid under the guidance of the guide RNA of the present disclosure;
cleave or modify the target nucleic acid under the guidance of the guide RNA of the present disclosure; or
combine with the guide RNA of the present disclosure to regulate the transcriptional activation or repression of the target nucleic acid.

Further, the temperature range may be 35° C. to 55° C. Still further, the temperature range may be 37° C. to 55° C., 40° C. to 55° C., 45° C. to 55° C., or 50° C. to 55° C.

In some embodiments, a PAM sequence recognized by the Cas9 protein (including, but not limited to, CasRfg.3) is 5′-DDRGDNN-3′ (D is A, G, or T; R is A or G; N is A, C, G, or T). Further, in some embodiments, a PAM sequence corresponding to the Cas9 protein (including but not limited to CasRfg.3) is selected from: 5′-ATAGGCT-3′, 5′-AAGGCT-3′, 5 ‘-AGGGTCG-3’, 5′-GAGGTCC-3′, 5′-TTGGGCT-3′, 5′-TGGGGTA-3′, 5′-TGGGGGC-3′, 5′-GGGGGGA-3′, 5′-AGAGGGAT-3′, 5′-AGGGTCC-3′, 5′-ATAGGGA-3′, 5′-GTAGAAT-3′, 5 ‘-GGGGAAG-3’, 5′-TAAGGTT-3′, 5′-TGGGTAG-3′, or 5′-GGGGGAT-3′.

In some embodiments, the Cas9 protein (including but not limited to the CasRfg.3 protein) does not possess substantial or detectable additional nuclease activity (e.g., DNase/RNase activity). Here, “additional nuclease activity” refers to non-specific nuclease activity. For example, a complex containing the Cas9 protein, when activated by binding to the target nucleic acid (e.g., target DNA), undergoes a conformational change, causing the complex to act as a non-specific nuclease, cleaving and/or degrading nearby nucleic acid molecules (e.g., DNA or RNA molecules), thus exerting an “additional” effect.

### Conjugate

The present disclosure provides a conjugate, comprising: the Cas9 protein of the present disclosure, and a heterologous functional moiety conjugated thereto.

In some embodiments, the Cas9 protein is a Cas9 nuclease with nuclease activity (including but not limited to the unmutated wild-type Cas9 protein, e.g., the CasRfg.3 protein), the Cas9-nickase (Cas9n, nCas9), the dCas9, or derivatives or functional fragments thereof, and the Cas9 protein may be conjugated with one or more heterologous functional moieties. These heterologous functional moieties may have various activities, such as a methyltransferase activity, a demethylase activity, a deaminase activity, a transcriptional activation activity, a transcriptional repression activity, a transcription release factor activity, a histone modification activity, a RNA cleavage activity, a DNA cleavage activity, a nucleic acid binding activity, a base editing activity, and a switchable activity (e.g., photo-inducible).

The heterologous functional moieties may include, but are not limited to: localization signals (e.g., a nuclear localization signal (NLS), a nuclear export signal (NES)), reporter proteins or reporter gene products (e.g., GST, HRP, CAT, GFP, HcRed, DsRed, CFP, YFP, BFP), tags or detection labels (e.g., fluorescent dyes such as FITC or DAPI), targeting moieties, DNA-binding domains (e.g., MBP, Lex A DBD, Gal4 DBD), antigenic epitope tags (e.g., His, myc, V5, FLAG, HA, VSV-G, Trx, etc.), transcriptional activation domains (e.g., VP64 or VPR), transcriptional repression domains (e.g., KRAB or SID portions), nucleases (e.g., FokI), deaminase or deaminase domains (e.g., ADAR1, ADAR2, APOBEC, AID, or TAD), base excision repair inhibitors (e.g., uracil-DNA glycosylase inhibitor (UGI)), methyltransferases, demethylases, transcription release factors, HDAC, ssRNA cleavage domains, dsRNA cleavage domains, ssDNA cleavage domains, dsDNA cleavage domains, DNA or RNA ligases, functional chemical molecules, or any combination thereof.

For example, the conjugate may include one or more NLSs, which may be located at the N-terminus, the C-terminus, and/or within the Cas9 protein.

In some embodiments, the conjugate may be obtained through a covalent bond or a non-covalent bond.

In some embodiments, the conjugate may be obtained through a linker, i.e., the heterologous functional moiety is linked to the Cas9 protein through a linker. The linker, the heterologous functional moiety, and the Cas9 protein may be connected via covalent or non-covalent bonds. Non-limiting examples of the linker include amino acids, peptides, amino acid derivatives (e.g., Ahx, β-Ala, GABA, or Ava), PEG, or the like.

Generally, the conjugation of the heterologous functional moiety does not affect the functionality of the original protein, such as its ability to bind to the guide RNA to form a complex or its ability to bind to or cleave target nucleic acids at specific sites under the guidance of the guide RNA.

In some embodiments, the conjugate may form a complex with the guide RNA of the present disclosure.

In some embodiments, the conjugate may bind to the target nucleic acid under the guidance of the guide RNA. For example, when conjugated with a nuclear localization signal (NLS), the conjugate may enter the nucleus of a eukaryotic cell under the guidance of the NLS and bind to the target nucleic acid.

In some embodiments, the conjugate may cleave or modify the target nucleic acid under the guidance of the guide RNA. For example, the Cas9 protein of the present disclosure may be conjugated with an NLS, and under the guidance of the NLS, enter the nucleus of a eukaryotic cells to cleave the target nucleic acid. Subsequently, the target nucleic acid may be further modified via a NHEJ or HDR repair mechanism (e.g., resulting in base insertion/deletion (indel) in the target nucleic acid) of the cell. For example, the Cas9 protein (e.g., the Cas9-nickase) of the present disclosure may be conjugated with a deaminase or with both a deaminase and a uracil-DNA glycosylase inhibitor (UGI), thus creating a single-base editor containing the Cas9 protein of the present disclosure. The single-base editor may also be conjugated with an NLS. The single-base editor may modify the target nucleic acid (e.g., perform base substitutions) under the guidance of the guide RNA of the present disclosure.

In some embodiments, the conjugate may be combined with the guide RNA to regulate transcriptional activation or repression of the target nucleic acid. For example, the Cas9 protein (e.g., the dCas9) of the present disclosure may be conjugated with a transcriptional activation domain (e.g., VP64 or VPR) or a transcriptional repression domain (e.g., KRAB or SID portion), and then combined with the guide RNA (e.g., targeting the promoter or enhancer regions of a gene of interest) to regulate transcriptional activation or repression of the target nucleic acid.

In some embodiments, the conjugate may, within a temperature range of 25° C. to 55° C.: form a complex with the guide RNA of the present disclosure;
bind to the target nucleic acid under the guidance of the guide RNA of the present disclosure;
cleave or modify the target nucleic acid under the guidance of the guide RNA of the present disclosure; or
be combined with the guide RNA of the present disclosure to regulate transcriptional activation or repression of the target nucleic acid.

Further, the temperature range may be 35° C. to 55° C. Furthermore, the temperature range is 37° C.˜55° C., 40° C.˜55° C., 45° C.˜55° C., or 50° C.˜55° C.

In some embodiments, a PAM sequence recognized by the conjugate is 5′-DDRGDNN-3′ (D is A, G, or T; R is A or G; N is A, C, G, or T). Further, in some embodiments, a PAM sequence corresponding to the conjugate is selected from: 5′-ATAGGCT-3′, 5′-AAGGCT-3′, 5′-AGGGTCG-3′, 5′-GAGGTCC-3′, 5′-TTGGGCT-3′, 5′-TGGGGTA-3′, 5′-TGGGGGC-3′, 5′-GGGGGGA-3′, 5′-AGAGGAT-3′, 5′-AGGGTCC-3′, 5′-ATAGGGA-3′, 5′-GTAGAAT-3′, 5′-GGGGAAG-3′, 5′-TAAGGTT-3′, 5′-TGGGTAG-3′, and 5′-GGGGGAT-3′.

### Fusion Protein

The present disclosure provides a fusion protein, comprising: the Cas9 protein described in any of the previous embodiments, and a heterologous functional domain fused thereto.

In some embodiments, the Cas9 protein is a Cas9 nuclease with nuclease activity (including but not limited to a wild-type Cas9 protein that has not undergone mutations [e.g., the CasRfg.3 protein], as well as active mutants of the wild-type Cas9 protein), Cas9-nickase (Cas9n, nCas9), dCas9, or derivatives or functional fragments thereof. The Cas9 protein may be fused or associated with one or more heterologous functional domains (e.g., through fusion proteins, linker peptides, etc.). These functional domains may exhibit various activities, such as a methyltransferase activity, a demethylase activity, a deaminase activity, a transcriptional activation activity, a transcriptional repression activity, a transcription release factor activity, a histone modification activity, a RNA cleavage activity, a DNA cleavage activity, a nucleic acid binding activity, a base editing activity, and a switchable activity (e.g., photo-inducible).

The heterologous functional domains may include, but are not limited to: localization signals (e.g., nuclear localization signal NLS, nuclear export signal NES), reporter proteins or reporter gene products (e.g., GST, HRP, CAT, GFP, HcRed, DsRed, CFP, YFP, BFP), labels or detection markers (e.g., FITC or DAPI fluorescent dyes), targeting regions, DNA-binding domains (e.g., MBP, Lex A DBD, Gal4 DBD), antigen epitope tags (e.g., His, myc, V5, FLAG, HA, VSV-G, Trx, etc.), transcriptional activation domains (e.g., VP64 or VPR), transcriptional repression domains (e.g., KRAB or SID regions), nucleases (e.g., FokI), deaminase or deamination domains (e.g., ADAR1, ADAR2, APOBEC, AID, or TAD), base excision repair inhibitors (e.g., uracil-DNA glycosylase inhibitor (UGI)), methylases, demethylases, transcriptional release factors, HDACs, ssRNA cleavage activity domains, dsRNA cleavage activity domains, ssDNA cleavage activity domains, dsDNA cleavage activity domains, DNA or RNA ligases, or any combination of the above.

In some embodiments, the Cas9 protein is a Cas9 nuclease with nuclease activity (including but not limited to the wild-type Cas9 protein [e.g., CasRfg.3 protein], as well as active mutants of the wild-type Cas9 protein). The Cas9 nuclease is fused with a deaminase domain to form a single-base editor, which may edit bases at specific sites in a target nucleic acid. For example, the Cas9 nuclease is fused with adenosine deaminase to obtain a single-base editor capable of converting an A·T base pair into a G·C base pair. As another example, the Cas9 nuclease is fused with cytosine deaminase to obtain a single-base editor capable of converting A C·G base pair into a T·A base pair.

In some embodiments, the Cas9 protein is a Cas9-nickase, which includes an inactive RuvC domain or an inactive HNH domain. Under the guidance of a guide RNA, the Cas9-nickase may bind to and cleave the single strand of a target nucleic acid sequence (e.g., target DNA). In some embodiments, the Cas9-nickase undergoes mutations at the D10, E520, H603, N626, H749, or D752 positions of the polypeptide set forth in SEQ ID NO: 1. In some embodiments, the Cas9-nickase is fused with a deaminase domain to form a base editor, which may edit bases at specific sites of the target nucleic acid. For example, the Cas9-nickase is fused with adenosine deaminase to obtain a single-base editor capable of converting an A·T base pair into a G·C base pair. As another example, the Cas9-nickase is fused with cytosine deaminase to obtain a single-base editor capable of converting A C·G base pair into a T·A base pair.

In some embodiments, the Cas9 protein is a dCas9, which includes an inactive RuvC domain and an inactive HNH domain, or has a reduced or lost nuclease activity. Under the guidance of a guide RNA, the dCas9 may bind to the target nucleic acid sequence. In some embodiments, the dCas9 undergoes mutations at the D10, E520, H603, N626, H749, or D752 positions of the polypeptide set forth in SEQ ID NO: 1. In some embodiments, the Cas9-nickase is fused with a deaminase domain to form a base editor, which may edit bases at specific sites of the target nucleic acid. For example, the dCas9 is fused with adenosine deaminase to obtain a single-base editor capable of converting an A·T base pair into a G. C base pair. As another example, the dCas9 is fused with cytosine deaminase to obtain a single-base editor capable of converting A C·G base pair into a T·A base pair.

In some embodiments, the fusion protein may include multiple (e.g., 2, 3, 4, 5, 6, 7, 8, or more) identical or different functional domains. For example, the fusion protein may include one or more nuclear localization signals (NLS), such as 1, 2, 3, 4, 5, or more NLS, for example ≥1, ≥2, ≥3, or ≥4 NLS. These NLS may be located at the N-terminus, C-terminus, or internally within the Cas9 protein.

In some embodiments, the heterologous functional domain is connected to the Cas9 protein via a linker. The linker, the heterologous functional domain, and the Cas9 protein may be connected via covalent or non-covalent bonds. Non-limiting examples of linkers include amino acids, peptides, amino acid derivatives (e.g., Ahx, β-Ala, GABA, or Ava), etc. In some embodiments, the functional domain is associated or fused with a linker sequence (e.g., a flexible linker sequence or a rigid linker sequence).

In some embodiments, the fusion of the heterologous functional domain does not affect the original function of the Cas9 protein, such as its ability to form a complex with the guide RNA described in the present disclosure, or its ability to bind or cleave the target nucleic acid at specific sites under the guidance of the guide RNA.

The position of one or more functional domains on the Cas9 protein enables the functional domains to be correctly spatially oriented, thereby imparting desired functional effects on the target. For example, if the functional domain is a transcriptional activator (e.g., VP16, VP64, or p65), the transcriptional activator is positioned to spatially orient in a way that influences the transcription of a target. Similarly, a transcriptional repressor may be positioned to influence the transcription of the target, and a nuclease (e.g., FokI) may be positioned to cleave or partially cleave the target. In some embodiments, the functional domain is located at the N-terminus of the Cas9 protein. In some embodiments, the functional domain is located at the C-terminus of the Cas9 protein. In some embodiments, the functional domain is located at both the N-terminus and C-terminus of the Cas9 protein. In some embodiments, the dCas9/nCas9 is modified, and the modified dCas9/nCas9 contains a first functional domain at the N-terminus and a second functional domain at the C-terminus.

In some embodiments, the fusion protein may form a complex with the guide RNA described in the present disclosure.

In some embodiments, the fusion protein may bind to the target nucleic acid under the guidance of the guide RNA described in the present disclosure. For example, the fusion protein may be fused with an NLS, enter the nucleus of a eukaryotic cell under the guidance of the NLS, and bind to the target nucleic acid.

In some embodiments, the fusion protein may cleave or modify the target nucleic acid under the guidance of the guide RNA described in the present disclosure. For example, the Cas9 protein of the present disclosure may be fused with an NLS, enter the nucleus of a eukaryotic cell under the guidance of the NLS, and cleave the target nucleic acid. Subsequently, the target nucleic acid may be further modified through an endogenous NHEJ or HDR repair mechanism (e.g., resulting in base insertion or deletion [indel] in the target nucleic acid) of the cell. For example, the Cas9 protein (e.g., the Cas9-nickase) of the present disclosure may be fused with a deaminase, or with both a deaminase and a uracil-DNA glycosylase inhibitor (UGI), forming a base editor containing the Cas9 protein of the present disclosure. The base editor may also be fused with an NLS. The base editor may modify (e.g., performing base substitution) the target nucleic acid under the guidance of the guide RNA of the present disclosure.

In some embodiments, the fusion protein may be combined with the guide RNA to regulate transcriptional activation or transcriptional repression of the target nucleic acid. For example, the Cas9 protein (e.g., the dCas9) of the present disclosure may be fused with a transcriptional activation domain (e.g., VP64 or VPR) or a transcriptional repression domain (e.g., KRAB or SID), and then combined with the guide RNA (e.g., targeting the promoter region or enhancer region of a gene of interest) to regulate transcriptional activation or transcriptional repression of the target nucleic acid.

In some embodiments, the fusion protein, within a temperature range of 25° C. to 55° C., may:
form a complex with the guide RNA described in the present disclosure;
bind to a target nucleic acid under the guidance of the guide RNA described in the present disclosure;
cleave or modify the target nucleic acid under the guidance of the guide RNA described in the present disclosure; or
be combined with the guide RNA described in the present disclosure to regulate transcriptional activation or transcriptional repression of the target nucleic acid.

Further, the temperature range may be 35° C.-55° C. Further, the temperature may be 37° C.-55° C., 40° C.-55° C., 45° C.-55° C., or 50° C.-55° C.

In some embodiments, a PAM sequence recognized by the fusion protein is 5′-DDRGDNN-3′ (D is A, G, or T; R is A or G; N is A, C, G, or T). Further, in some embodiments, a PAM sequence corresponding to the fusion protein is selected from: 5′-ATAGGCT-3′, 5′-AAGGCT-3′, 5′-AGGGTCG-3′, 5′-GAGGTCC-3′, 5′-TTGGGCT-3′, 5′-TGGGGTA-3′, 5′-TGGGGGC-3′, 5′-GGGGGGA-3′, 5′-AGAGGAT-3′, 5′-AGGGTCC-3′, 5′-ATAGGGA-3′, 5′-GTAGAAT-3′, 5′-GGGGAAG-3′, 5′-TAAGGTT-3′, 5′-TGGGTAG-3′, 5′-GGGGGAT-3′.

### CRISPR-CAS Complex

The present disclosure also provides a CRISPR-CAS complex comprising:
(1) any Cas9 protein described in the present disclosure; and
(2) a guide RNA.

In some embodiments, the Cas9 protein is a Cas9 nuclease with nuclease activity, or a Cas9-nickase (Cas9n) or dCas9 that is a modified form of the Cas9 nuclease, or a fusion protein or conjugate containing the Cas9 nuclease, Cas9-nickase, or dCas9.

In some embodiments, the Cas9 protein has a feature selected from any one of (a)-(f) below or any combination thereof:
(a) isolated from a strain of Anaerovibrio sp. of the phylum Firmicutes;
(b) containing or being the polypeptide set forth in SEQ ID NO: 1, or containing or being a conservative variant of the polypeptide set forth in SEQ ID NO: 1 or a homolog thereof;
(c) containing a RuvC domain and an HNH domain;
(d) capable of forming a complex with a guide RNA, binding to a target nucleic acid under the guidance of the guide RNA, or cleaving or modifying the target nucleic acid under the guidance of the guide RNA;
(e) a PAM sequence recognized by the Cas9 protein as shown in 5′-DDRGDNN-3′, where D represents A, G, or T, R represents A or G, and N represents A, C, G, or T; and
(f) performing any of the functions in (d) at a temperature ranging from 25° C. to 55° C.

The CRISPR-Cas complex of the present disclosure may be used in various applications, such as gene editing-based therapies. Since the CasRfg.3 protein is significantly smaller than many Cas9 proteins in the field, nucleic acids encoding the CasRfg.3 protein, its conservative variant polypeptides, or homologs thereof, as well as the corresponding guide RNA encoding sequences, are more suitable for packaging into delivery systems (e.g., a AAV vector) with size constraints.

### Guide RNA

The present disclosure also provides a guide RNA, capable of interacting with or forming a complex with the Cas9 protein of the present disclosure, or directing the binding of the Cas9 protein of the present disclosure to a target nucleic acid, or guiding the Cas9 protein to bind to the target nucleic acid, or guiding the Cas9 protein to cleave or modify the target nucleic acid, or combining with the Cas9 protein to regulate transcriptional activation or transcriptional repression of the target nucleic acid.

In some embodiments, the guide RNA includes a guide sequence and a direct repeat (DR) sequence.

In some embodiments, the guide RNA comprises a guide sequence, a direct repeat (DR) sequence, and a trans-activating crRNA (tracrRNA) sequence.

The guide sequence interacts with, binds to, complements (partially or fully complements), or hybridizes with a target sequence on the target nucleic acid; or the guide sequence directs the Cas9-guide RNA complex to the target nucleic acid sequence for specific binding. The tracrRNA may interact or bind with the Cas9 protein, and the tracrRNA may complement (partially or fully complement), interact, or bind with the DR sequence.

In some embodiments, the CRISPR system described in the present disclosure includes the guide RNA. The CRISPR system may include one or more guide RNAs (e.g., 1 to 12; specifically, 1, 2, 3, 4, 5, 6, 7, 8, or more RNA guides).

In some embodiments, the DR sequence is linked to the 3′ end of the guide sequence, e.g., by a covalent bond.

The DR sequence and the guide sequence are collectively referred to as crRNA. The tracrRNA and the crRNA may be covalently linked. In some embodiments, the tracrRNA is connected to the DR sequence to form a scaffold sequence of the gRNA, for example, by embedding one, two, three, four, five, six, or more nucleotides to connect the tracrRNA with the DR sequence, thereby forming a single-molecule gRNA. The sequence from the 5′ end to the 3′ end is arranged as follows: guide sequence→DR sequence→tracrRNA sequence.

A length of the scaffold sequence ranges from 20 nt to 190 nt.

Preferably, in some embodiments, the crRNA, the tracrRNA, or the scaffold sequence may form one or more stem-loop structures.

In some embodiments, the scaffold sequence may form one, two, three, four, five, six, or more stem-loop structures. In some embodiments, the scaffold sequence may form one, two, or three stem-loop structures. In some embodiments, the scaffold sequence may form ≥1, ≥2, or ≥3 stem-loop structures. In some embodiments, the scaffold sequence may form ≤1, ≤2, or ≤3 stem-loop structures.

In some embodiments, the scaffold sequence may form one or more stem-loop structures, a core structure of the stem-loop structure is shown in FIG. 18.

In some embodiments, the 5′ end of the scaffold sequence is involved in forming a duplex structure.

In some embodiments, the scaffold sequence, from the 5′ end to the 3′ end, sequentially contains a duplex, a bulge, and a stem-loop 1. In some embodiments, the scaffold sequence, from the 5′ end to the 3′ end, sequentially contains a duplex, a bulge, a stem-loop 1, an optional linker sequence, and a stem-loop 2. In some embodiments, the scaffold sequence, from the 5′ end to the 3′ end, sequentially contains a duplex, a bulge, a stem-loop 1, an optional linker sequence 1, a stem-loop 2, an optional linker sequence 2, and a stem-loop 3.

In some embodiments, the length of the scaffold sequence is ≥20 nt, ≥30 nt, ≥40 nt, ≥50 nt, ≥60 nt, ≥70 nt, ≥80 nt, ≥90 nt, ≥100 nt, ≥110 nt, ≥120 nt, ≥130 nt, ≥140 nt, ≥150 nt, or ≥160 nt. In some embodiments, the length of the scaffold sequence is ≤100 nt, ≤110 nt, ≤120 nt, ≤130 nt, ≤140 nt, ≤150 nt, ≤160 nt, ≤170 nt, ≤180 nt, or ≤190 nt. The term “nt” refers to nucleotides.

In some embodiments, the length of the scaffold sequence is in a range of 20 nt-190 nt, 20 nt-160 nt, 20 nt-140 nt, 20 nt-120 nt, 20 nt-110 nt, 20 nt-100 nt, 40 nt-160 nt, 40 nt-140 nt, 40 nt-120 nt, 40 nt-110 nt, 40 nt-100 nt, 60 nt-160 nt, 60 nt-140 nt, 60 nt-120 nt, 60 nt-110 nt, 60 nt-100 nt, 80 nt-160 nt, 80 nt-140 nt, 80 nt-120 nt, 80 nt-110 nt, 80 nt-100 nt, 100 nt-160 nt, 100 nt-140 nt, or 100 nt-120 nt.

In some embodiments, a length of the guide sequence is in a range of 10 nt-60 nt, 10 nt-50 nt, 10 nt-40 nt, 10 nt-30 nt, 15 nt-60 nt, 15 nt-50 nt, 15 nt-40 nt, or 15 nt-30 nt.

In some implementations, the length of the guide sequence ranges from 16 nt-30 nt. In some implementations, the length of the guide sequence ranges from 18 nt-25 nt. In some implementations, the length of the guide sequence ranges from 20 nt-24 nt. In some embodiments, the length of the guide sequence is 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt.

In some embodiments, the guide RNA comprises the nucleotide sequence set forth in SEQ ID NO: 3 or a truncated version thereof. In some embodiments, the truncated version includes the nucleotide sequence set forth in SEQ ID NO: 4-7.

The “direct repeat (DR) sequence” in the present disclosure refers to a DNA coding sequence in a CRISPR locus, and when described at the RNA level, each T should be understood as representing a U.

To reduce off-target interactions, such as reducing an interaction between a specific guide sequence and a low-complementarity target sequence, a mutation may be introduced into the CRISPR system so that it can distinguish between the target sequence and off-target sequences with greater than 80%, 85%, 90%, or 95% complementarity. In some embodiments, the complementarity is between 80% and 95%, for example, about 83%, 84%, 85%, 86%, 87%, 88%, 89%, 90%, 91%, 92%, 93%, 94%, or 95% (e.g., distinguishing a target with 18 nucleotides from an off-target with 1, 2, or 3 mismatched nucleotides). Therefore, in some embodiments, the complementarity between a specific guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the complementarity is 100%.

It is known in the art that if a complementarity is sufficient for functionality, complete complementarity is not required. Cutting efficiency may be modulated by introducing mismatches, for example by introducing one or more mismatches, such as 1 or 2 mismatches between a specific guide sequence and a target sequence. If a mismatch (e.g., double mismatch) is located closer to a center (i.e., not at the 3′ or 5′ end), the mismatch has a greater effect on the cleavage efficiency. Therefore, the cleavage efficiency may be regulated by introducing one or more mismatches along the specific guide sequence. For example, if less than 100% target cleavage is desired, one or two mismatches between the guide sequence and the target sequence may be introduced into a spacer sequence.

Chemical modifications may be introduced to the phosphate backbone, sugars and/or bases of the guide RNA. Backbone modifications (e.g., phosphorothioate modifications) alter the charge on the phosphate backbone and aid in the delivery of the polynucleotide and resistance to nucleases (see, e.g., Eckstein, Nucl. Acid Ther., 24, pp. 374-387, 2014). Sugar modifications, such as 2′-O-methyl (2′-OMe), 2′-fluoro (2′-F), or locked nucleic acid (LNA) modifications, may enhance base pairing and nuclease resistance (see, e.g., Allerson, J. Med. Chem. 48.4:901-904, 2005). Sugar modifications may also include 2′-deoxy modifications. Chemically modified bases, such as 2-thiouridine or N6-methyladenosine, may strengthen or weaken base pairing (see, e.g., Bramsen, Front. Genet., 2012 Aug. 20; 3:154). In addition, the guide RNA may be conjugated at the 5′ and/or 3′ ends with various functional components, including fluorescent dyes, polyethylene glycol (PEG), or proteins. The 5′ end of the guide RNA may have one or more additional nucleotides attached to the guide sequence. Non-limiting examples include two additional guanine nucleotides at the 5′ end, which may enhance targeting specificity.

In some embodiments, modification of the guide RNA with 2′-OMe improves nuclease resistance, which alters a binding energy of Watson-Crick base pairing. In addition, the 2′-OMe modification may affect the interaction of the polynucleotide with transfection reagents, proteins, or any other molecule in the cell. Effects of these modifications may be empirically tested and obtained.

In some embodiments, the guide RNA comprises one or more phosphorothioate modifications. In some embodiments, to enhance base pairing and/or increase nuclease resistance, the guide RNA comprises one or more lock nucleic acids. In some embodiments, one or more ribonucleotides of the guide RNA may be substituted with deoxyribonucleotides.

### Polynucleotide

The present disclosure also provides an isolated polynucleotide encoding the Cas9 protein, the fusion protein, or a protein portion of the conjugate described in any of the embodiments of the present disclosure.

In some embodiments, the isolated polynucleotide comprises (i) the nucleotide sequence set forth in SEQ ID NO: 2; (ii) a nucleotide sequence having one or more nucleotides deleted, added, and/or substituted relative to SEQ ID NO: 2; (iii) a polynucleotide having at least 40%, at least 50%, at least 60%, at least 70%, at least 80%, at least 85%, at least 90%, at least 95%, at least 96%, at least 97%, at least 98%, at least 99%, or at least 99.5% sequence identity to SEQ ID NO: 2; (iv) a polynucleotide that may hybridize under stringent conditions with any polynucleotide of (i)-(iii) or a complementary sequence thereof; (v) the complementary sequence of any polynucleotide of (i)-(iii). Degenerate sequences of the above polynucleotides may also be used/included.

In some embodiments, the polynucleotide is a native sequence (non-altered sequence), a mutant sequence, or a codon-optimized sequence.

In some embodiments, the polynucleotide is codon-optimized for expression in prokaryotes. In some embodiments, the polynucleotide is codon-optimized for expression in eukaryotes, such as human or human cells.

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the Cas9 protein, derivatives thereof, or functional fragments thereof). In some embodiments, the mRNA is capped, polyadenylated, modified with 5-methylcytosine, pseudouridylated, or any combination of the above.

In some embodiments, the nucleic acid (e.g., DNA) is operably linked to a regulatory element (e.g., a promoter) to control expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters may be those known in the art, including but not limited to pol I promoters, pol II promoters, pol III promoters, T7 promoters, U6 promoters, H1 promoters, retroviral Moloney murine leukemia virus (MLV) LTR promoters, cytomegalovirus (CMV) promoters, SV40 promoters, dihydrofolate reductase promoters, and β-actin promoters. For example, a U6 promoter may be used to regulate the molecular expression of the guide RNA described in the present disclosure.

In some embodiments, one or more nucleic acids are present in a vector (e.g., a viral vector or a bacteriophage). The vector may be a cloning vector or an expression vector. The vector may be a plasmid, a bacteriophage, a phagemid, or the like. The vector may include one or more regulatory elements, allowing the vector to replicate in a target cell (e.g., a bacterial cell or a mammalian cell). In some embodiments, the vector contains a nucleic acid encoding a single component of the CRISPR-associated (Cas) system of the present disclosure. In some embodiments, the vector contains multiple nucleic acids, each encoding a component of the CRISPR-associated (Cas) system of the present disclosure.

### Recombinant Vector

The present disclosure further provides a recombinant vector comprising (a) any one of the polynucleotides

### Example 1: Screening of CasRfg.3 Protein

Through extensive research and experimental validation, the inventors of the present disclosure identified a novel Cas9 nuclease (referred to as CasRfg.3 or Ca2) from a large pool of candidate proteins. This new Cas9 nuclease has a sequence that is significantly different from known Cas9 nucleases.

The genomic sequence of the CasRfg.3 protein is derived from the source shown in Table 1.

TABLE 1
Source of the genomic sequence of the CasRfg.3 protein

| | | | Corresponding Coding | |
|---|---|---|---|---|
| | | | Sequence Location in | Species Source (as |
| Protein | Database | Genomic ID | Genome | annotated by database) |
| CasRfg.3 | NCBI | GCA_902786545.1 | GCA_902786545.1: CAD | species: Uncultured |
| protein | Genbank | | ACU010000005.1: 49546: | Anaerovibrio sp. |
| | | | 52686+ | (Firmicutes) |
| | | | | Isolate: RUG13183 |

The sequence of the CasRfg.3 protein is shown
below (SEQ ID NO: 1, 1046 aa):
MKRKIGIGLDVGIGSVGFAVLSYDKVYDARIEQVGVRLFDSGEEPKTKV
SKNQGRRQYRAGRRLIRRRYHRKERAKRFIERIGLLSADKIKEWQEVNG
NQNIYYVRFRGLSEKLTPQEIADCVIHFCNHRGYREFYEDDVADEKEAG
KIKTALSRFDEKMAAGKYVSVADMILHDNEFSTNTQFPDFHNHKGDDDE
KYFLIKRAALRDELRAILRKQQEFYEQLTEHNIDFLCDDIVFVQRDFED
GPGDKTDKKRKFMGFLDSIGCCMFYKEEVRGYRSTVIADIYSLVNGLSK
MMYVDSTTGEITFLPEAADKIIDFALKNASITEKDIKEILKKYNLTLIK
AEKLEENIPQTIKTLKILKKILDDSGYSYDELIKEEQFDFDRPSKLHEL
CSLLASNITPKRRRKALEKAGWNKALQEQTRRIHFGGTSNVCYRYMLEA
IDAFRHGELYGNFQARRNKEQLTDEAENTEKVRLLPPFTKEMDEDVVKN
VVVFKAINETRKIINALIGKYGSPAYINIEVADELGHSIETRRKMTKAN
NEKMKKKEAISTKLVELGLRKEGEVSGKDIARYRLWEQQDGIDLYTGEN
IPEADVLSGQYDIDHIIPFSLILDDTLNNKVLTGMGSNRQAKSNKAPRE
YLSDKAELEFIKRVNILLKKKIISKKKYKYLMVKNLRDSKLLDEWKSRN
INDTRYISRFLVNYLNNMLLFNSDKKKNVYAINGAITSKMRKLWLNKRT
WGTPEKNRENNLHHAADAIVIANLTPAAVELASDNLKLONIFRQNGKRV
TEEYDNYLDKAVRKMEKYYHLNEELAKNLLVRKDRIPSMVRSLREETDK
RLVDTSLEEFNMVTPEAFRKNLEQYYNDSDFVASIQMPLVSYKQSKRFS
GSFTKDNPIKKKDKEDSSSVKMDSLGNENILDAKSYYCLEVYSTKDNKT
ALRGLRYVDFKLKNKKMFINVPNPDNYDKHIMYLFKNDYIVVYNKKGEE
RIKGYYTGIKNIKANRFYLISNNDAVRKDFTLSKDDTIKKYHIDILGQI
GGEVKCSAPFLSITEKE

Identification revealed that in the above protein, the amino acid residues at positions 1-52 (Ruvc_I), 473-530 (Ruvc_II), and 686-834 (Ruvc_III) (underlined) correspond to the RuvC nuclease domains; the amino acid residues at positions 531-685 (italicized) correspond to the HNH nuclease domain. The HNH nuclease domain cleaves the complementary strand, while the RuvC nuclease domain cleaves the non-complementary strand, producing a double-strand break (DSB) with specificity.

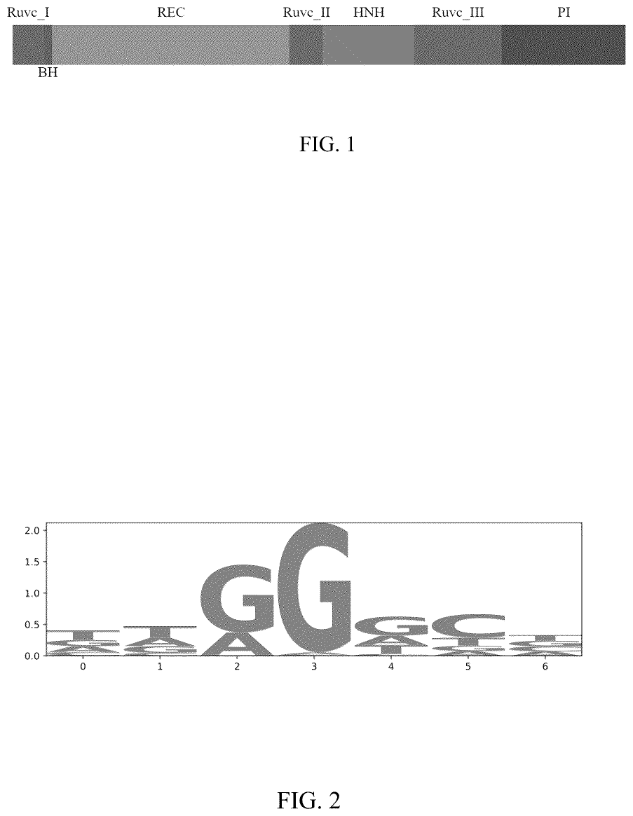

A domain distribution of the CasRfg.3 protein is shown in FIG. 1.

The native (wild-type) DNA encoding sequence for the CasRfg.3 protein is as follows
(SEQ ID NO: 2, 3141 bp):
atgaaaagaaaaattggaatcgggttggatgttggtataggttctgtaggttttgctgtattgtcgtatgataaggtatatgatgccc
gtatcgaacaggtaggggtccggctttttgattctggggaagaacccaaaacaaaagtcagtaagaaccaggggagacggcaatataggg
ctggtcgtagattgattcggcgtagataccatcgtaaagagcgggcaaagagatttattgagcggataggtttattaagtgcggacaaaatta
aagaatggcaggaagtaaatggcaatcagaatatttactatgtgagatttaggggattgtcggaaaagctcaccccacaagaaattgccgatt
gcgttatccatttttgcaaccatagaggttatcgtgaattctatgaagatgatgttgcggatgaaaaggaagctggcaagataaagaccgccct
atccagatttgatgaaaaaatggcagctggtaagtatgtatccgttgccgatatgattttgcatgataatgaattttctacaaacacccagtttc
cagatttccataatcataaaggtgatgacgacgaaaaatattttcttattaaacgggcagcgttaagggatgaacttagggcaatactccggaagc
aacaggaattctatgaacagttgacggaacataatattgatttcttatgtgacgacatagtttttgttcagcgggattttgaggatggacccggg
gataaaactgataaaaagcgtaagtttatgggttttttggatagcataggctgctgtatgttttataaggaagaagttagaggctataggtccac
agtaattgcagatatttattccttggtaaacggtttgtccaagatgatgtatgttgacagcaccactggtgaaataacatttttaccggaagctgc
tgacaaaattattgattttgccctaaaaaatgcatccattactgaaaaagatatcaaagaaatactgaagaaatacaatttgacccttataaaggc
tgaaaagctagaggaaaacataccgcagactataaagactctgaagatattgaaaaagatattggatgatagtgggtatagttatgatgaattg
atcaaggaagaacaatttgattttgataggccatccaaactccatgaattatgtagtttgttagccagcaatattaccccaaaacgcagaagaa
aagcattggaaaaagctggctggaataaagcattgcaagaacaaaccaggagaatacactttggtggaacatcaaatgtatgctatcgctac
atgctagaagctattgatgccttcagacatggtgaattatatggtaatttccaagcccgacgaaataaggaacagcttacagatgaagcagaa
aatacggagaaggtaagattattaccaccgttcacaaaagaaatggatgaagatgtggtaaagaatgtagtggttttcaaggcaatcaatgaa
acccgcaagataatcaatgcccttataggcaagtacggttcgccggcatatatcaatattgaggtggctgatgaactggggcacagtattgaa
accaggcggaaaatgaccaaagccaacaacgaaaaaatgaaaaaaaaggaggctattagtacaaagctggttgaactgggacttcgtaaa
gagggggaagtatctggtaaagatatagcgcgttatcgtctatgggagcagcaggatgggatagatttatatacgggggaaaatattccaga
agcggatgtgctgagtgggcaatatgatattgatcacattatcccattttcgctgatattggatgataccctaaataataaggtactgactggtat
ggggagcaatcgtcaggcaaaatccaataaagctcctagagaatatttatctgataaggctgaattggagtttataaagagagtaaatatctta
ctgaagaaaaaaataatctcaaagaaaaagtacaagtacttgatggtaaagaaccttcgtgacagtaaactgctggatgagtggaagtcaag
aaatattaacgacaccagatatataagtcgttttttagtgaactatctaaataatatgttgctatttaacagcgataaaaagaaaaatgtatatgc
aatcaatggtgccattacatccaaaatgcgaaaactgtggcttaataagcgaacctggggaactccggaaaagaacagggaaaataatttgc
atcatgcagctgatgctatagttattgccaatcttactccagctgcagtggaactggccagtgataaccttaaactccagaatattttccgtcaaa
atggcaagcgagtgacggaggaatatgataattatcttgataaggcagttcgcaagatggagaagtattatcacttaaatgaagaattggcca
agaatctgctggtacgaaaggatcgtattccatccatggtaaggtcattgcgggaggaaacggataagagattggtggatacaagtcttgag
gaattcaatatggttacaccagaagcattccgcaaaaatctagagcagtattataatgactctgattttgttgcttccatacagatgccgttggta
tcctataagcagtccaagcggttttctggaagctttactaaggataatcctataaagaaaaaagataaagaagactcatcaagtgtaaaaatgg
actctttaggcaatgaaaatattttggatgccaaatcgtattactgtttggaggtgtattccacaaaagataataagacggctttgcgcggcctta
ggtatgttgattttaaattgaaaaacaaaaagatgtttatcaatgttcctaatccagataattatgataagcatattatgtatttgtttaagaatg
attatattgtggtgtacaacaaaaaaggagaggaaagaattaaaggttattatacaggaataaaaaacatcaaagctaatagattttatttgataa
gtaacaatgatgcggtgcgaaaagattttacattatctaaagatgatactataaaaaaatatcacattgatattcttgggcaaattggtggtgagg
taaaatgttccgctccattcttgtcaataacggagaaagaataa
The codon-optimized DNA encoding sequence for the CasRfg.3 protein is as follows
(SEQ ID NO: 10):
atgaagagaaagatcggcatcggcctggacgtgggcatcggcagcgtgggcttcgccgtgctgagctacgacaaggtgtac
gacgccagaatcgagcaggtgggcgtgagactgttcgacagcggcgaggagcccaagaccaaggtgagcaagaaccagggcagaag
acagtacagagccggcagaagactgatcagaagaagataccacagaaaggagagagccaagagattcatcgagagaatcggcctgctg
agcgccgacaagatcaaggagtggcaggaggtgaacggcaaccagaacatctactacgtgagattcagaggcctgagcgagaagctga
ccccccaggagatcgccgactgcgtgatccacttctgcaaccacagaggctacagagagttctacgaggacgacgtggccgacgagaag
gaggccggcaagatcaagaccgccctgagcagattcgacgagaagatggccgccggcaagtacgtgagcgtggccgacatgatcctgc
acgacaacgagttcagcaccaacacccagttccccgacttccacaaccacaagggcgacgacgacgagaagtacttcctgatcaagaga
gccgccctgagagacgagctgagagccatcctgagaaagcagcaggagttctacgagcagctgaccgagcacaacatcgacttcctgtg
cgacgacatcgtgttcgtgcagagagacttcgaggacggccccggcgacaagaccgacaagaagagaaagttcatgggcttcctggaca
gcatcggctgctgcatgttctacaaggaggaggtgagaggctacagaagcaccgtgatcgccgacatctacagcctggtgaacggcctga
gcaagatgatgtacgtggacagcaccaccggcgagatcaccttcctgcccgaggccgccgacaagatcatcgacttcgccctgaagaac
gccagcatcaccgagaaggacatcaaggagatcctgaagaagtacaacctgaccctgatcaaggccgagaagctggaggagaacatcc
cccagaccatcaagaccctgaagatcctgaagaagatcctggacgacagcggctacagctacgacgagctgatcaaggaggagcagttc
gacttcgacagacccagcaagctgcacgagctgtgcagcctgctggccagcaacatcacccccaagagaagaagaaaggccctggaga
aggccggctggaacaaggccctgcaggagcagaccagaagaatccacttcggcggcaccagcaacgtgtgctacagatacatgctgga
ggccatcgacgccttcagacacggcgagctgtacggcaacttccaggccagaagaaacaaggagcagctgaccgacgaggccgagaa
caccgagaaggtgagactgctgccccccttcaccaaggagatggacgaggacgtggtgaagaacgtggtggtgttcaaggccatcaacg
agacaagaaagatcatcaacgccctgatcggcaagtacggcagccccgcctacatcaacatcgaggtggccgacgagctgggccacag
catcgagacaagaagaaagatgaccaaggccaacaacgagaagatgaagaagaaggaggccatcagcaccaagctggtggagctgg
gcctgagaaaggagggcgaggtgagcggcaaggacatcgccagatacagactgtgggagcagcaggacggcatcgacctgtacaccg
gcgagaacatccccgaggccgacgtgctgagcggccagtacgacatcgaccacatcatccccttcagcctgatcctggacgacaccctg
aacaacaaggtgctgaccggcatgggcagcaacagacaggccaagagcaacaaggcccccagagagtacctgagcgacaaggccga
gctggagttcatcaagagagtgaacatcctgctgaagaagaagatcatcagcaagaagaagtacaagtacctgatggtgaagaacctgag
agacagcaagctgctggacgagtggaagagcagaaacatcaacgacaccagatacatcagcagattcctggtgaactacctgaacaacat
gctgctgttcaacagcgacaagaagaagaacgtgtacgccatcaacggcgccatcaccagcaagatgagaaagctgtggctgaacaaga
gaacctggggcacccccgagaagaacagagagaacaacctgcaccacgccgccgacgccatcgtgatcgccaacctgacccccgccg
ccgtggagctggccagcgacaacctgaagctgcagaacatcttcagacagaacggcaagagagtgaccgaggagtacgacaactacct
ggacaaggccgtgagaaagatggagaagtactaccacctgaacgaggagctggccaagaacctgctggtgagaaaggacagaatcccc
agcatggtgagaagcctgagagaggagacagacaagagactggtggacaccagcctggaggagttcaacatggtgacccccgaggcct
tcagaaagaacctggagcagtactacaacgacagcgacttcgtggccagcatccagatgcccctggtgagctacaagcagagcaagaga
ttcagcggcagcttcaccaaggacaaccccatcaagaagaaggacaaggaggacagcagcagcgtgaagatggacagcctgggcaac
gagaacatcctggacgccaagagctactactgcctggaggtgtacagcaccaaggacaacaagaccgccctgagaggcctgagatacgt
ggacttcaagctgaagaacaagaagatgttcatcaacgtgcccaaccccgacaactacgacaagcacatcatgtacctgttcaagaacgac
tacatcgtggtgtacaacaagaagggcgaggagagaatcaagggctactacaccggcatcaagaacatcaaggccaacagattctacctg
atcagcaacaacgacgccgtgagaaaggacttcaccctgagcaaggacgacaccatcaagaagtaccacatcgacatcctgggccagat
cggcggcgaggtgaagtgcagcgcccccttcctgagcatcaccgagaaggag

According to the bacterial genomic sequence in Table 1, the DR sequence and tracrRNA sequence corresponding to the crRNA for the CasRfg.3 protein are as follows:

DR sequence (SEQ ID NO: 11):
guuuuaguucuauguuggauauugauaaacugauac;
tracrRNA sequence (SEQ ID NO: 12):
uugucaguuuaucaauauccaacaauaguucuaagauaaggccuuaugu
gccguaggguauagcgguaucccgaacaauuccgcugcuuugaguauua
agcugcuacaucauguagcagc.

### Example 2: Preparation of CasRfg.3 Protein

### 1. Vector Construction

A pET28a vector plasmid was digested with BamHI and XhoI, followed by agarose gel electrophoresis to recover a linearized vector through gel extraction. A DNA sequence encoding the CasRfg.3 protein, prepared by conventional methods, was inserted into a cloning region of the pET28a vector via homologous recombination to construct a recombinant vector CasRfg.3-pET28a. The reaction mixture was used to transform Stb13 competent cells, which were then plated on LB agar containing kanamycin sulfate and cultured overnight at 37° C. Clones were picked for sequencing verification. The sequence of the constructed recombinant vector CasRfg.3-pET28a is presented as SEQ ID NO: 13.

Positive clones with correct sequence were selected, and a single clone was picked for overnight culture. After plasmid extraction, the plasmid was transformed into the expression strain Rosetta (DE3). The transformed cells were plated onto a LB agar plate containing kanamycin sulfate (resistant) and cultured overnight at 37° C.

### 2. Protein Expression

A single colony was inoculated into 5 ml of LB medium containing kanamycin sulfate (resistant) and cultured overnight at 37° C.

The culture was then transferred at a ratio of 1:100 into 500 ml of LB medium supplemented with kanamycin sulfate (resistant). The culture was incubated at 37° C. with shaking at 220 rpm until an OD of 0.6 was reached. IPTG was added to a final concentration of 0.2 mM, and the culture was induced at 16° C. for 24 hours.

After washing the cells with 15 mL of PBS and centrifuging to collect the cells, the cells were lysed by sonication in lysis buffer. The supernatant, containing a recombinant protein, was obtained by centrifuging at 10,000 g for 30 minutes. The supernatant was filtered through a 0.45 μm filter membrane and then applied to a column for purification.

### 3. Protein Purification

The recombinant CasRfg.3 protein consists of 1135 amino acids (with the native CasRfg.3 protein composed of 1046 amino acids) and is structured as His tag-NLS-CasRfg.3-SV40 NLS-nucleoplasmin NLS. The CasRfg.3 recombinant protein was purified using an N-terminal His tag through immobilized metal affinity chromatography (IMAC) using Ni Sepharose 6 Fast Flow (Cytiva), followed by ion exchange chromatography (Cytiva HiTrap™ Q FF) and size exclusion chromatography (Cytiva Superdex™ 200 Increase 10/300 GL). The purified recombinant protein was analyzed by SDS-PAGE, displaying a single band.

### Example 3: Determination of PAM Sequence of CasRfg.3 Protein

In this example, a single guide RNA (sgRNA) containing a specific guide sequence is combined with the CasRfg.3 recombinant protein purified in Embodiment 2 to cleave an in vitro substrate (containing a spacer sequence and a 7 nt random sequence). After incubation at 37° C., purification, library construction, and next-generation sequencing (NGS) are performed to analyze the PAM sequence of CasRfg.3. The specific operations are as follows:

### A. In Vitro Cleavage of a Substrate by CasRfg.3 Protein

The sequence of the substrate cleaved by
CasRfg.3 protein in vitro is (SEQ ID NO: 14):
ggagttcagacgtgtgctcttccgatctcagcacaaaaggaaactcaccc
taactgtaaagtaattgtgtgttttgagactataaatatgcatgcgagaa
aagccttgtttgccaccatgGAACGGCTCGGAGATCATCATTGCGNNNNN
NNgtgagcaagggcgaggagctgttcaccggggtggtgcccatcctggtc
gagctggacggcgacgtaaacggccacaagttcagcgtgtccggcagatc
ggaagagcacacgtctgaactcc

In the sequence, N represents any one of A, T, C, or G.

Double-stranded DNA containing the above sequence is prepared using conventional PCR amplification methods and used as an in vitro cleavage substrate.

The cleavage substrate is sent to a sequencing company for PCR-free library construction and NGS sequencing. The complexity and abundance of the PAM library, consisting of 7 nt random sequences, are analyzed. The results are as follows.

The composition of the four bases A, T, G, and C is basically consistent. The PAM library consisting of 7 nt random sequences includes different combinations totaling 4⁷=16,384, all of which were detected. The complexity and abundance of the PAM library are qualified.

B. Preparation of sgRNA in Conjunction with CasRfg.3

SgRNA containing the specific guide sequence (CasRfg.3-sgRNA) is synthesized in vitro at 37° C. in a system containing T7 RNA polymerase, four ribonucleoside triphosphates, and a DNA template with a T7 promoter. The transcription product is purified using the Ribo™ RNAmax-T7 In vitro Transcription Kit purification reagent. The sgRNA sequence is as follows (SEQ ID NO: 15):

5′-GAACGGCUCGGAGAUCAUCAUUGCGguuuuaguucuauguuggauau
ugauaaacugauacgaaauugucaguuuaucaauauccaacaauaguucu
aagauaaggccuuaugugccguaggguauaggguauccegaacaauucgc
ugcuuugaguauuaagcugcuacaucauguagcagc-3′.

The capitalized bases represent the specific guide sequence of the sgRNA.

### C. NGS Library Construction and PAM Analysis

A reaction system containing the CasRfg.3 protein, the sgRNA, the in vitro cleavage substrate, and a buffer is prepared to react at 37° C. for 3 hours, followed by 75° C. for 15 minutes. As shown in Table 2.

TABLE 2

| Component | Amount (μl) |
|---|---|
| 10xCut Buffer(200 mM HEPES, 1M NaCl, 50 mM | 5 |
| MgCl₂, 1 mM EDTA) | |
| Cleavage substrate (59.5 ng/μl) | 36.5 |
| CasRfg.3 sgRNA(340.3 ng/μl) | 8 |
| CasRfg.3protein (10 mg/ml) | 0.5 |

The cleaved fragments are subjected to a ligation reaction to add a biotin-labeled adapter (Adapter1) to the ends of the cleavage sites. The biotin-labeled adapter-containing cleavage products are specifically bound to streptavidin-coated magnetic beads (Beads) for purification. The specifically purified fragments are then amplified by PCR and subjected to NGS sequencing.

The NGS library is constructed through the following operations:

a. Add 68.16 μl of SPRISelect Beads (Beckman COULTER, B23318) to a reaction product, mix thoroughly, and incubate at room temperature for 5 minutes. Transfer the product to a magnetic stand and let it adhere for 5 minutes. Transfer the supernatant to a new 1.5 ml tube; then add 34.08 μl of SPRISelect Beads (Beckman COULTER, B23318), mix thoroughly, and incubate at room temperature for 5 minutes. Transfer the product to a magnetic stand and let it adhere for 5 minutes. Discard the supernatant, wash the product twice with 85% ethanol, air-dry at room temperature for 10 minutes, and elute with 50 μl of ddH2O.

b. Perform end repair and add dA-tailing (NEB, E6053L) to the product using the system shown in Table 3, and perform the reaction at 37° C. for 30 minutes.

TABLE 3
Addition of dA-tailing to Ca2 cleavage product

| Component | Amount (μl) |
|---|---|
| Ca2 cleavage product | 42 |
| dA-tailing reaction buffer | 5 |
| Klenow Fragment | 3 |
| ddH₂O | Up to 50 |

c. Add Adapter 1 (annealed from upstream primer: 5′Biosg/gttgacatgctggattgagacttcctacactctttccctacacgacgctcttccgatc*t (SEQ ID NO: 16) and downstream primer: gatcggaagagcgtcgtgtagggaaagatgtaggaagtctcaatccagcatgtcaac (SEQ ID NO: 17)) according to the system in Table 4, and incubate at 20° C. for 30 minutes followed by an overnight reaction at 16° C.

TABLE 4
Addition of Adapter 1 reaction system

| Component | Amount (μl) |
|---|---|
| dA-tailed DNA | 38 |
| Adapter 1 | 2 |
| T4 DNA Ligase buffer(10x) | 5 |
| NEB Quick Ligase | 5 |
| ddH₂O | Up to 50 |

The above reaction products were purified using SPRISelect Beads after overnight reaction.

d. Product purification using Streptavidin-labeled magnetic beads Dynabeads® M-280 Streptavidin (Invitrogen, 11206D).

e. Recover PCR

Primers from Table 5 were designed and Recover PCR was performed using Q5® Hot Start High-Fidelity 2× Master Mix (NEB, M0494L) according to the system in Table 6 as well as the reaction program in Table 7.

TABLE 5
Recover PCR primers

| Primer ID | Sequence |
|---|---|
| Recovery PCR | ggagttcagacgtgtgctc (SEQ |
| Forward | ID NO: 18) |
| Recovery PCR | gttgacatgctggattgagacttc |
| Reverse | (SEQ ID NO: 19) |

TABLE 6
Recover PCR reaction system

| Component | Amount (μl) |
|---|---|
| Streptavidin magnetic beads purified product | 22.5 |
| Recovery PCR Forward(10 uM) | 2.5 |
| Recovery PCR Reverse(10 uM) | 2.5 |
| Q5 Hot-Start 2x Master Mix | 22.5 |
| ddH2O | Up to 50 |

TABLE 7
Recover PCR reaction program

| Reaction Temperature | Duration | | Cycles |
|---|---|---|---|
| 98° C. | 2 | min | 1 |
| 98° C. | 10 | sec | 12 |
| 61° C. | 30 | sec | |
| 72° C. | 2 | min | |
| 72° C. | 2 | min | 1 |

| | |
|---|---|
| 4° C. | ∞ |

f. Recover PCR product was transferred to a magnetic rack, adsorbed for 5 min, supernatant was transferred to a new 1.5 ml centrifuge tube, 3 μl of Recovery PCR product was taken and diluted by adding 148.5 μl of ddH2O.

g. Index PCR

Index PCR was performed using the primers from Table 8 according to the system of Table 9 as well as the reaction program of Table 10.

TABLE 8
Index PCR primers

| Primer | Sequence |
|---|---|
| IF501 | aatgatacggcgaccaccgagatctacactatagcct |
| | acactctttccctacacgacg(SEQ ID NO: 20) |
| IR701 | caagcagaagacggcatacgagatcgagtaatgtgac |
| | tggagttcagacgtgtgctc(SEQ ID NO: 21) |

TABLE 9
Index PCR reaction system

| Component | Amount (μl) |
|---|---|
| Recovery PCR diluted product | 12 |
| IF501(10 uM) | 4 |
| IR 701(10 uM) | 4 |
| Q5 Hot-Start 2x Master Mix | 20 |
| Total | 40 |

TABLE 10
Index PCR reaction program

| Reaction Temperature | Duration | | Cycles |
|---|---|---|---|
| 98° C. | 2 | min | 1 |
| 98° C. | 10 | sec | 12 |
| 60° C. | 30 | sec | |
| 72° C. | 2 | min | |
| 72° C. | 2 | min | 1 |

| | |
|---|---|
| 4° C. | ∞ |

h. The Index PCR product was purified by adding 0.7× SPRISelect Beads, eluted by adding 38 μl ddH2O, and concentration was determined by using Qubit at 22.4 ng/μl, which met the requirements for testing and was sent to NGS for sequencing.

i. Analysis of NGS results: Using the NGS data and following the method described in the reference (A compact Cas9 ortholog from Staphylococcus auricularis (SauriCas9) expands the DNA targeting scope. PLOS Biology, 2020, 18 (3), e3000686), the PAM sequence for CasRfg.3 was analyzed using WebLogo software, as shown in FIG. 2.

Thus, the PAM sequence of CasRfg.3 includes: 5′-DDRGDNN-3′ (D is A, G or T; R is A or G; N is A, C, G or T). Specifically, the PAM sequence includes: 5′-ATAGGCT-3′, 5′-AAGGCT-3′, 5′-AGGGTCG-3′, 5′-GAGGTCC-3′, 5′-TTGGGCT-3′, 5′-TGGGGTA-3′, 5′-TGGGGGC-3′, 5′-GGGGGGA-3′, 5′-AGAGGAT-3′, 5′-AGGGTCC-3′, 5′-ATAGGGA-3′, 5′-GTAGAAT-3′, 5′-GGGGAAG-3′, 5′-TAAGGTT-3′, and 5′-TGGGTAG-3′.

The unique PAM of CasRfg.3 expands the range of targetable sequences.

### Example 4: Selection of Target Nucleic Acid Sequences in the Human Genome for Cleavage Validation Based on PAM Analysis Results

Target nucleic acid sequences in the human genome that are consistent with the PAM sequence identified for the CasRfg.3 protein in Example 3 above were selected for cleavage activity validation. The specific operations are as follows:

### A. Amplification of Cleaved Substrate

Based on the analyzed PAM sequences, two different fragments CasRfg.3-N2 and CasRfg.3-N4 with PAM sequences TTGGTCT and GAGGTCG, respectively, were selected from the human genome for cleavage testing.

### 1. Sequence Information of Fragments CasRfg.3-N2 and CasRfg.3-N4

>CasRfg.3-N2 sequence (SEQ ID NO: 22):
Gaatgggctatttctaagcacggcaggtaccgtcttccccttcccccgcc
aacacagctggcccaggacaagaaggtgcagagacaaacagggggcaccc
tgcctgtcctgctaggaactgcaaggcagcgtgccgcacacgctgcctgg
gccaatgccacccaggccaggagagggtttggggccagacaccagcccat
acccaagggtcccaggggatgtggggagaaggggaatccaccttttcctt
ccctcccacctcccaaATAACACACAGACAGCTCTGTTGGTCTgagaatg
atggacatttagacactggcgccaggtttgcgcctgaccggcgccacgca
ggggtgggcggagcaaagacacacaggtgggctacaggtgtcacacggca
ccagccagggcccggggtggctggggtgaggatgggtgtttggccagtga
ccaggagtcaggtcaagtccaggtggtcagtgccaggggctccaggaggg
gagggcagtgccataaccctcctggtgtccag
>CasRfg.3-N4 sequence (SEQ ID NO: 23):
ggcactgtgccctccagattcctcagagcctggtgccaggtgcacctccc
cagccctctcagcccctgagctctgccaccctgcccgggcttctgccctt
gggtcctggcctctcagggtctcctcagctctgcaaggaggggctgggag
caggtcccaggctagcagtgggctggaggcctcctgtgtggtctctacct
gtccatgCCGAGGGGTCACCTCTGGGTGAGGTCGcacccacccccctcac
ggggctgctttgccagaaggtctgttcccctcatggcctctgctactctt
tacacttagttcttcccaggaagggcccggtggctgcagataatgtttcc
catctgtggagccttcaagggctgggcccatgaggattctgctcccagct
tgctcttcctctccagcctgcacccaccaggccccagaatcctgaggatg
gagctgctcctgtcccctgaggtggtctaaacctctggggaggcactgga

The capitalized bases correspond to the target sequence; the underlined bases represent the PAM sequence.

### 2. Vector Construction

The CasRfg.3-N2 and CasRfg.3-N4 sequence fragments were amplified and ligated into the pEASY®-Blunt Zero Cloning (TransGen, Cat. No. CB501) vector. The ligation product was heat-shock transformed into E. coli 2T1 competent cells (Yingjun Bio, Cat. No. CC007). The transformed ligation product was verified by sequencing.

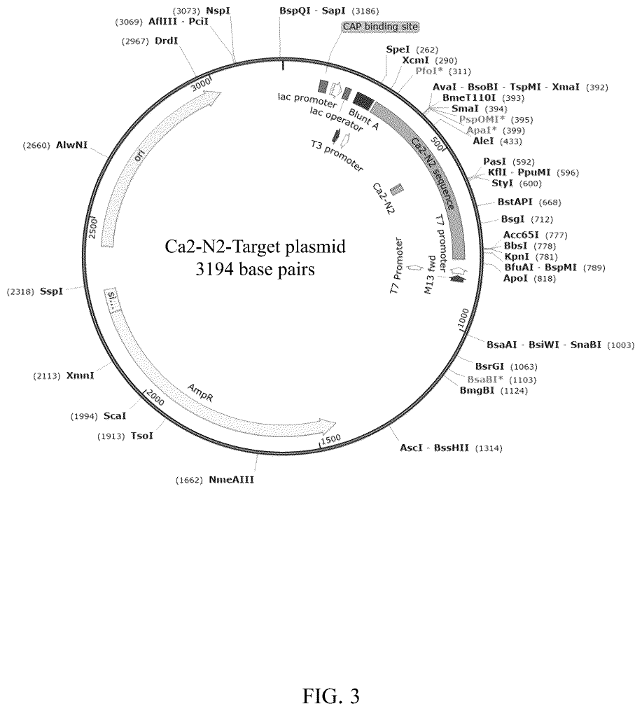

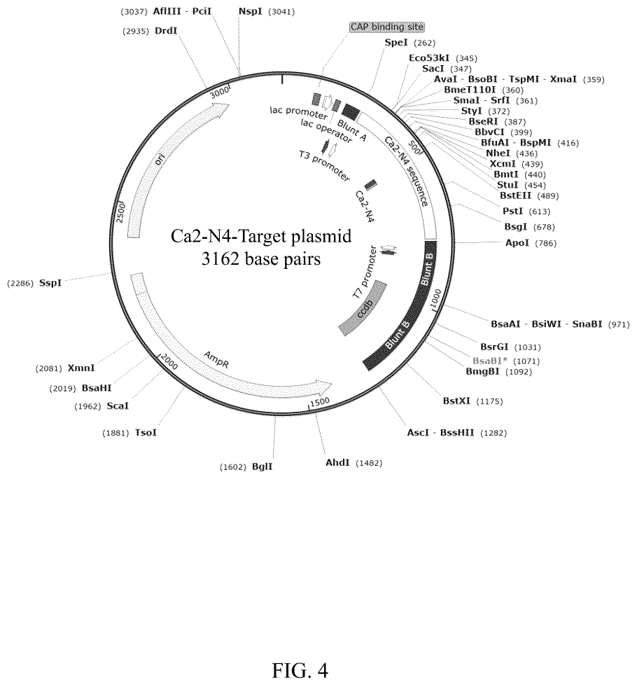

The constructed CasRfg.3-N2-Target plasmid is shown in FIG. 3 and SEQ ID NO: 24; CasRfg.3-N4-Target plasmid is shown in FIG. 4 and SEQ ID NO: 25.

The constructed CasRfg.3-N2-Target plasmid and CasRfg.3-N4-Target plasmid were linearized enzymatically using XmnI (NEB, R0194), and the linearization system is shown in Table 11.

TABLE 11

| Component | Amount (μl) |
|---|---|
| Ca2-N2-Target plasmid/Ca2-N4-Target plasmid | 10 μg |
| 10xCutSmart Buffer | 5 |
| XmnI(10 μ/μl) | 1 |
| ddH₂O | Up to 50 |

After completion of the reaction at 37° C. according to the above system, the product was purified using the Wizard® SV Gel and PCR Clean-Up System (Progema, A9282) and the concentration was determined by Nanodrop.

B. In Vitro Transcription of CasRfg.3-N2 Corresponding sgRNA and CasRfg.3-N4 Corresponding sgRNA

Two sgRNAs were designed for the target sequences of CasRfg.3-N2 and CasRfg.3-N4 and transcribed in vitro. The sgRNA sequences corresponding to CasRfg.3-N2 and CasRfg.3-N4 are as follows:

CasRfg.3-N2-sgRNA sequence (SEQ ID NO: 26):
5′-AUAACACACAGACAGCUCUGguuuuaguucuauguuggauauugaua
aacugauacgaaauugucaguuuaucaauauccaacaauaguucuaagau
aaggccuuaugugccguaggguauaggguaucccgaacaauuccgcugcu
uugaguauuaagcugcuacaucauguagcagc-3′
CasRfg.3-N4-sgRNA sequence (SEQ ID NO: 27):
5′-ccgaggggucaccucuggguguuuuaguucuauguuggauauugaua
aacugauacgaaauugucaguuuaucaauauccaacaauaguucuaagau
aaggccuuaugugccguaggguauaggguaucccgaacaauuccgcugcu
uugaguauuaagcugcuacaucauguagcagc-3′

The capitalized bases represent the guide sequence.

Transcription products were purified using purification reagents provided in the Ribo™ RNAmax-T7 In Vitro Transcription Kit, and RNA concentration was determined by Nanodrop for future use.

### C. In Vitro Cleavage Experiment

Reaction systems for cleavage were prepared according to Tables 12 and 13, reacted at 37° C., and inactivated at 75° C.

TABLE 12
In vitro cleavage reaction system for CasRfg.3-N2

| Component | Amount (μL) |
|---|---|
| 10xCut Buffer (200 mM HEPES, 1M NaCl, 50 mM | 5 |
| MgCl₂, 1 mM EDTA) | |
| CasRfg.3-N2-Target linearized plasmid (140.8 ng/μL) | 17 |
| CasRfg.3-N2-sgRNA(178.9 ng/μL) | 15 |
| Purified CasRfg.3 recombinant protein of Embodiment 2 | 0.5 |
| (10 mg/ml) | |
| ddH₂O | Up to 50 |

TABLE 13
In vitro cleavage reaction system for CasRfg.3-N4

| Component | Amount (μL) |
|---|---|
| 10xCut Buffer (200 mM HEPES, 1M NaCl, 50 mM | 5 |
| MgCl₂, 1 mM EDTA) | |
| CasRfg.3-N4-Target linearized plasmid (189.9 ng/μL) | 13 |
| CasRfg.3-N4-sgRNA(428.5 ng/μL) | 6.5 |
| Purified CasRfg.3 recombinant protein of Embodiment 2 | 0.5 |
| (10 mg/ml) | |
| ddH₂O | Up to 50 |

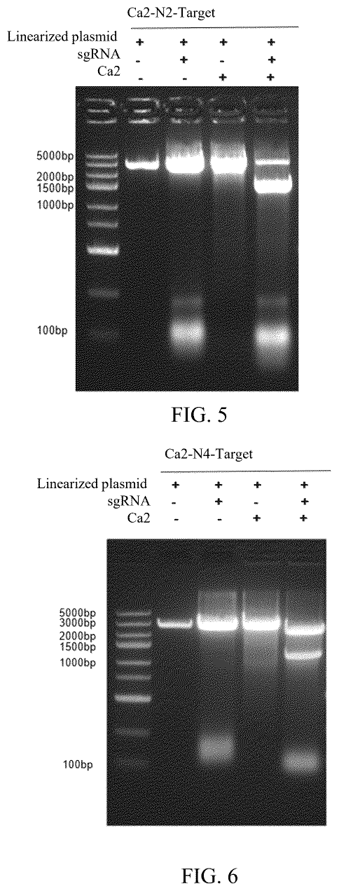

6 μL of loading buffer was added to the cleavage products, and 30 μL of the cleavage products was used for electrophoresis to assess the cleavage effect. Electrophoresis results are shown in FIGS. 5 and 6. The results demonstrate that the CasRfg.3 protein exhibited gRNA-guided nuclease activity with high cleavage efficiency.

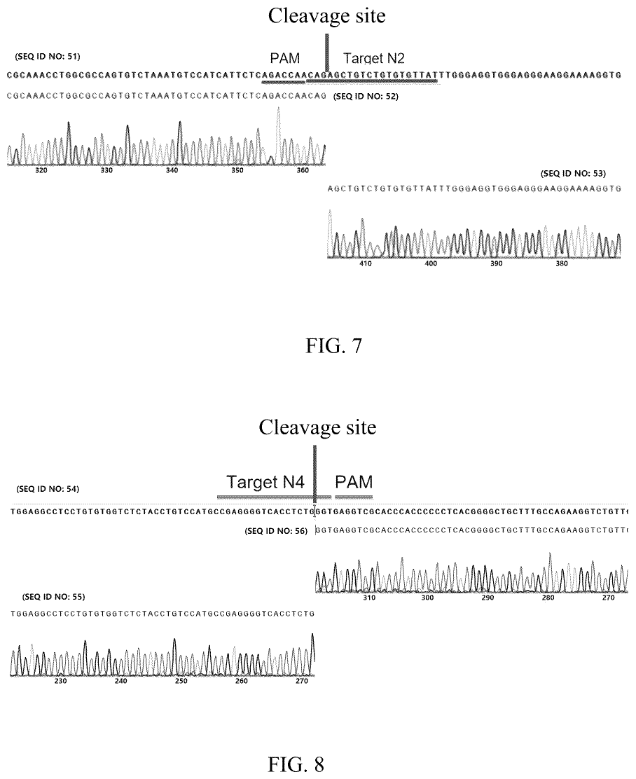

Fragments resulting from the cleavage were gel-extracted and sequenced. Sequencing results are shown in FIGS. 7 and 8, indicating cleavage activity and revealing the cleavage sites.

### Example 5: asRfg.3 Protein Cleavage Stability Experiments

A reaction system was prepared according to Table 14, and the cleavage effect was repeatedly tested at 37° C.

TABLE 14
In vitro cleavage reaction system for CasRfg.3-N2

| Component | Amount (μL) |
|---|---|
| 10xCut Buffer(200 mM HEPES, 1M NaCl, 50 mM | 5 |
| MgCl₂, 1 mM EDTA) | |
| Linearized CasRfg.3-N2-Target Plasmid of Embodiment 4 | 17 |
| (140.8 ng/μL) | |
| CasRfg.3-N2-sgRNA of Embodiment 4 (178.9 ng/μL) | 15 |
| Purified CasRfg.3 Recombinant Protein of Embodiment 2 | 0.5 |
| (10 mg/ml) | |
| ddH₂O | Up to 50 |

After inactivation at 75° C., 6 μL of loading buffer was added to the product. A 30 μL sample was taken for electrophoresis, and the results are shown in FIG. 9, indicating that DNA can be cleaved stably across multiple experiments.

### Example 6: Cleavage Effects of asRfg.3 Protein at Different Temperatures

A reaction system was prepared according to Table 15. The cleavage was conducted at 25° C., 30° C., 34.9° C., 37° C., 39.9° C., 45.4° C., 50° C., and 55° C. for the same duration, followed by inactivation at 75° C.

TABLE 15
In vitro cleavage reaction system for CasRfg.3-N2

| Component | Amount (μL) |
|---|---|
| 10xCut Buffer(200 mM HEPES, 1M NaCl, 50 mM | 5 |
| MgCl₂, 1 mM EDTA) | |
| CasRfg.3-N2-Target Plasmid of Embodiment 4 | 17 |
| (140.8 ng/μL) | |
| CasRfg.3-N2-sgRNA of Embodiment 4 (178.9 ng/μL) | 15 |
| CasRfg.3protein of Embodiment 2 (10 mg/ml) | 0.5 |
| ddH₂O | Up to 50 |

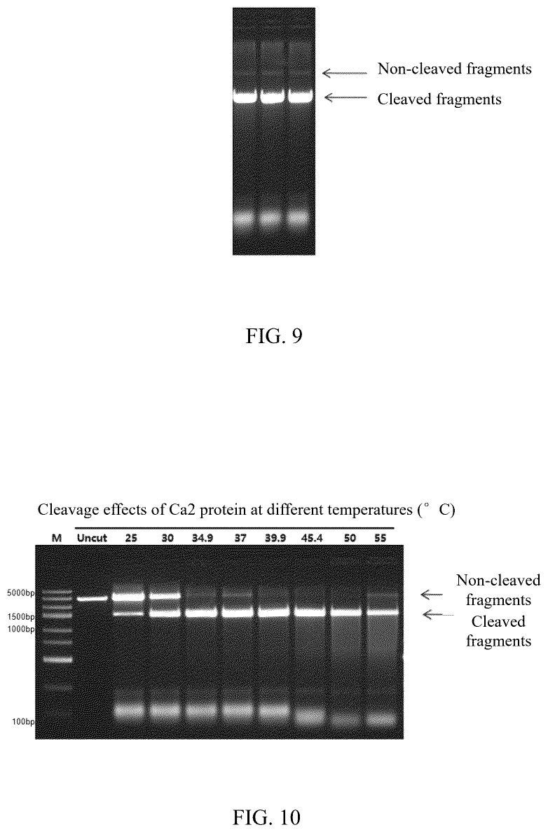

For each cleavage product, 6 μL of loading buffer was added and 30 μL was taken for electrophoresis to assess the cleavage effect. Electrophoresis results are shown in FIG. 10, indicating detectable cleavage activity from 25° C. to 55° C., with higher activity between approximately 35° C. to 55° C., and optimal cleavage activity observed around 50° C.

### Example 7: Modification of gRNA scaffold sequence

Previous examples have demonstrated that a single molecule sgRNA, obtained by linking the natural DR sequence and tracrRNA sequence corresponding to CasRfg.3, exhibits activity. The scaffold sequence for the sgRNA is referred to as CasRfg.3-sgRNA-scaffold.

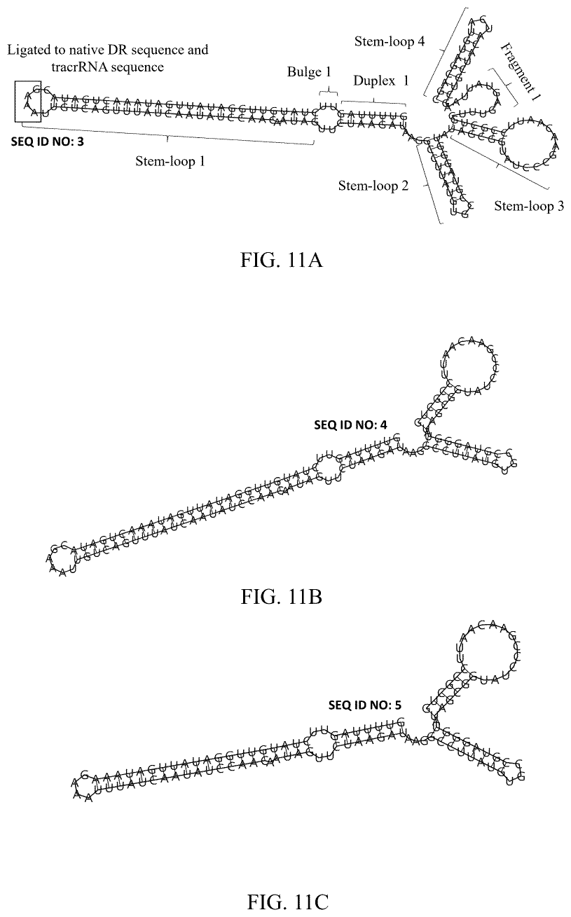

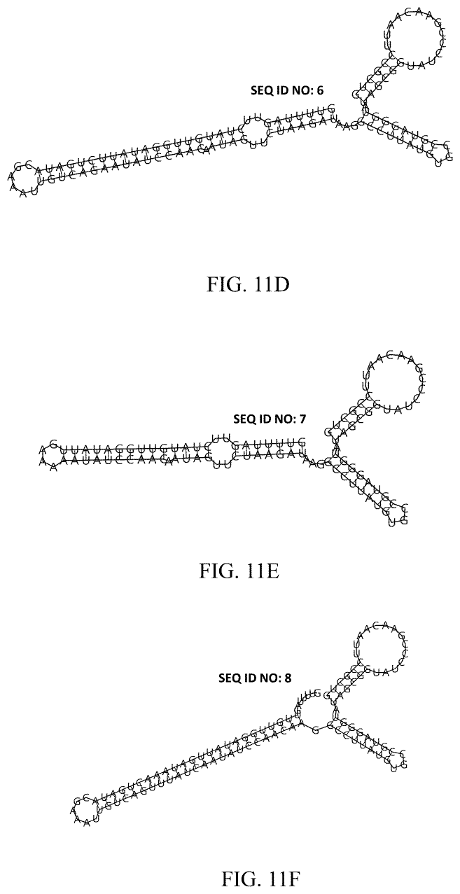

>CasRfg.3-sgRNA-scaffold (SEQ ID NO: 3,
160 nt; formed by linking natural DR and
tracrRNA sequences to create the sgRNA
scaffold; shown in FIG. 11A):
5′-GUUUUAGUUCUAUGUUGGAUAUUGAUAAACUGAUACGAAAUUGUCAG
UUUAUCAAUAUCCAACAAUAGUUCUAAGAUAAGGCCUUAUGUGCCGUAGG
GUAUAGCGGUAUCCCGAACAAUUCCGCUGCUUUGAGUAUUAAGCUGCUAC
AUCAUGUAGCAGC-3′

The inventors have partially deleted and truncated the sequence of the CasRfg.3-sgRNA-scaffold, resulting in a shorter, chemically synthesizable sgRNA that retains activity. The specifically designed truncated scaffold sequences are as follows (SEQ ID NOs: 4-9):

>tracSL2 (SEQ ID NO: 4, 126 nt; deleted
stem-loop 4 and segment 1; shown in
FIG. 11B):
5′-GUUUUAGUUCUAUGUUGGAUAUUGAUAAACUGAUACGAAAUUGUCAG
UUUAUCAAUAUCCAACAAUAGUUCUAAGAUAAGGCCUUAUGUGCCGUAGG
GUAUAGCGGUAUCCCGAACAAUUCCGCUG-3′
>tracSL2-1 (SEQ ID NO: 5, 112 nt; deleted
stem-loop 4 and segment 1, shortened stem-
loop 1; shown in FIG. 11C):
5′-GUUUUAGUUCUAUGUUGGAUAUUGAUAAAGAAAUUUAUCAAUAUCCA
ACAAUAGUUCUAAGAUAAGGCCUUAUGUGCCGUAGGGUAUAGCGGUAUCC
CGAACAAUUCCGCUG-3′
>tracSL2-2 (SEQ ID NO: 6, 114 nt; deleted
stem-loop 4 and segment 1, shortened stem-
loop 1; shown in FIG. 11D):
5′-GUUUUAGUUCUAUGUUGGAUAUUCUGAUACGAAAUUGUCAGAAUAUC
CAACAAUAGUUCUAAGAUAAGGCCUUAUGUGCCGUAGGGUAUAGCGGUAU
CCCGAACAAUUCCGCUG-3′
>tracSL2-1 + 2 (SEQ ID NO: 7, 100 nt;
deleted stem-loop 4 and segment 1,
further shortened stem-loop 1; shown
in FIG. 11E):
5′-GUUUUAGUUCUAUGUUGGAUAUUGAAAAAUAUCCAACAAUAGUUCUA
AGAUAAGGCCUUAUGUGCCGUAGGGUAUAGCGGUAUCCCGAACAAUUCCG
CUG-3′
>trac1-05(SEQ ID NO: 8, 108nt; shown
in FIG. 11 F):
5′-GUUUUAGUUGUUGGAUAUUGAUAAACUGAUACGAAAUUGUCAGUUUA
UCAAUAUCCAACAAGGCCUUAUGUGCCGUAGGGUAUAGCGGUAUCCCGAA
CAAUUCCGCUG-3′
>trac2-05(SEQ ID NO: 9, 105nt;
shown in FIG. 11 G):
5′-GUUUUAGUUGGAUAUUGAUAAACUGAUACGAAAUUGUCAGUUUAUCA
AUAUCCAACAAGGCCUUAUGUGCCGUAGGGUAUAGCGGUAUCCCGAACAA
UUCCGCUG-3′

The secondary structures of the above scaffold sequences were predicted using RNAfold, as shown in FIGS. 11A-G.

The guide sequence (5′-auaacacacagacagcucug-3′ (SEQ ID NO: 24)) was ligated to the 5′ end of each of the above scaffold sequences to generate full-length sgRNA sequences. The full-length sgRNAs were transcribed in vitro and, using a similar method as described in the previous examples, were mixed with Cut Buffer, the CasRfg.3 protein of Embodiment 2, and the CasRfg.3-N2-Target linearized plasmid prepared in Embodiment 4 for cleavage. The cleavage effect was then detected by electrophoresis.

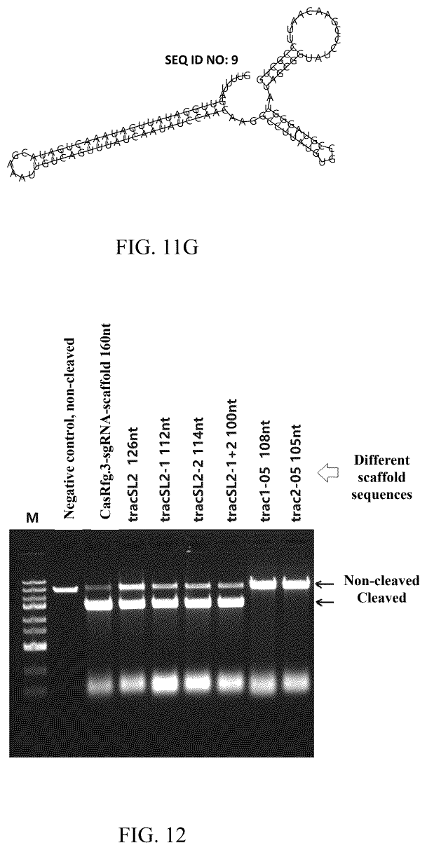

As shown in FIG. 12, the full-length gRNA assembled with the tracSL2, tracSL2-1, tracSL2-2, and tracSL2-1+2 truncated scaffolds retained cleavage activity.

In the CasRfg.3-sgRNA-scaffold, stem-loop 4 and segment 1 are deletable regions; stem-loop 1 may be truncated, for example, its stem portion may be shortened without affecting the complex's cleavage activity.

### Example 8: Testing of Different Specific PAMs

The PAM for the CasRfg.3 protein is 5′-DDRGDNN-3′. Degenerate primers were designed to construct cleavage substrates containing the corresponding spacer sequence (spacer) of CasRfg.3-N2 and different specific PAM sequences, which were used to test cleavage activity under different PAM conditions.

a. PCR Amplification of Cleavage Substrates

Amplification primers are listed in Table 16.

TABLE 16
Amplification primers

| Primer | Sequence |
|---|---|
| Blunt-PR1 | atagggcgaattgaatttagcccaggat |
| | (SEQ ID NO: 28) |
| Ca2PAMs-PF2 | cagagctgtctgtgtgttatttgggag |
| | (SEQ ID NO: 29) |
| Ca2PAMs-PR2 | tcccaaataacacacagacagctctgdd |
| | rgdnngagaatgatggacatttagacac |
| | tgg (SEQ ID NO: 30) |
| Blunt-PF1 | ccctcactaaagggactagtccagggat |
| | (SEQ ID NO: 31) |

The primers listed in the table were diluted to 10 pmol/μl for use. Two sets of primers, Blunt-PR1+Ca2PAMs-PF2 and Ca2PAMs-PR2+Blunt-PF1, were used for PCR amplification targeting the CasRfg.3-N2-Target plasmid, resulting in fragments F1 and F2, respectively. The two fragments were cloned into a pEASY-Blunt-Zero vector (TransGen CB501-01) via Gibson assembly, and plated on Ampicillin plates. Different positive clones were picked and sent for sequencing, obtaining various clones containing the CasRfg.3-N2 corresponding spacer sequence and specific PAM sequences.

b. Activity Testing of Different PAM Substrates

From each of the aforementioned different PAM plasmids, 5 μg was linearized using XmnI, followed by heat-inactivation for 15 minutes a 75° C. to inactivate XmnI. Then, 5 μl of the heat-inactivated product was used as the cleavage substrate. Cleavage experiments were conducted respectively using the following sgRNA:

>tracSL2-1 + 2-full length (where the
lowercase letters indicate the guide
sequence)
(SEQ ID NO: 32)
auaacacacagacagcucugGUUUUAGUUCUAUGUUGGAUAUUGAAAAAU
AUCCAACAAUAGUUCUAAGAUAAGGCCUUAUGUGCCGUAGGGUAUAGCGG
UAUCCCGAACAAUUCCGCUG
>CasRfg.3-sgRNA-full length (where
the lowercase letters indicate the
guide sequence)
(SEQ ID NO: 33)
auaacacacagacagcucugGUUUUAGUUCUAUGUUGGAUAUUGAUAAAC
UGAUACGAAAUUGUCAGUUUAUCAAUAUCCAACAAUAGUUCUAAGAUAAG
GCCUUAUGUGCCGUAGGGUAUAGCGGUAUCCCGAACAAUUCCGCUGCUUU
GAGUAUUAAGCUGCUACAUCAUGUAGCAGC

The in vitro cleavage reaction system for CasRfg.3-N2 with different PAMs is detailed in Table 17.

TABLE 17

| Component | Amount (μL) |
|---|---|
| 10xCut Buffer(200 mM HEPES, 1M NaCl, 50 mM | 3 |
| MgCl₂, 1 mM EDTA) | |
| CasRfg.3-N2 heat-inactivated linearized product with | 5 |
| different PAMs | |
| CasRfg.3-sgRNA-Full Length/tracSL2-1 + 2-Full Length | 5 μg |
| CasRfg.3 protein of Embodiment 2 (10 mg/ml) | 0.3 |
| ddH₂O | Up to 30 |

The reaction was performed at 37° C., and 5 μl of Loading Buffer was added to the cleavage product, from which 15 μl was used for electrophoretic analysis.

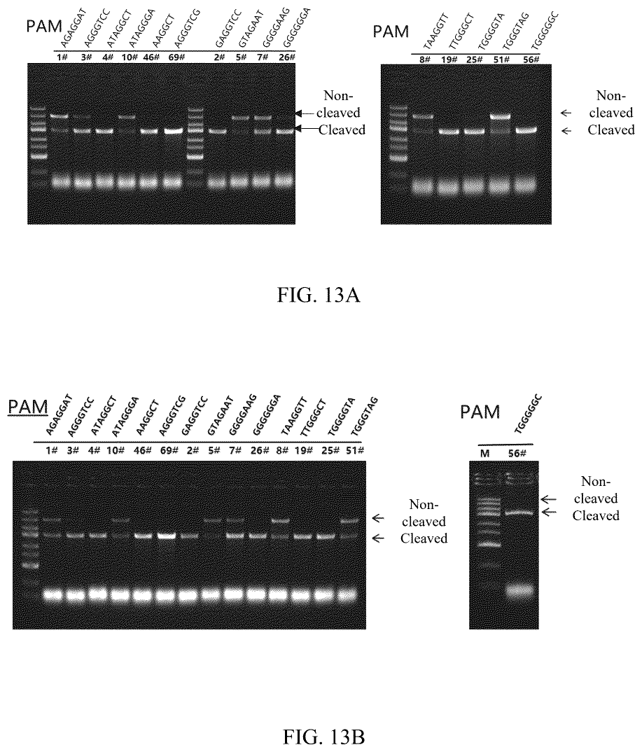

Results are shown in FIGS. 13A and 13B, demonstrating that the CRISPR-CasRfg.3 system can recognize a variety of PAMs, including those shown in Table 18.

TABLE 18

| | | | | |
|---|---|---|---|---|
| 5′-ataggct-3′ | 5′-agggtcg-3′ | 5′-aaggct-3′ | 5′-gaggtcc-3′ | 5′-ttgggct-3′ |
| 5′-tggggta-3′ | 5′-tgggggc-3′ | 5′-gggggga-3′ | 5′-agaggat-3′ | 5′-agggtcc-3′ |
| 5′-ataggga-3′ | 5′-gtagaat-3′ | 5′-ggggaag-3′ | 5′-taaggtt-3′ | 5′-tgggtag-3′ |

### Example 9: Effect of Guide Sequence Length on Cleavage Activity

The inventors utilized truncated gRNA scaffolds to construct sgRNAs with guide sequences of 20 nt-24 nt, and conducted cleavage activity assays.

SgRNAs containing guide sequences of various lengths were obtained by in vitro transcription, with sequences as follows:

>20nt guide sequence (SEQ ID NO: 34):
ataacacacagacagctctgGUUUUAGUUCUAUGUUGGAUAUUGAAAAAU
AUCCAACAAUAGUUCUAAGAUAAGGCCUUAUGUGCCGUAGGGUAUAGCGG
UAUCCCGAACAAUUCCGCUG
>21nt guide sequence (SEQ ID NO: 35):
aataacacacagacagctctgGUUUUAGUUCUAUGUUGGAUAUUGAAAAA
UAUCCAACAAUAGUUCUAAGAUAAGGCCUUAUGUGCCGUAGGGUAUAGCG
GUAUCCCGAACAAUUCCGCUG
>22nt guide sequence (SEQ ID NO: 36):
aaataacacacagacagctctgGUUUUAGUUCUAUGUUGGAUAUUGAAAA
AUAUCCAACAAUAGUUCUAAGAUAAGGCCUUAUGUGCCGUAGGGUAUAGC
GGUAUCCCGAACAAUUCCGCUG
>23nt guide sequence (SEQ ID NO: 37):
caaataacacacagacagctctgGUUUUAGUUCUAUGUUGGAUAUUGAAA
AAUAUCCAACAAUAGUUCUAAGAUAAGGCCUUAUGUGCCGUAGGGUAUAG
CGGUAUCCCGAACAAUUCCGCUG
>24nt guide sequence (SEQ ID NO: 38):
ccaaataacacacagacagctctgGUUUUAGUUCUAUGUUGGAUAUUGAA
AAAUAUCCAACAAUAGUUCUAAGAUAAGGCCUUAUGUGCCGUAGGGUAUA
GCGGUAUCCCGAACAAUUCCGCUG

The aforementioned sgRNAs were respectively mixed with Cut Buffer, the CasRfg.3 protein expressed in Example 2, and the CasRfg.3-N2-Target linearized plasmid prepared in Example 4. The reaction was conducted at 37° C. for 30 minutes. 15 μl of the cleavage product was taken, mixed with 3 μl Loading Buffer, and subsequently subjected to electrophoresis to assess cleavage effects.

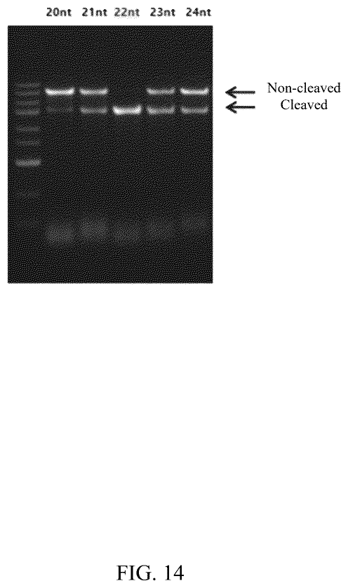

As shown in FIG. 14, CasRfg.3 exhibited cleavage activity across all guide lengths from 20 nt to 24 nt, with the highest activity observed at 22 nt.

### Example 10: No DNA Cleavage Activity Detected for Specific Similar Protein Publicly Available from NCBI

NCBI discloses a similar protein (Reference
Sequence: WP_085022725.1), annotated as Cas9
nuclease, which the inventors have named
Ca9-89, with the amino acid sequence (SEQ ID
NO: 39):
MKRKVGFGLDVGIGSVGFAVLSYDKVYDARIEQVGVRLFDSGEEVKNHKR
ASKNQGRRQYRSGRRLIRRRYHRKERAKRFIERIGLLSAAKIKEWQEVNG
NQNIYSIRFRGLSEKLTPEEIADCVIHFCNHRGYREFYEDDVDEKEAGKI
KTALFRFDEKMTEGKYVSVADMILHDKEFATDTQFPNFHNHKNDDEEKYF
LIKRAALRDELRAILQKQQEYYKQLTDQNIAFLCDEIVFVQRDFEDGPGD
KNDKNRKFMGFLDTIGCCMFYKEELRGFRSTVIADIYSLVNGLSQMMYVD
STTGEITFLPEAADDIIEFALKNASITEKDIKKILEKYNLTLIKAEKLEE
NIPQTIKTLKVLKKVLDASGYSYDELIQEEQFDFDKPSKLHELCNLLASN
ITPKRRRKALEKAGWNKDFQAQTKRIHFGGTSNVCYRYMLEAIDAFRHGE
LYGNFQARRKQEQLTDEAENTERVKLLPPFTKEMDEDVVKNVVVFKAINE
TRKIINALIGKYGSPAYINIEVADELGHSIETRRKMTKANNDNMKKKEAI
GAKLVELGLRKEGEVSGKDIARYRLWEQQNGIDLYTGNNIPEADVLSGQY
DVDHIIPFSLILDDTLNNKVLTGMGSNRQAKSNTAPREYLSDKAEAEFIK
RVNVLLKKKISKKKYQYLMVKNLRDSKLLNEWKSRNINDTRYISRFLANY
LNNTLIFNSDKKKNVYAINGAITSRMRKMWLNKKTWGNPEKNRENNLHHA
ADAIVIANLTPAAVELASDNLKLONIFRQNGKRVTEEYENYLDRAVRKIE
KYYHFNPELAKKLLVSKDRIPSMVRLLREETDKRLVDPSLEEFKMVTPES
FRQNLEQYYNDPEFVASIQMPLVSYKQSKRFSGSFTKDKPIKKKEREDSS
TVKIDSLGNENILDAKSYYCLEVYSTKDNKTALRGLRYVDFKLKDKKMFI
TVPNPENYGKHIMYLFKNDYIVVYNKKGEEKARGFYSSVKAITRDQLYLK
DNNTNVDIIFTIKKDDTVKKYHIDILGQIGGEIKCSAPFLSITEKE.
NCBI also discloses the genome sequence of
the bacterium from which the protein is
derived, and the inventors accordingly
analyzed its DR sequence (SEQ ID NO: 40):
5′-GTTTTAGTTCTATGTTGGATATTGATAAACTGATA-3′;
and
the tracrRNA sequence (SEQ ID NO: 41):
5′-TTGTCAGTTTATCAATATCCAACAATAGTTCTAAGATAAGGCCTTAT
GTGCCGTAGGGTATAGCGGTATCCCGAACAATTCCGCTCCTTTGTTCATT
AAGCTGCTACATTATGTAGCGGCTTTT-3′.

A plasmid vector pXC09-89-GFPgRNA (SEQ ID NO: 42) was prepared by conventional methods. The vector contains the coding sequence for the Ca9-89 recombinant protein and the corresponding sgRNA coding sequence (the DR sequence and tracrRNA sequence were joined into the sgRNA scaffold using the commonly used GAAA linker in the field). The plasmid map is shown in FIG. 16.

The pXC09-89-GFPgRNA plasmid was transfected into 293T cells using Lipofectamine 2000. After 48 hours of transfection, cell lysis was performed using Western and IP cell lysis buffer (Biyuntian P0013). The samples were centrifuged for 5 minutes, and the supernatant was collected and filtered through a 0.45 μm membrane. The protein was then purified using a heparin column (Cytiva HiTrap™ Heparin HP), followed by ion exchange chromatography (Cytiva HiTrap™ Q FF) and size exclusion chromatography (Cytiva Superdex™ 200 Increase 10/300 GL). The purified Ca9-89 recombinant protein was analyzed by SDS-PAGE, which revealed a single band.

The sgRNA corresponding to Ca9-89 was obtained by in vitro transcription.

>Ca9-89sgRNA sequence (SEQ ID NO: 43):
GAACGGCUCGGAGAUCAUCAUUGCGGUUUUAGUUCUAUGUUGGAUAUUGA
UAAACUGAUAGAAAUUGUCAGUUUAUCAAUAUCCAACAAUAGUUCUAAGA
UAAGGCCUUAUGUGCCGUAGGGUAUAGCGGUAUCCCGAACAAUUCCGCUC
CUUUGUUCAUUAAGCUGCUACAUUAUGUAGCGGC.

Using the method of Example 3, the Ca9-89 recombinant protein was subjected to an in vitro cleavage reaction with sgRNA, 10× Cut Buffer, and the in vitro cleavage substrate from Example 3. The reaction was carried out at 37° C. for 3 hours and then at 75° C. for 15 minutes. Following the same procedure as in Example 3, the presumed cleaved fragments were end-repaired by adding biotin-labeled Adapter1 to the nicked ends through a ligation reaction. The presumed cleaved products with biotin-labeled adapters were then specifically bound to streptavidin-labeled magnetic beads (Beads) for purification. The specifically purified fragments were amplified by PCR, but the concentration measured was only 0.355 ng/μl, which did not meet the requirements for NGS sequencing. This result also indicates that the Ca9-89 protein has no detectable DNA cleavage activity or very weak activity. Therefore, the CasRfg.3 protein of the present disclosure achieved an unexpected technical effect.

### Example 11: Detection of Single Base Editing Activity of Ca2 Fusion Protein

### A. Cloning and Construction of Ca2 Protein Single-Base Editing System

The Escherichia coli BL21 strain contains the β-galactosidase gene. The genomic sequence of the BL21-Gold (DE3) pLysS AG strain, with GenBank accession number CP001665.1, was obtained via NCBI search. Sequence analysis was conducted to locate the β-galactosidase gene ECBD_3313 sequence, and a sgRNA targeting this gene matching the Ca2 protein PAM sequence was designed. Additionally, competent cells of the BL21 (DE3) pLysS strain (Shanghai Weidi Bioscience, CAT #: EC1003) were used to confirm the ECBD_3313 sequence through PCR amplification and sequencing verification. Sequences for the PCR validation primers, the finalized ECBD_3313 sequence, and the designed sgRNA sequence are as follows:

PCR validation primer sequences:
Primer LacZKO-PF1: (SEQ ID NO: 44)
ACGCCATCAAAAATAATTCGCGTC.
Primer LacZKO-PR1: (SEQ ID NO: 45)
CCGACTGGAAAGCGGGCAGTG
Sequence obtained from PCR amplification
and sequencing of the ECBD_3313:
>ECBD_3313 (SEQ ID NO: 46):
atgaccatgattacggattcactggccgtcgttttacaacgtcgtgactg
ggaaaaccctggcgttacccaacttaatcgccttgcagcacatccccctt
tcgccagctggcgtaatagcgaagaggcccgcaccgatcgcccttcccaa
cagttgcgcagcctgaatggcgaatggcgctttgcctggtttccggcacc
agaagcggtgccggaaagctggctggagtgcgatcttcctgaggccgata
ctgtcgtcgtcccctcaaactggcagatgcacggttacgatgcgcccatc
tacaccaacgtgacctatcccattacggtcaatccgccgtttgttcccac
ggagaatccgacgggttgttactcgctcacatttaatgttgatgaaagct
ggctacaggaaggccagacgcgaattatttttgatggcgt
sgRNA sequence (SEQ ID NO: 47):
5′-uauuacgccagcuggcgaaaGUUUUAGUUCUAUGUUGGAUAUUGAUA
AACUGAUACGAAAUUGUCAGUUUAUCAAUAUCCAACAAUAGUUCUAAGAU
AAGGCCUUAUGUGCCGUAGGGUAUAGCGGUAUCCCGAACAAUUCCGCUGC
UUUGAGUAUUAAGCUGCUACAUCAUGUAGCAGC-3′ (here, uauuac
gccagcuggegaaa (SEQ ID NO: 48) is the guide
sequence, and the immediate neighboring PAM
sequence of the guide sequence in ECBD_3313
is 5′-gggggat-3′).

The deaminase (APOBEC-1 Domain) and uracil DNA glycosylase inhibitor (UGI Domain) were fused to the N-terminal and C-terminal of the Ca2 protein, respectively, resulting in the APOBEC-1-Ca2-UGI fusion protein (SEQ ID NO: 49).

Using molecular biology methods, a single-base editing vector targeting the Escherichia coli β-galactosidase gene was constructed using the aforementioned APOBEC-1-Ca2-UGI fusion protein to obtain the plasmid CBE-Ca2-LacZgRNA02 (SEQ ID NO: 50). The fusion protein expression was driven by the lac UV5 promoter, while the sgRNA expression was driven by the J23119 promoter.

### B. Single-Base Editing Detection

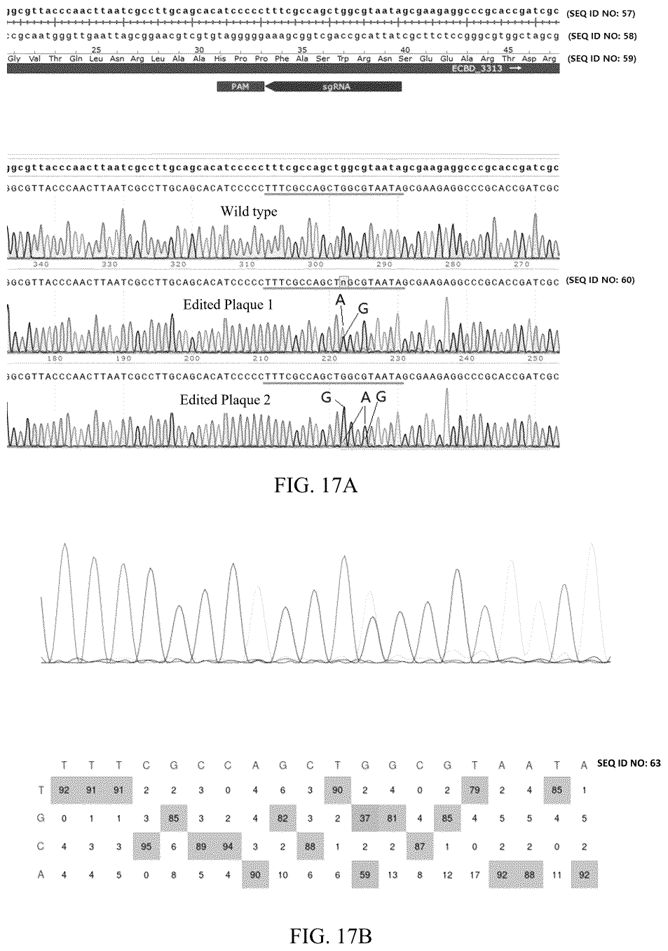

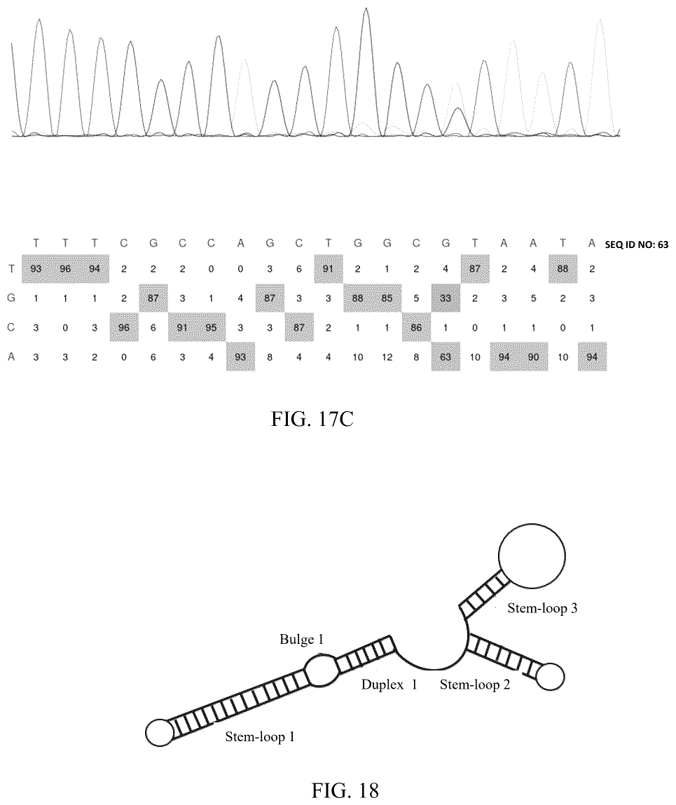

The CBE-Ca2-LacZgRNA02 plasmid was transformed via heat shock into BL21 (DE3) pLysS competent cells (Shanghai Weidi Bioscience, CAT #: EC1003). After heat shock transformation, LB medium without antibiotics was added to incubate for 2 hours. The mixture was then plated on Ampicillin plates and incubated overnight. Single colonies (Plaque 1 and Plaque 2) were picked and inoculated into LB medium containing 100 μg/ml ampicillin for 4 hours. PCR amplification was conducted on the cultured bacterial solution using primers LacZKO-PF1 and LacZKO-PR1 along with a high-fidelity PCR enzyme (Ezinkin Biosciences UltraHiPF™ DNA Polymerase Kit, CAT #: PC019). The PCR product was sent for Sanger sequencing (FIG. 17A) to analyze the conversion of C to T at the sgRNA target site, thus evaluating the single-base editing activity of the Ca2 fusion protein.

The aforementioned sequencing results indicate that the Ca2 fusion protein can convert a C to a T in the sgRNA target site, leading to overlapping peaks of A+G in the sequencing chromatogram, which demonstrates the single-base editing activity of the Ca2 fusion protein. In Plaques 1 and 2, the G→A mutation efficiency is relatively high, reaching 59% and 63% respectively, as shown in FIGS. 17B and 17C, where FIG. 17B corresponds to Plaque 1 and FIG. 17C corresponds to Plaque 2.

The above-described embodiments express only several embodiments of the present disclosure, which are described in a more specific and detailed manner, but are not to be construed as a limitation on the scope of the present disclosure. It should be pointed out that, for a person of ordinary skill in the art, deformations and improvements may be made without departing from the conception of the present disclosure, which all fall within the scope of protection of the present disclosure. Therefore, the scope of protection of the present disclosure shall be subject to the appended claims. Also, all of the documents referred to in the present disclosure are cited as references in the present disclosure as if each document had been individually cited as a reference.

While the above describes specific embodiments of the present disclosure, it should be understood by those skilled in the art that these are merely illustrative, and that a variety of changes or modifications may be made to these embodiments without departing from the principles and substance of the present disclosure. Thus, the scope of protection of the present disclosure is limited by the appended claims.

1. An isolated Cas9 protein, comprising a sequence having at least 90% identity to the sequence set forth in SEQ ID NO: 1, wherein
the Cas9 protein is capable of forming a complex with a guide RNA, binding to a target nucleic acid under the guidance of the guide RNA, or cleaving or modifying the target nucleic acid under the guidance of the guide RNA.

2. The Cas9 protein of claim 1, wherein the guide RNA includes a guide sequence, a direct repeat sequence, and a tracrRNA sequence, wherein the guide sequence is complementary to the target nucleic acid, and the tracrRNA is capable of interacting with the Cas9 protein and the direct repeat sequence.

3. The Cas9 protein of claim 1, wherein the guide RNA includes no more than three stem-loop structures.

4. The Cas9 protein of claim 1, wherein the Cas9 protein is capable of performing one or more of the following functions at a temperature of 25° C. to 55° C.:
forming a complex with the guide RNA,
binding to the target nucleic acid under the guidance of the guide RNA, and
cleaving or modifying the target nucleic acid under the guidance of the guide RNA.

5. The Cas9 protein of claim 1, wherein a PAM sequence recognized by the Cas9 protein is 5′-DDRGDNN-3′, wherein D represents A, G, or T; R represents A or G; and N represents A, C, G, or T.

6. The Cas9 protein of claim 1, wherein the Cas9 protein is a conservative variant polypeptide or a homolog of the polypeptide set forth in SEQ ID NO: 1.

7. A fusion protein, comprising: the Cas9 protein of claim 1 and a heterologous functional domain fused thereto.

8. The fusion protein of claim 7, wherein the heterologous functional domain is fused to an N-terminus, a C-terminus of the Cas9 protein, or within the Cas9 protein.

9. The fusion protein of claim 7, wherein the heterologous functional domain is optionally selected from one or more of: a reporter protein or detection label, a localization signal, a deaminase, a DNA binding domain, an epitope tag, a transcription activation domain, a transcription repression domain, a nuclease, a methyltransferase, a demethylase, a histone deacetylase, a DNA or RNA ligase.

10. The fusion protein of claim 7, further comprising a linker between the heterologous functional domain and the Cas9 protein.

11. A conjugate, comprising: the Cas9 protein of claim 1 and a heterologous functional moiety conjugated thereto.

12. A guide RNA directing the Cas9 protein of claim 1 to bind to a target sequence, the guide RNA comprising a guide sequence, a direct repeat sequence, and a tracrRNA sequence, wherein
the guide sequence is complementary to the target sequence, and the tracrRNA is capable of interacting with the Cas9 protein and the direct repeat sequence; and
the guide RNA has a sequence set forth in any one of SEQ ID NOs: 3-7.

13. An isolated polynucleotide encoding the Cas9 protein of claim 1.

14. A recombinant vector comprising:
the polynucleotide of claim 13.

15. The recombinant vector of claim 14, wherein the polynucleotide is further operatively linked to at least one of a promoter, an enhancer, and a terminator.

16. A recombinant cell comprising the recombinant vector of claim 14.

17. A delivery system comprising:
a delivery vector; and
the Cas9 protein of claim 1.

18. A method for binding, cleaving, or modifying a target nucleic acid, comprising: binding, cleaving, or modifying the target nucleic acid with a complex of the Cas9 protein of claim 1.

19. (canceled)

20. A method for detecting the presence of a target nucleic acid in a test sample, comprising: binding a complex of the Cas9 protein of claim 1 with a guide RNA to the target nucleic acid, wherein
a fusion protein or a conjugate contains the Cas9 protein and a detectable label; and when the complex binds to the target nucleic acid, the fusion protein or the conjugate containing the Cas9 protein binds, cleaves, or modifies the target nucleic acid, thereby analyzing the presence of the target nucleic acid in the test sample by observing the presence of the detectable label.

21. A composition comprising: the Cas9 protein of claim 1.


SEQUENCE LISTING (63 sequences)

SEQ ID NO: 1 - protein, 1046 aa, Anaerovibrio sp.
MKRKIGIGLDVGIGSVGFAVLSYDKVYDARIEQVGVRLFDSGEEPKTKVSKNQGRRQYRAGRRLIRRRYHRKERAKRFIERIGLLSADKIKEWQEVNGNQNIYYVRFRGLSEKLTPQEIADCVIHFCNHRGYREFYEDDVADEKEAGKIKTALSRFDEKMAAGKYVSVADMILHDNEFSTNTQFPDFHNHKGDDDEKYFLIKRAALRDELRAILRKQQEFYEQLTEHNIDFLCDDIVFVQRDFEDGPGDKTDKKRKFMGFLDSIGCCMFYKEEVRGYRSTVIADIYSLVNGLSKMMYVDSTTGEITFLPEAADKIIDFALKNASITEKDIKEILKKYNLTLIKAEKLEENIPQTIKTLKILKKILDDSGYSYDELIKEEQFDFDRPSKLHELCSLLASNITPKRRRKALEKAGWNKALQEQTRRIHFGGTSNVCYRYMLEAIDAFRHGELYGNFQARRNKEQLTDEAENTEKVRLLPPFTKEMDEDVVKNVVVFKAINETRKIINALIGKYGSPAYINIEVADELGHSIETRRKMTKANNEKMKKKEAISTKLVELGLRKEGEVSGKDIARYRLWEQQDGIDLYTGENIPEADVLSGQYDIDHIIPFSLILDDTLNNKVLTGMGSNRQAKSNKAPREYLSDKAELEFIKRVNILLKKKIISKKKYKYLMVKNLRDSKLLDEWKSRNINDTRYISRFLVNYLNNMLLFNSDKKKNVYAINGAITSKMRKLWLNKRTWGTPEKNRENNLHHAADAIVIANLTPAAVELASDNLKLQNIFRQNGKRVTEEYDNYLDKAVRKMEKYYHLNEELAKNLLVRKDRIPSMVRSLREETDKRLVDTSLEEFNMVTPEAFRKNLEQYYNDSDFVASIQMPLVSYKQSKRFSGSFTKDNPIKKKDKEDSSSVKMDSLGNENILDAKSYYCLEVYSTKDNKTALRGLRYVDFKLKNKKMFINVPNPDNYDKHIMYLFKNDYIVVYNKKGEERIKGYYTGIKNIKANRFYLISNNDAVRKDFTLSKDDTIKKYHIDILGQIGGEVKCSAPFLSITEKE

SEQ ID NO: 2 - DNA, 3141 bp, Anaerovibrio sp.
  source 1..3141: mol_type = genomic DNA
atgaaaagaaaaattggaatcgggttggatgttggtataggttctgtaggttttgctgtattgtcgtatgataaggtatatgatgcccgtatcgaacaggtaggggtccggctttttgattctggggaagaacccaaaacaaaagtcagtaagaaccaggggagacggcaatatagggctggtcgtagattgattcggcgtagataccatcgtaaagagcgggcaaagagatttattgagcggataggtttattaagtgcggacaaaattaaagaatggcaggaagtaaatggcaatcagaatatttactatgtgagatttaggggattgtcggaaaagctcaccccacaagaaattgccgattgcgttatccatttttgcaaccatagaggttatcgtgaattctatgaagatgatgttgcggatgaaaaggaagctggcaagataaagaccgccctatccagatttgatgaaaaaatggcagctggtaagtatgtatccgttgccgatatgattttgcatgataatgaattttctacaaacacccagtttccagatttccataatcataaaggtgatgacgacgaaaaatattttcttattaaacgggcagcgttaagggatgaacttagggcaatactccggaagcaacaggaattctatgaacagttgacggaacataatattgatttcttatgtgacgacatagtttttgttcagcgggattttgaggatggacccggggataaaactgataaaaagcgtaagtttatgggttttttggatagcataggctgctgtatgttttataaggaagaagttagaggctataggtccacagtaattgcagatatttattccttggtaaacggtttgtccaagatgatgtatgttgacagcaccactggtgaaataacatttttaccggaagctgctgacaaaattattgattttgccctaaaaaatgcatccattactgaaaaagatatcaaagaaatactgaagaaatacaatttgacccttataaaggctgaaaagctagaggaaaacataccgcagactataaagactctgaagatattgaaaaagatattggatgatagtgggtatagttatgatgaattgatcaaggaagaacaatttgattttgataggccatccaaactccatgaattatgtagtttgttagccagcaatattaccccaaaacgcagaagaaaagcattggaaaaagctggctggaataaagcattgcaagaacaaaccaggagaatacactttggtggaacatcaaatgtatgctatcgctacatgctagaagctattgatgccttcagacatggtgaattatatggtaatttccaagcccgacgaaataaggaacagcttacagatgaagcagaaaatacggagaaggtaagattattaccaccgttcacaaaagaaatggatgaagatgtggtaaagaatgtagtggttttcaaggcaatcaatgaaacccgcaagataatcaatgcccttataggcaagtacggttcgccggcatatatcaatattgaggtggctgatgaactggggcacagtattgaaaccaggcggaaaatgaccaaagccaacaacgaaaaaatgaaaaaaaaggaggctattagtacaaagctggttgaactgggacttcgtaaagagggggaagtatctggtaaagatatagcgcgttatcgtctatgggagcagcaggatgggatagatttatatacgggggaaaatattccagaagcggatgtgctgagtgggcaatatgatattgatcacattatcccattttcgctgatattggatgataccctaaataataaggtactgactggtatggggagcaatcgtcaggcaaaatccaataaagctcctagagaatatttatctgataaggctgaattggagtttataaagagagtaaatatcttactgaagaaaaaaataatctcaaagaaaaagtacaagtacttgatggtaaagaaccttcgtgacagtaaactgctggatgagtggaagtcaagaaatattaacgacaccagatatataagtcgttttttagtgaactatctaaataatatgttgctatttaacagcgataaaaagaaaaatgtatatgcaatcaatggtgccattacatccaaaatgcgaaaactgtggcttaataagcgaacctggggaactccggaaaagaacagggaaaataatttgcatcatgcagctgatgctatagttattgccaatcttactccagctgcagtggaactggccagtgataaccttaaactccagaatattttccgtcaaaatggcaagcgagtgacggaggaatatgataattatcttgataaggcagttcgcaagatggagaagtattatcacttaaatgaagaattggccaagaatctgctggtacgaaaggatcgtattccatccatggtaaggtcattgcgggaggaaacggataagagattggtggatacaagtcttgaggaattcaatatggttacaccagaagcattccgcaaaaatctagagcagtattataatgactctgattttgttgcttccatacagatgccgttggtatcctataagcagtccaagcggttttctggaagctttactaaggataatcctataaagaaaaaagataaagaagactcatcaagtgtaaaaatggactctttaggcaatgaaaatattttggatgccaaatcgtattactgtttggaggtgtattccacaaaagataataagacggctttgcgcggccttaggtatgttgattttaaattgaaaaacaaaaagatgtttatcaatgttcctaatccagataattatgataagcatattatgtatttgtttaagaatgattatattgtggtgtacaacaaaaaaggagaggaaagaattaaaggttattatacaggaataaaaaacatcaaagctaatagattttatttgataagtaacaatgatgcggtgcgaaaagattttacattatctaaagatgatactataaaaaaatatcacattgatattcttgggcaaattggtggtgaggtaaaatgttccgctccattcttgtcaataacggagaaagaataa

SEQ ID NO: 3 - RNA, 160 nt, synthetic construct
  source 1..160: mol_type = other RNA
gttttagttctatgttggatattgataaactgatacgaaattgtcagtttatcaatatccaacaatagttctaagataaggccttatgtgccgtagggtatagcggtatcccgaacaattccgctgctttgagtattaagctgctacatcatgtagcagc

SEQ ID NO: 4 - RNA, 126 nt, synthetic construct
  source 1..126: mol_type = other RNA
gttttagttctatgttggatattgataaactgatacgaaattgtcagtttatcaatatccaacaatagttctaagataaggccttatgtgccgtagggtatagcggtatcccgaacaattccgctg

SEQ ID NO: 5 - RNA, 112 nt, synthetic construct
  source 1..112: mol_type = other RNA
gttttagttctatgttggatattgataaagaaatttatcaatatccaacaatagttctaagataaggccttatgtgccgtagggtatagcggtatcccgaacaattccgctg

SEQ ID NO: 6 - RNA, 114 nt, synthetic construct
  source 1..114: mol_type = other RNA
gttttagttctatgttggatattctgatacgaaattgtcagaatatccaacaatagttctaagataaggccttatgtgccgtagggtatagcggtatcccgaacaattccgctg

SEQ ID NO: 7 - RNA, 100 nt, synthetic construct
  source 1..100: mol_type = other RNA
gttttagttctatgttggatattgaaaaatatccaacaatagttctaagataaggccttatgtgccgtagggtatagcggtatcccgaacaattccgctg

SEQ ID NO: 8 - RNA, 108 nt, synthetic construct
  source 1..108: mol_type = other RNA
gttttagttgttggatattgataaactgatacgaaattgtcagtttatcaatatccaacaaggccttatgtgccgtagggtatagcggtatcccgaacaattccgctg

SEQ ID NO: 9 - RNA, 105 nt, synthetic construct
  source 1..105: mol_type = other RNA
gttttagttggatattgataaactgatacgaaattgtcagtttatcaatatccaacaaggccttatgtgccgtagggtatagcggtatcccgaacaattccgctg

SEQ ID NO: 10 - DNA, 3138 bp, synthetic construct
  source 1..3138: mol_type = other DNA
atgaagagaaagatcggcatcggcctggacgtgggcatcggcagcgtgggcttcgccgtgctgagctacgacaaggtgtacgacgccagaatcgagcaggtgggcgtgagactgttcgacagcggcgaggagcccaagaccaaggtgagcaagaaccagggcagaagacagtacagagccggcagaagactgatcagaagaagataccacagaaaggagagagccaagagattcatcgagagaatcggcctgctgagcgccgacaagatcaaggagtggcaggaggtgaacggcaaccagaacatctactacgtgagattcagaggcctgagcgagaagctgaccccccaggagatcgccgactgcgtgatccacttctgcaaccacagaggctacagagagttctacgaggacgacgtggccgacgagaaggaggccggcaagatcaagaccgccctgagcagattcgacgagaagatggccgccggcaagtacgtgagcgtggccgacatgatcctgcacgacaacgagttcagcaccaacacccagttccccgacttccacaaccacaagggcgacgacgacgagaagtacttcctgatcaagagagccgccctgagagacgagctgagagccatcctgagaaagcagcaggagttctacgagcagctgaccgagcacaacatcgacttcctgtgcgacgacatcgtgttcgtgcagagagacttcgaggacggccccggcgacaagaccgacaagaagagaaagttcatgggcttcctggacagcatcggctgctgcatgttctacaaggaggaggtgagaggctacagaagcaccgtgatcgccgacatctacagcctggtgaacggcctgagcaagatgatgtacgtggacagcaccaccggcgagatcaccttcctgcccgaggccgccgacaagatcatcgacttcgccctgaagaacgccagcatcaccgagaaggacatcaaggagatcctgaagaagtacaacctgaccctgatcaaggccgagaagctggaggagaacatcccccagaccatcaagaccctgaagatcctgaagaagatcctggacgacagcggctacagctacgacgagctgatcaaggaggagcagttcgacttcgacagacccagcaagctgcacgagctgtgcagcctgctggccagcaacatcacccccaagagaagaagaaaggccctggagaaggccggctggaacaaggccctgcaggagcagaccagaagaatccacttcggcggcaccagcaacgtgtgctacagatacatgctggaggccatcgacgccttcagacacggcgagctgtacggcaacttccaggccagaagaaacaaggagcagctgaccgacgaggccgagaacaccgagaaggtgagactgctgccccccttcaccaaggagatggacgaggacgtggtgaagaacgtggtggtgttcaaggccatcaacgagacaagaaagatcatcaacgccctgatcggcaagtacggcagccccgcctacatcaacatcgaggtggccgacgagctgggccacagcatcgagacaagaagaaagatgaccaaggccaacaacgagaagatgaagaagaaggaggccatcagcaccaagctggtggagctgggcctgagaaaggagggcgaggtgagcggcaaggacatcgccagatacagactgtgggagcagcaggacggcatcgacctgtacaccggcgagaacatccccgaggccgacgtgctgagcggccagtacgacatcgaccacatcatccccttcagcctgatcctggacgacaccctgaacaacaaggtgctgaccggcatgggcagcaacagacaggccaagagcaacaaggcccccagagagtacctgagcgacaaggccgagctggagttcatcaagagagtgaacatcctgctgaagaagaagatcatcagcaagaagaagtacaagtacctgatggtgaagaacctgagagacagcaagctgctggacgagtggaagagcagaaacatcaacgacaccagatacatcagcagattcctggtgaactacctgaacaacatgctgctgttcaacagcgacaagaagaagaacgtgtacgccatcaacggcgccatcaccagcaagatgagaaagctgtggctgaacaagagaacctggggcacccccgagaagaacagagagaacaacctgcaccacgccgccgacgccatcgtgatcgccaacctgacccccgccgccgtggagctggccagcgacaacctgaagctgcagaacatcttcagacagaacggcaagagagtgaccgaggagtacgacaactacctggacaaggccgtgagaaagatggagaagtactaccacctgaacgaggagctggccaagaacctgctggtgagaaaggacagaatccccagcatggtgagaagcctgagagaggagacagacaagagactggtggacaccagcctggaggagttcaacatggtgacccccgaggccttcagaaagaacctggagcagtactacaacgacagcgacttcgtggccagcatccagatgcccctggtgagctacaagcagagcaagagattcagcggcagcttcaccaaggacaaccccatcaagaagaaggacaaggaggacagcagcagcgtgaagatggacagcctgggcaacgagaacatcctggacgccaagagctactactgcctggaggtgtacagcaccaaggacaacaagaccgccctgagaggcctgagatacgtggacttcaagctgaagaacaagaagatgttcatcaacgtgcccaaccccgacaactacgacaagcacatcatgtacctgttcaagaacgactacatcgtggtgtacaacaagaagggcgaggagagaatcaagggctactacaccggcatcaagaacatcaaggccaacagattctacctgatcagcaacaacgacgccgtgagaaaggacttcaccctgagcaaggacgacaccatcaagaagtaccacatcgacatcctgggccagatcggcggcgaggtgaagtgcagcgcccccttcctgagcatcaccgagaaggag

SEQ ID NO: 11 - RNA, 36 nt, synthetic construct
  source 1..36: mol_type = other RNA
gttttagttctatgttggatattgataaactgatac

SEQ ID NO: 12 - RNA, 120 nt, synthetic construct
  source 1..120: mol_type = other RNA
ttgtcagtttatcaatatccaacaatagttctaagataaggccttatgtgccgtagggtatagcggtatcccgaacaattccgctgctttgagtattaagctgctacatcatgtagcagc

SEQ ID NO: 13 - DNA, 8641 bp, synthetic construct
  source 1..8641: mol_type = other DNA; plasmid = CasRfg.3-pET28a
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccccggcagctaagaaaaagaaactggatggcagcgtcgacatgaagagaaagatcggcatcggcctggacgtgggcatcggcagcgtgggcttcgccgtgctgagctacgacaaggtgtacgacgccagaatcgagcaggtgggcgtgagactgttcgacagcggcgaggagcccaagaccaaggtgagcaagaaccagggcagaagacagtacagagccggcagaagactgatcagaagaagataccacagaaaggagagagccaagagattcatcgagagaatcggcctgctgagcgccgacaagatcaaggagtggcaggaggtgaacggcaaccagaacatctactacgtgagattcagaggcctgagcgagaagctgaccccccaggagatcgccgactgcgtgatccacttctgcaaccacagaggctacagagagttctacgaggacgacgtggccgacgagaaggaggccggcaagatcaagaccgccctgagcagattcgacgagaagatggccgccggcaagtacgtgagcgtggccgacatgatcctgcacgacaacgagttcagcaccaacacccagttccccgacttccacaaccacaagggcgacgacgacgagaagtacttcctgatcaagagagccgccctgagagacgagctgagagccatcctgagaaagcagcaggagttctacgagcagctgaccgagcacaacatcgacttcctgtgcgacgacatcgtgttcgtgcagagagacttcgaggacggccccggcgacaagaccgacaagaagagaaagttcatgggcttcctggacagcatcggctgctgcatgttctacaaggaggaggtgagaggctacagaagcaccgtgatcgccgacatctacagcctggtgaacggcctgagcaagatgatgtacgtggacagcaccaccggcgagatcaccttcctgcccgaggccgccgacaagatcatcgacttcgccctgaagaacgccagcatcaccgagaaggacatcaaggagatcctgaagaagtacaacctgaccctgatcaaggccgagaagctggaggagaacatcccccagaccatcaagaccctgaagatcctgaagaagatcctggacgacagcggctacagctacgacgagctgatcaaggaggagcagttcgacttcgacagacccagcaagctgcacgagctgtgcagcctgctggccagcaacatcacccccaagagaagaagaaaggccctggagaaggccggctggaacaaggccctgcaggagcagaccagaagaatccacttcggcggcaccagcaacgtgtgctacagatacatgctggaggccatcgacgccttcagacacggcgagctgtacggcaacttccaggccagaagaaacaaggagcagctgaccgacgaggccgagaacaccgagaaggtgagactgctgccccccttcaccaaggagatggacgaggacgtggtgaagaacgtggtggtgttcaaggccatcaacgagacaagaaagatcatcaacgccctgatcggcaagtacggcagccccgcctacatcaacatcgaggtggccgacgagctgggccacagcatcgagacaagaagaaagatgaccaaggccaacaacgagaagatgaagaagaaggaggccatcagcaccaagctggtggagctgggcctgagaaaggagggcgaggtgagcggcaaggacatcgccagatacagactgtgggagcagcaggacggcatcgacctgtacaccggcgagaacatccccgaggccgacgtgctgagcggccagtacgacatcgaccacatcatccccttcagcctgatcctggacgacaccctgaacaacaaggtgctgaccggcatgggcagcaacagacaggccaagagcaacaaggcccccagagagtacctgagcgacaaggccgagctggagttcatcaagagagtgaacatcctgctgaagaagaagatcatcagcaagaagaagtacaagtacctgatggtgaagaacctgagagacagcaagctgctggacgagtggaagagcagaaacatcaacgacaccagatacatcagcagattcctggtgaactacctgaacaacatgctgctgttcaacagcgacaagaagaagaacgtgtacgccatcaacggcgccatcaccagcaagatgagaaagctgtggctgaacaagagaacctggggcacccccgagaagaacagagagaacaacctgcaccacgccgccgacgccatcgtgatcgccaacctgacccccgccgccgtggagctggccagcgacaacctgaagctgcagaacatcttcagacagaacggcaagagagtgaccgaggagtacgacaactacctggacaaggccgtgagaaagatggagaagtactaccacctgaacgaggagctggccaagaacctgctggtgagaaaggacagaatccccagcatggtgagaagcctgagagaggagacagacaagagactggtggacaccagcctggaggagttcaacatggtgacccccgaggccttcagaaagaacctggagcagtactacaacgacagcgacttcgtggccagcatccagatgcccctggtgagctacaagcagagcaagagattcagcggcagcttcaccaaggacaaccccatcaagaagaaggacaaggaggacagcagcagcgtgaagatggacagcctgggcaacgagaacatcctggacgccaagagctactactgcctggaggtgtacagcaccaaggacaacaagaccgccctgagaggcctgagatacgtggacttcaagctgaagaacaagaagatgttcatcaacgtgcccaaccccgacaactacgacaagcacatcatgtacctgttcaagaacgactacatcgtggtgtacaacaagaagggcgaggagagaatcaagggctactacaccggcatcaagaacatcaaggccaacagattctacctgatcagcaacaacgacgccgtgagaaaggacttcaccctgagcaaggacgacaccatcaagaagtaccacatcgacatcctgggccagatcggcggcgaggtgaagtgcagcgcccccttcctgagcatcaccgagaaggagacaggcggcggccccggcggcggcgccgccgccggcagcggcagccctaagaaaaaacgaaaagttggcagcggaagcaaaaggccggcggccacgaaaaaggccggccaggcaaaaaagaaaaagtaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 14 - DNA, 273 bp, synthetic construct
  source 1..273: mol_type = other DNA
ggagttcagacgtgtgctcttccgatctcagcacaaaaggaaactcaccctaactgtaaagtaattgtgtgttttgagactataaatatgcatgcgagaaaagccttgtttgccaccatggaacggctcggagatcatcattgcgnnnnnnngtgagcaagggcgaggagctgttcaccggggtggtgcccatcctggtcgagctggacggcgacgtaaacggccacaagttcagcgtgtccggcagatcggaagagcacacgtctgaactcc

SEQ ID NO: 15 - RNA, 185 nt, synthetic construct
  source 1..185: mol_type = other RNA
gaacggctcggagatcatcattgcggttttagttctatgttggatattgataaactgatacgaaattgtcagtttatcaatatccaacaatagttctaagataaggccttatgtgccgtagggtatagcggtatcccgaacaattccgctgctttgagtattaagctgctacatcatgtagcagc

SEQ ID NO: 16 - DNA, 59 bp, synthetic construct
  source 1..59: PCR_primers = fwd_seq:gttgacatgctggattgagacttcctacactctttc cctacacgacgctcttccgatct,rev_seq:gatcggaagagcgtcgtgtagggaaa gagtgtaggaagtctcaatccagcatgtcaac; mol_type = other DNA
gttgacatgctggattgagacttcctacactctttccctacacgacgctcttccgatct

SEQ ID NO: 17 - DNA, 58 bp, synthetic construct
  source 1..58: PCR_primers = fwd_seq:gttgacatgctggattgagacttcctacactctttc cctacacgacgctcttccgatct,rev_seq:gatcggaagagcgtcgtgtagggaaa gagtgtaggaagtctcaatccagcatgtcaac; mol_type = other DNA
gatcggaagagcgtcgtgtagggaaagagtgtaggaagtctcaatccagcatgtcaac

SEQ ID NO: 18 - DNA, 19 bp, synthetic construct
  source 1..19: PCR_primers = fwd_seq:ggagttcagacgtgtgctc,rev_seq:gttgacat gctggattgagacttc; mol_type = other DNA
ggagttcagacgtgtgctc

SEQ ID NO: 19 - DNA, 24 bp, synthetic construct
  source 1..24: PCR_primers = fwd_seq:ggagttcagacgtgtgctc,rev_seq:gttgacat gctggattgagacttc; mol_type = other DNA
gttgacatgctggattgagacttc

SEQ ID NO: 20 - DNA, 58 bp, synthetic construct
  source 1..58: PCR_primers = fwd_seq:aatgatacggcgaccaccgagatctacactatagcc tacactctttccctacacgacg,rev_seq:caagcagaagacggcatacgagatcga gtaatgtgactggagttcagacgtgtgctc; mol_type = other DNA
aatgatacggcgaccaccgagatctacactatagcctacactctttccctacacgacg

SEQ ID NO: 21 - DNA, 57 bp, synthetic construct
  source 1..57: PCR_primers = fwd_seq:aatgatacggcgaccaccgagatctacactatagcc tacactctttccctacacgacg,rev_seq:caagcagaagacggcatacgagatcga gtaatgtgactggagttcagacgtgtgctc; mol_type = other DNA
caagcagaagacggcatacgagatcgagtaatgtgactggagttcagacgtgtgctc

SEQ ID NO: 22 - DNA, 532 bp, synthetic construct
  source 1..532: mol_type = other DNA
gaatgggctatttctaagcacggcaggtaccgtcttccccttcccccgccaacacagctggcccaggacaagaaggtgcagagacaaacagggggcaccctgcctgtcctgctaggaactgcaaggcagcgtgccgcacacgctgcctgggccaatgccacccaggccaggagagggtttggggccagacaccagcccatacccaagggtcccaggggatgtggggagaaggggaatccaccttttccttccctcccacctcccaaataacacacagacagctctgttggtctgagaatgatggacatttagacactggcgccaggtttgcgcctgaccggcgccacgcaggggtgggcggagcaaagacacacaggtgggctacaggtgtcacacggcaccagccagggcccggggtggctggggtgaggatgggtgtttggccagtgaccaggagtcaggtcaagtccaggtggtcagtgccaggggctccaggaggggagggcagtgccataaccctcctggtgtccag

SEQ ID NO: 23 - DNA, 500 bp, synthetic construct
  source 1..500: mol_type = other DNA
ggcactgtgccctccagattcctcagagcctggtgccaggtgcacctccccagccctctcagcccctgagctctgccaccctgcccgggcttctgcccttgggtcctggcctctcagggtctcctcagctctgcaaggaggggctgggagcaggtcccaggctagcagtgggctggaggcctcctgtgtggtctctacctgtccatgccgaggggtcacctctgggtgaggtcgcacccacccccctcacggggctgctttgccagaaggtctgttcccctcatggcctctgctactctttacacttagttcttcccaggaagggcccggtggctgcagataatgtttcccatctgtggagccttcaagggctgggcccatgaggattctgctcccagcttgctcttcctctccagcctgcacccaccaggccccagaatcctgaggatggagctgctcctgtcccctgaggtggtctaaacctctggggaggcactgga

SEQ ID NO: 24 - DNA, 3194 bp, synthetic construct
  source 1..3194: mol_type = other DNA; plasmid = CasRfg.3-N2-Target
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtccagggatctggacaccaggagggttatggcactgccctcccctcctggagcccctggcactgaccacctggacttgacctgactcctggtcactggccaaacacccatcctcaccccagccaccccgggccctggctggtgccgtgtgacacctgtagcccacctgtgtgtctttgctccgcccacccctgcgtggcgccggtcaggcgcaaacctggcgccagtgtctaaatgtccatcattctcagaccaacagagctgtctgtgtgttatttgggaggtgggagggaaggaaaaggtggattccccttctccccacatcccctgggacccttgggtatgggctggtgtctggccccaaaccctctcctggcctgggtggcattggcccaggcagcgtgtgcggcacgctgccttgcagttcctagcaggacaggcagggtgccccctgtttgtctctgcaccttcttgtcctgggccagctgtgttggcgggggaaggggaagacggtacctgccgtgcttagaaatagcccattcatcctgggctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaggcgcgccacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 25 - DNA, 3162 bp, synthetic construct
  source 1..3162: mol_type = other DNA; plasmid = CasRfg.3-N4-Target
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtccagggatggcactgtgccctccagattcctcagagcctggtgccaggtgcacctccccagccctctcagcccctgagctctgccaccctgcccgggcttctgcccttgggtcctggcctctcagggtctcctcagctctgcaaggaggggctgggagcaggtcccaggctagcagtgggctggaggcctcctgtgtggtctctacctgtccatgccgaggggtcacctctgggtgaggtcgcacccacccccctcacggggctgctttgccagaaggtctgttcccctcatggcctctgctactctttacacttagttcttcccaggaagggcccggtggctgcagataatgtttcccatctgtggagccttcaagggctgggcccatgaggattctgctcccagcttgctcttcctctccagcctgcacccaccaggccccagaatcctgaggatggagctgctcctgtcccctgaggtggtctaaacctctggggaggcactggaatcctgggctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaggcgcgccacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 26 - RNA, 180 nt, synthetic construct
  source 1..180: mol_type = other RNA
ataacacacagacagctctggttttagttctatgttggatattgataaactgatacgaaattgtcagtttatcaatatccaacaatagttctaagataaggccttatgtgccgtagggtatagcggtatcccgaacaattccgctgctttgagtattaagctgctacatcatgtagcagc

SEQ ID NO: 27 - RNA, 180 nt, synthetic construct
  source 1..180: mol_type = other RNA
ccgaggggtcacctctgggtgttttagttctatgttggatattgataaactgatacgaaattgtcagtttatcaatatccaacaatagttctaagataaggccttatgtgccgtagggtatagcggtatcccgaacaattccgctgctttgagtattaagctgctacatcatgtagcagc

SEQ ID NO: 28 - DNA, 28 bp, synthetic construct
  source 1..28: PCR_primers = fwd_seq:ccctcactaaagggactagtccagggat,rev_seq :atagggcgaattgaatttagcccaggat; mol_type = other DNA
atagggcgaattgaatttagcccaggat

SEQ ID NO: 29 - DNA, 27 bp, synthetic construct
  source 1..27: PCR_primers = fwd_seq:cagagctgtctgtgtgttatttgggag,rev_seq: tcccaaataacacacagacagctctgddrgdnngagaatgatggacatttagacactg g; mol_type = other DNA
cagagctgtctgtgtgttatttgggag

SEQ ID NO: 30 - DNA, 59 bp, synthetic construct
  source 1..59: PCR_primers = fwd_seq:cagagctgtctgtgtgttatttgggag,rev_seq: tcccaaataacacacagacagctctgddrgdnngagaatgatggacatttagacactg g; mol_type = other DNA
tcccaaataacacacagacagctctgddrgdnngagaatgatggacatttagacactgg

SEQ ID NO: 31 - DNA, 28 bp, synthetic construct
  source 1..28: PCR_primers = fwd_seq:ccctcactaaagggactagtccagggat,rev_seq :atagggcgaattgaatttagcccaggat; mol_type = other DNA
ccctcactaaagggactagtccagggat

SEQ ID NO: 32 - RNA, 120 nt, synthetic construct
  source 1..120: mol_type = other RNA
ataacacacagacagctctggttttagttctatgttggatattgaaaaatatccaacaatagttctaagataaggccttatgtgccgtagggtatagcggtatcccgaacaattccgctg

SEQ ID NO: 33 - RNA, 180 nt, synthetic construct; identical to SEQ ID NO: 26
  source 1..180: mol_type = other RNA

SEQ ID NO: 34 - RNA, 120 nt, synthetic construct; identical to SEQ ID NO: 32
  source 1..120: mol_type = other RNA

SEQ ID NO: 35 - RNA, 121 nt, synthetic construct
  source 1..121: mol_type = other RNA
aataacacacagacagctctggttttagttctatgttggatattgaaaaatatccaacaatagttctaagataaggccttatgtgccgtagggtatagcggtatcccgaacaattccgctg

SEQ ID NO: 36 - RNA, 122 nt, synthetic construct
  source 1..122: mol_type = other RNA
aaataacacacagacagctctggttttagttctatgttggatattgaaaaatatccaacaatagttctaagataaggccttatgtgccgtagggtatagcggtatcccgaacaattccgctg

SEQ ID NO: 37 - RNA, 123 nt, synthetic construct
  source 1..123: mol_type = other RNA
caaataacacacagacagctctggttttagttctatgttggatattgaaaaatatccaacaatagttctaagataaggccttatgtgccgtagggtatagcggtatcccgaacaattccgctg

SEQ ID NO: 38 - RNA, 124 nt, synthetic construct
  source 1..124: mol_type = other RNA
ccaaataacacacagacagctctggttttagttctatgttggatattgaaaaatatccaacaatagttctaagataaggccttatgtgccgtagggtatagcggtatcccgaacaattccgctg

SEQ ID NO: 39 - protein, 1046 aa, Anaerovibrio sp.
MKRKVGFGLDVGIGSVGFAVLSYDKVYDARIEQVGVRLFDSGEEVKNHKRASKNQGRRQYRSGRRLIRRRYHRKERAKRFIERIGLLSAAKIKEWQEVNGNQNIYSIRFRGLSEKLTPEEIADCVIHFCNHRGYREFYEDDVDEKEAGKIKTALFRFDEKMTEGKYVSVADMILHDKEFATDTQFPNFHNHKNDDEEKYFLIKRAALRDELRAILQKQQEYYKQLTDQNIAFLCDEIVFVQRDFEDGPGDKNDKNRKFMGFLDTIGCCMFYKEELRGFRSTVIADIYSLVNGLSQMMYVDSTTGEITFLPEAADDIIEFALKNASITEKDIKKILEKYNLTLIKAEKLEENIPQTIKTLKVLKKVLDASGYSYDELIQEEQFDFDKPSKLHELCNLLASNITPKRRRKALEKAGWNKDFQAQTKRIHFGGTSNVCYRYMLEAIDAFRHGELYGNFQARRKQEQLTDEAENTERVKLLPPFTKEMDEDVVKNVVVFKAINETRKIINALIGKYGSPAYINIEVADELGHSIETRRKMTKANNDNMKKKEAIGAKLVELGLRKEGEVSGKDIARYRLWEQQNGIDLYTGNNIPEADVLSGQYDVDHIIPFSLILDDTLNNKVLTGMGSNRQAKSNTAPREYLSDKAEAEFIKRVNVLLKKKISKKKYQYLMVKNLRDSKLLNEWKSRNINDTRYISRFLANYLNNTLIFNSDKKKNVYAINGAITSRMRKMWLNKKTWGNPEKNRENNLHHAADAIVIANLTPAAVELASDNLKLQNIFRQNGKRVTEEYENYLDRAVRKIEKYYHFNPELAKKLLVSKDRIPSMVRLLREETDKRLVDPSLEEFKMVTPESFRQNLEQYYNDPEFVASIQMPLVSYKQSKRFSGSFTKDKPIKKKEREDSSTVKIDSLGNENILDAKSYYCLEVYSTKDNKTALRGLRYVDFKLKDKKMFITVPNPENYGKHIMYLFKNDYIVVYNKKGEEKARGFYSSVKAITRDQLYLKDNNTNVDIIFTIKKDDTVKKYHIDILGQIGGEIKCSAPFLSITEKE

SEQ ID NO: 40 - DNA, 35 bp, synthetic construct
  source 1..35: mol_type = other DNA
gttttagttctatgttggatattgataaactgata

SEQ ID NO: 41 - DNA, 124 bp, synthetic construct
  source 1..124: mol_type = other DNA
ttgtcagtttatcaatatccaacaatagttctaagataaggccttatgtgccgtagggtatagcggtatcccgaacaattccgctcctttgttcattaagctgctacattatgtagcggctttt

SEQ ID NO: 42 - DNA, 7381 bp, synthetic construct
  source 1..7381: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgaagagaaaggtgggcttcggcctggacgtgggcatcggcagcgtgggcttcgccgtgctgagctacgacaaggtgtacgacgctagaatcgagcaagtgggcgtgagactgttcgacagcggcgaagaggtgaagaaccacaagagagctagcaagaaccaaggccggagacagtacagaagcggccggagactgatcagacggagataccacagaaaggagagagccaagagattcatcgagagaatcggcctgctgagcgccgccaagatcaaggagtggcaagaggtgaacggcaatcagaacatctacagcatcagattcagaggcctgagcgagaagctgacccccgaggagatcgccgactgcgtgatccacttctgcaaccacagaggctacagagagttctacgaggacgacgtggacgagaaggaggccggcaagatcaagaccgccctgttcagattcgacgagaagatgaccgagggcaagtacgtgagcgtggccgacatgatcctgcacgacaaggagttcgccaccgacacacagttccccaacttccacaaccacaagaacgacgacgaggagaagtacttcctgatcaagagagccgccctgagagacgaactgagagccatcctgcagaagcagcaagagtactacaagcagctgaccgatcagaacatcgccttcctgtgcgacgagatcgtgttcgtgcagagagacttcgaggacggccccggcgacaagaacgacaagaacagaaagttcatgggcttcctggacaccatcggctgctgcatgttctacaaggaggagctgagaggcttcagaagcaccgtgatcgccgacatctacagcctggtgaacggcctgagccaaatgatgtacgtggacagcaccaccggcgagatcaccttcctgcccgaggccgccgacgacatcatcgagttcgccctgaagaacgctagcatcacagaaaaggacatcaagaagatcctggagaagtacaacctgaccctgatcaaggccgagaagctggaggagaacatccctcagaccatcaagaccctgaaggtgctgaagaaggtgctggacgctagcggctacagctacgacgagctgatccaagaggagcagttcgacttcgacaagcctagcaagctgcacgagctgtgcaacctgctggctagcaacatcacccctaagagacggagaaaggccctggagaaggccggctggaacaaggacttccaagctcagaccaagagaatccacttcggcggcacaagcaacgtgtgctacagatacatgctggaggccatcgacgccttcagacacggcgagctgtacggcaacttccaagctagaagaaagcaagagcagctgaccgacgaggccgagaacaccgagagagtgaagctgctgccccccttcacaaaggagatggacgaggacgtggtgaagaacgtggtcgtgttcaaggccatcaacgagacaagaaagatcatcaacgccctgatcggcaagtacggcagccccgcctacatcaacatcgaggtggccgacgagctgggccacagcatcgagacaagaagaaagatgaccaaggccaacaacgacaacatgaagaaaaaggaggctatcggcgccaagctggtggagctgggcctgagaaaggagggcgaggtgagcggcaaggacatcgctagatacagactgtgggagcagcagaacggcatcgacctgtacaccggcaacaacatccccgaggccgacgtgctgagcgggcagtacgacgtggaccacatcatccccttcagcctgatcctggacgacaccctgaacaacaaggtgctgaccggcatgggcagcaacagacaagccaagagcaacaccgcccctagagagtacctgagcgacaaggccgaggccgagttcatcaagagagtgaacgtgctgctgaagaaaaagatcagcaagaaaaagtatcagtacctgatggtgaagaacctgagagacagcaagctgctgaacgagtggaagagcagaaacatcaacgacacaagatacatcagcagattcctggccaactacctgaacaacaccctgatcttcaacagcgacaagaaaaagaacgtgtacgccatcaacggcgccatcacaagcagaatgagaaagatgtggctgaacaaaaagacctggggcaaccccgagaagaacagagagaacaacctgcaccacgccgccgatgccatcgtgatcgccaacctgacccccgccgccgtggagctggctagcgacaacctgaagctgcagaacatcttccggcagaacgggaaaagagtgaccgaggagtacgagaactacctggacagagccgtgagaaagatcgagaagtactaccacttcaaccccgagctggccaagaagctgctggtgagcaaggacagaatccctagcatggtgagactgctgagagaggaaaccgacaagagactggtggaccctagcctggaggagttcaagatggtgacccccgagagcttcagacagaacctggagcagtactacaacgaccccgagttcgtggctagcattcagatgcccctggtgagctacaagcagagcaagagattcagcggcagcttcaccaaggacaagcccatcaagaaaaaggagagagaggacagcagcaccgtgaagatcgacagcctgggcaacgagaacatcctggacgccaagagctactactgcctggaggtgtacagcacaaaagacaacaaaaccgctctgcggggcctgagatacgtggacttcaagctgaaggacaagaagatgttcatcaccgtgcccaaccccgagaattatggcaagcacatcatgtacctgttcaagaacgactacatcgtggtgtacaacaagaagggcgaggagaaggctagaggcttctacagcagcgtgaaggccatcacaagagatcagctgtacctgaaggacaacaacaccaacgtggacatcatcttcaccatcaagaaggacgacaccgtgaagaagtaccacattgacatcctggggcagatcggcggcgagatcaagtgcagcgcccccttcctgagcatcaccgagaaggagacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccaaaaaaagccgctacataatgtagcagcttaatgaacaaaggagcggaattgttcgggataccgctataccctacggcacataaggccttatcttagaactattgttggatattgataaactgacaatttctatcagtttatcaatatccaacatagaactaaaaccgcaatgatgatctccgagccgttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - RNA, 184 nt, synthetic construct
  source 1..184: mol_type = other RNA
gaacggctcggagatcatcattgcggttttagttctatgttggatattgataaactgatagaaattgtcagtttatcaatatccaacaatagttctaagataaggccttatgtgccgtagggtatagcggtatcccgaacaattccgctcctttgttcattaagctgctacattatgtagcggc

SEQ ID NO: 44 - DNA, 19 bp, synthetic construct
  source 1..19: PCR_primers = fwd_seq:acgccacaaaaaaacgcgc,rev_seq:ccgactgg aaagcgggcagtg; mol_type = other DNA
acgccacaaaaaaacgcgc

SEQ ID NO: 45 - DNA, 21 bp, synthetic construct
  source 1..21: PCR_primers = fwd_seq:acgccacaaaaaaacgcgc,rev_seq:ccgactgg aaagcgggcagtg; mol_type = other DNA
ccgactggaaagcgggcagtg

SEQ ID NO: 46 - DNA, 440 bp, synthetic construct
  source 1..440: mol_type = other DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgt

SEQ ID NO: 47 - RNA, 180 nt, synthetic construct
  source 1..180: mol_type = other RNA
tattacgccagctggcgaaagttttagttctatgttggatattgataaactgatacgaaattgtcagtttatcaatatccaacaatagttctaagataaggccttatgtgccgtagggtatagcggtatcccgaacaattccgctgctttgagtattaagctgctacatcatgtagcagc

SEQ ID NO: 48 - RNA, 20 nt, synthetic construct
  source 1..20: mol_type = other RNA
tattacgccagctggcgaaa

SEQ ID NO: 49 - protein, 1569 aa, synthetic construct
  PEPTIDE 1..1569: note = APOBEC-1-Ca2-UGI fusion protein
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSVDKRKIGIGLDVGIGSVGFAVLSYDKVYDARIEQVGVRLFDSGEEPKTKVSKNQGRRQYRAGRRLIRRRYHRKERAKRFIERIGLLSADKIKEWQEVNGNQNIYYVRFRGLSEKLTPQEIADCVIHFCNHRGYREFYEDDVADEKEAGKIKTALSRFDEKMAAGKYVSVADMILHDNEFSTNTQFPDFHNHKGDDDEKYFLIKRAALRDELRAILRKQQEFYEQLTEHNIDFLCDDIVFVQRDFEDGPGDKTDKKRKFMGFLDSIGCCMFYKEEVRGYRSTVIADIYSLVNGLSKMMYVDSTTGEITFLPEAADKIIDFALKNASITEKDIKEILKKYNLTLIKAEKLEENIPQTIKTLKILKKILDDSGYSYDELIKEEQFDFDRPSKLHELCSLLASNITPKRRRKALEKAGWNKALQEQTRRIHFGGTSNVCYRYMLEAIDAFRHGELYGNFQARRNKEQLTDEAENTEKVRLLPPFTKEMDEDVVKNVVVFKAINETRKIINALIGKYGSPAYINIEVADELGHSIETRRKMTKANNEKMKKKEAISTKLVELGLRKEGEVSGKDIARYRLWEQQDGIDLYTGENIPEADVLSGQYDIDHIIPFSLILDDTLNNKVLTGMGSNRQAKSNKAPREYLSDKAELEFIKRVNILLKKKIISKKKYKYLMVKNLRDSKLLDEWKSRNINDTRYISRFLVNYLNNMLLFNSDKKKNVYAINGAITSKMRKLWLNKRTWGTPEKNRENNLHHAADAIVIANLTPAAVELASDNLKLQNIFRQNGKRVTEEYDNYLDKAVRKMEKYYHLNEELAKNLLVRKDRIPSMVRSLREETDKRLVDTSLEEFNMVTPEAFRKNLEQYYNDSDFVASIQMPLVSYKQSKRFSGSFTKDNPIKKKDKEDSSSVKMDSLGNENILDAKSYYCLEVYSTKDNKTALRGLRYVDFKLKNKKMFINVPNPDNYDKHIMYLFKNDYIVVYNKKGEERIKGYYTGIKNIKANRFYLISNNDAVRKDFTLSKDDTIKKYHIDILGQIGGEVKCSAPFLSITEKESGGSGGSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFPKKKRKVGSGSKRPAATKKAGQAKKKKLEYPYDVPDYA

SEQ ID NO: 50 - DNA, 7730 bp, synthetic construct
  source 1..7730: mol_type = other DNA; plasmid = CBE-Ca2-LacZgRNA02
catgattccttcatatttgcatatacgagcggccgccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcaattgacattgattattgactagtccgggtttacactttatgcttccggctcgtataatgtgtggattttgagttaggatctaaagaggagaaaaccggtgccaccatgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgtcgacaagagaaagatcggcatcggcctggacgtgggcatcggcagcgtgggcttcgccgtgctgagctacgacaaggtgtacgacgccagaatcgagcaggtgggcgtgagactgttcgacagcggcgaggagcccaagaccaaggtgagcaagaaccagggcagaagacagtacagagccggcagaagactgatcagaagaagataccacagaaaggagagagccaaaagattcatcgagagaatcggcctgctgagcgccgacaagatcaaggagtggcaggaggtgaacggcaaccagaacatctactacgtgagattcagaggcctgagcgagaagctgaccccccaggagatcgccgactgcgtgatccacttctgcaaccacagaggctacagagagttctacgaggacgacgtggccgacgagaaggaggccggcaagatcaagaccgccctgagcagattcgacgagaagatggccgccggcaagtacgtgagcgtggccgacatgatcctgcacgacaacgagttcagcaccaacacccagttccccgacttccacaaccacaagggcgacgacgacgagaagtacttcctgatcaagagagccgccctgagagacgagctgagagccatcctgagaaagcagcaggagttctacgagcagctgaccgagcacaacatcgacttcctgtgcgacgacatcgtgttcgtgcagagagacttcgaggacggccccggcgacaagaccgacaagaagagaaagttcatgggcttcctggacagcatcggctgctgcatgttctacaaggaggaggtgagaggctacagaagcaccgtgatcgccgacatctacagcctggtgaacggcctgagcaagatgatgtacgtggacagcaccaccggcgagatcaccttcctgcccgaggccgccgacaagatcatcgacttcgccctgaagaacgccagcatcaccgagaaggacatcaaggagatcctgaagaagtacaacctgaccctgatcaaggccgagaagctggaggagaacatcccccagaccatcaagaccctgaagatcctgaagaagatcctggacgacagcggctacagctacgacgagctgatcaaggaggagcagttcgacttcgacagacccagcaagctgcacgagctgtgcagcctgctggccagcaacatcacccccaagagaagaagaaaggccctggagaaggccggctggaacaaggccctgcaggagcagaccagaagaatccacttcggcggcaccagcaacgtgtgctacagatacatgctggaggccatcgacgccttcagacacggcgagctgtacggcaacttccaggccagaagaaacaaggagcagctgaccgacgaggccgagaacaccgagaaggtgagactgctgccccccttcaccaaggagatggacgaggacgtggtgaagaacgtggtggtgttcaaggccatcaacgagacaagaaagatcatcaacgccctgatcggcaagtacggcagccccgcctacatcaacatcgaggtggccgacgagctgggccacagcatcgagacaagaagaaagatgaccaaggccaacaacgagaagatgaagaagaaggaggccatcagcaccaagctggtggagctgggcctgagaaaggagggcgaggtgagcggcaaggacatcgccagatacagactgtgggagcagcaggacggcatcgacctgtacaccggcgagaacatccccgaggccgacgtgctgagcggccagtacgacatcgaccacatcatccccttcagcctgatcctggacgacaccctgaacaacaaggtgctgaccggcatgggcagcaacagacaggccaagagcaacaaggcccccagagagtacctgagcgacaaggccgagctggagttcatcaagagagtgaacatcctgctgaagaagaagatcatcagcaagaagaagtacaagtacctgatggtgaagaacctgagagacagcaagctgctggacgagtggaagagcagaaacatcaacgacaccagatacatcagcagattcctggtgaactacctgaacaacatgctgctgttcaacagcgacaagaagaagaacgtgtacgccatcaacggcgccatcaccagcaagatgagaaagctgtggctgaacaagagaacctggggcacccccgagaagaacagagagaacaacctgcaccacgccgccgacgccatcgtgatcgccaacctgacccccgccgccgtggagctggccagcgacaacctgaagctgcagaacatcttcagacagaacggcaagagagtgaccgaggagtacgacaactacctggacaaggccgtgagaaagatggagaagtactaccacctgaacgaggagctggccaagaacctgctggtgagaaaggacagaatccccagcatggtgagaagcctgagagaggagacagacaagagactggtggacaccagcctggaggagttcaacatggtgacccccgaggccttcagaaagaacctggagcagtactacaacgacagcgacttcgtggccagcatccagatgcccctggtgagctacaagcagagcaagagattcagcggcagcttcaccaaggacaaccccatcaagaagaaggacaaggaggacagcagcagcgtgaagatggacagcctgggcaacgagaacatcctggacgccaagagctactactgcctggaggtgtacagcaccaaggacaacaagaccgccctgagaggcctgagatacgtggacttcaagctgaagaacaagaagatgttcatcaacgtgcccaaccccgacaactacgacaagcacatcatgtacctgttcaagaacgactacatcgtggtgtacaacaagaagggcgaggagagaatcaagggctactacaccggcatcaagaacatcaaggccaacagattctacctgatcagcaacaacgacgccgtgagaaaggacttcaccctgagcaaggacgacaccatcaagaagtaccacatcgacatcctgggccagatcggcggcgaggtgaagtgcagcgcccccttcctgagcatcaccgagaaggagagcggcgggagcggcgggagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaatttcctaagaaaaaacgaaaagttggcagcggaagcaaaaggccggcggccacgaaaaaggccggccaggcaaaaaagaaaaagctcgagtacccatacgatgttccagattacgcttgagaattctagaataacctgaggtaccgttaacaaataaaacgaaaggctcagtcgaaagactgggcctttcgttttatctgttgtttgcggtgaacgctctcgtcgacttgacagctagctcagtcctaggtataatactagttattacgccagctggcgaaagttttagttctatgttggatattgataaactgatacgaaattgtcagtttatcaatatccaacaatagttctaagataaggccttatgtgccgtagggtatagcggtatcccgaacaattccgctgctttgagtattaagctgctacatcatgtagcagcttttttctcgaggtttaaacagatct

SEQ ID NO: 51 - DNA, 94 bp, synthetic construct
  source 1..94: mol_type = other DNA
cgcaaacctggcgccagtgtctaaatgtccatcattctcagaccaacagagctgtctgtgtgttatttgggaggtgggagggaaggaaaaggtg

SEQ ID NO: 52 - DNA, 49 bp, synthetic construct
  source 1..49: mol_type = other DNA
cgcaaacctggcgccagtgtctaaatgtccatcattctcagaccaacag

SEQ ID NO: 53 - DNA, 45 bp, synthetic construct
  source 1..45: mol_type = other DNA
agctgtctgtgtgttatttgggaggtgggagggaaggaaaaggtg

SEQ ID NO: 54 - DNA, 103 bp, synthetic construct
  source 1..103: mol_type = other DNA
tggaggcctcctgtgtggtctctacctgtccatgccgaggggtcacctctgggtgaggtcgcacccacccccctcacggggctgctttgccagaaggtctgtt

SEQ ID NO: 55 - DNA, 51 bp, synthetic construct
  source 1..51: mol_type = other DNA
tggaggcctcctgtgtggtctctacctgtccatgccgaggggtcacctctg

SEQ ID NO: 56 - DNA, 52 bp, synthetic construct
  source 1..52: mol_type = other DNA
ggtgaggtcgcacccacccccctcacggggctgctttgccagaaggtctgtt

SEQ ID NO: 57 - DNA, 80 bp, synthetic construct
  source 1..80: mol_type = other DNA
gcgttacccaacttaatcgccttgcagcacatccccctttcgccagctggcgtaatagcgaagaggcccgcaccgatcgc

SEQ ID NO: 58 - DNA, 80 bp, synthetic construct
  source 1..80: mol_type = other DNA
cgcaatgggttgaattagcggaacgtcgtgtagggggaaagcggtcgaccgcattatcgcttctccgggcgtggctagcg

SEQ ID NO: 59 - protein, 27 aa, synthetic construct
GVTQLNRLAAHPPFASWRNSEEARTDR

SEQ ID NO: 60 - DNA, 80 bp, synthetic construct
  source 1..80: mol_type = other DNA
  variation 49: note = n is a or g
gcgttacccaacttaatcgccttgcagcacatccccctttcgccagctngcgtaatagcgaagaggcccgcaccgatcgc

SEQ ID NO: 61 - protein, 1368 aa, Anaerovibrio sp. [sequence omitted]

SEQ ID NO: 62 - protein, 1053 aa, Anaerovibrio sp.
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKAYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 63 - DNA, 20 bp, synthetic construct
  source 1..20: mol_type = other DNA
tttcgccagctggcgtaata